(19) United States
(12) Patent Application Publication
(10) Pub. No.: US 2024/0417809 A1
(43) Pub. Date: Dec. 19, 2024

(54) METHOD FOR DIAGNOSING HEMATOLOGICAL MALIGNANCIES AND ASSOCIATED KIT

(71) Applicants: UNIVERSITE DE ROUEN, Mont-Saint-Aignan (FR); CENTRE HENRI BECQUEREL, Rouen (FR); INSTITUT NATIONAL DE LA SANTE ET DE LA RECHERCHE MEDICALE, Paris Cedex 13 (FR)

(72) Inventors: Philippe RUMINY, Rouen (FR); Vinciane MARCHAND, Rouen (FR); Fabrice JARDIN, Rouen (FR)

(21) Appl. No.: 18/757,985

(22) Filed: Jun. 28, 2024

Related U.S. Application Data
(63) Continuation of application No. 14/917,087, filed on Mar. 7, 2016.

(30) Foreign Application Priority Data
Sep. 11, 2013 (FR) 13 58721

(51) Int. Cl. C12Q 1/6886

(52) U.S. Cl. CPC C12Q 1/6886; C12Q 2600/156; C12Q 2600/158

(57) ABSTRACT

A method for diagnosing a cancer in a subject, notably with the aim of finding fusion transcripts, includes an RT-MLPA step carried out on a biological sample obtained from the subject using the probes SEQ ID NO: 1 to 25, 30, 31 and 113 to 120, and/or with at least the probes SEQ ID NO: 374 to 405, and/or with at least the probes SEQ ID NO: 524 to 559, each of the probes being fused, at one end at least, with a priming sequence.

### CROSS REFERENCE TO RELATED APPLICATIONS

This application is a Continuation of application Ser. No. 14/917,087, filed on Mar. 7, 2016, which is the National Phase under 35 U.S.C. § 371 of International Application No. PCT/FR2014/052255, filed on Sep. 11, 2014, which claims the benefit under 35 U.S.C. § 119 (a) to Patent Application No. 13 58721, filed in France on Sep. 11, 2013, all of which are hereby expressly incorporated by reference into the present application.

### REFERENCE TO AN ELECTRONIC SEQUENCE LISTING

The contents of the electronic sequence listing (7179-0450_SEQ_LISTING.xml; Size: 1,015,242 bytes; and Date of Creation: Jun. 26, 2024) is herein incorporated by reference in its entirety.

### BACKGROUND OF THE INVENTION

### (1) Field of the Invention

The present invention relates to a method for diagnosing a cancer in a subject, comprising a step of RT-MLPA on a biological sample obtained from said subject using at least one pair of probes selected from:
probes SEQ ID NOs: 1 to 25, 30, 31 and 113 to 120,
probes SEQ ID NOs: 374 to 405, and
probes SEQ ID NOs: 524 to 559,
   each of the probes being fused, at one end at least, with a priming sequence.

Preferably, the present invention relates to a method for diagnosing a cancer in a subject, comprising an RT-MLPA step carried out on a biological sample obtained from said subject, notably with the aim of finding fusion transcripts, using the probes SEQ ID NOs: 1 to 25, 30, 31 and 113 to 120, and/or using the probes SEQ ID NOs: 374 to 405, and/or using at least the probes SEQ ID NOs: 524 to 559, each of the probes being fused, at one end at least, with a priming sequence. The present invention also relates to a kit comprising at least the probes SEQ ID NOs: 1 to 25, 30, 31 and 113 to 120, and/or at least the probes SEQ ID NOs: 374 to 405, and/or at least the probes SEQ ID NOs: 524 to 559, each of the probes being fused, at one end at least, with a priming sequence.

### (2) Description of the Related Art

Cancers are due to accumulation of genetic abnormalities by tumor cells. These abnormalities include many chromosome rearrangements (translocations, deletions and inversions) which lead to the formation of fusion genes. These fusion genes are often associated with particular forms of tumor, and detection of them may contribute significantly to the making of a diagnosis (The impact of translocations and gene fusions on cancer causation. Mitelman F, Johansson B, Mertens F., Nat Rev Cancer. 2007 April; 7 (4): 233-45). They are also often used as molecular markers for monitoring the efficacy of treatments and tracing the evolution of the disease, for example in acute leukemias (Standardized RT-PCR analysis of fusion gene transcripts from chromosome aberrations in acute leukemia for detection of minimal residual disease. Report of the BIOMED-1 Concerted Action: investigation of minimal residual disease in acute leukemia. van Dongen J J, Macintyre E A, Gabert J A, Delabesse E, Rossi V, Saglio G, Gottardi E, Rambaldi A, Dotti G, Griesinger F, Parreira A, Gameiro P, Diáz M G, Malec M, Langerak A W, San Miguel J F, Biondi A. Leukemia. 1999 December; 13 (12): 1901-28).

Today, the two main techniques for finding these fusion genes are cytogenetics and RT-PCR.

Cytogenetics consists of establishing the karyotype of the cancer cells for finding any abnormalities of number and/or of structure of the chromosomes. It has the advantage of providing an overall view of the whole genome. It is, however, of relatively low sensitivity, and its efficacy is strongly dependent on the percentage of tumor cells in the sample to be analyzed and the possibility of obtaining viable cell cultures. Another of its drawbacks is its low resolution, which does not allow certain rearrangements to be detected (in particular small inversions and deletions). Finally, certain tumors are associated with a major genomic instability that masks the pathognomonic genetic abnormalities. Analysis of the karyotypes is therefore often difficult and can only be carried out by personnel possessing excellent expertise.

The second technique, RT-PCR, is carried out starting from RNA extracted from the tumor cells. It has excellent sensitivity, far higher than cytogenetics. This sensitivity makes it the reference technique for analyzing biological samples where the percentage of tumor cells is low, which makes it possible to monitor the efficacy of the treatments or anticipate any relapses very early. However, its main limitation is connected with the fact that it is extremely difficult to multiplex analyses of this type. As each translocation generally has to be found by a specific test, only a few recurrent fusions among the numerous that are now known are investigated in routine analysis laboratories.

Therefore there is now a need for a specific, sensitive test, which is also simple and quick to implement, allowing accurate diagnosis of a cancer, notably a solid tumor (sarcoma or carcinoma) or a leukemia.

### BRIEF SUMMARY OF THE INVENTION

Surprisingly, the inventors have succeeded in developing an assay for diagnosing a specific cancer, notably a solid tumor (for example sarcoma or carcinoma) or a leukemia. This assay allows simultaneous investigation for a very large number of chromosome rearrangements in many forms of cancer, both economically and quickly (the assay may be carried out in one day). Moreover, its simplicity of use allows a person who has mastered the conventional techniques of molecular biology to perform all of the steps without special training, using equipment already available in most routine diagnostic laboratories.

The invention therefore relates to a molecular assay notably comprising an RT-MLPA step (Reverse Transcriptase Ligation-Dependent Probe Amplification), performed in multiplex mode. The multiplex mode gives a saving of time, as it is quicker than several monoplex modes, and is economically advantageous. It also allows simultaneous investigation for a much higher number of abnormalities than the techniques currently available.

The present invention therefore relates to a method for diagnosing a cancer in a subject, comprising a step of RT-MLPA on a biological sample obtained from said subject, using at least one pair of probes selected from:
probes SEQ ID NOs: 1 to 25, 30, 31 and 113 to 120,
probes SEQ ID NOs: 374 to 405, and
probes SEQ ID NOs: 524 to 559,
   each of the probes being fused, at one end at least, with a priming sequence. Preferably, said pair of probes consists of two probes that hybridize side by side during the RT-MLPA step.

Preferably, the present invention relates to a method for diagnosing a cancer in a subject, comprising an RT-MLPA step carried out on a biological sample obtained from said subject using the probes SEQ ID NOs: 1 to 25, 30, 31 and 113 to 120, and/or using the probes SEQ ID NOs: 374 to 405, and/or at least the probes SEQ ID NOs: 524 to 559, each of the probes being fused, at one end at least, with a priming sequence.

The present invention also relates to a kit comprising at least the probes SEQ ID NOs: 1 to 25, 30, 31 and 113 to 120, and/or the probes SEQ ID NOs: 374 to 405, and/or at least the probes SEQ ID NOs: 524 to 559, preferably further comprising the probes SEQ ID NOs: 26 to 29, 66 to 112 and 121 to 219, and/or the probes SEQ ID NOs: 438 to 480, and/or the probes SEQ ID NOs: 616 to 674, and/or the probes SEQ ID NOs: 734 to 741, and/or the probes SEQ ID NOs: 750 to 774, each of the probes being fused, at one end at least, with a priming sequence. Preferably, the kit comprises at least the probes SEQ ID NOs: 35 to 59, 64, 65 and 267 to 274, and/or the probes SEQ ID NOs: 406 to 437, and/or the probes SEQ ID NO: 560 to 595, preferably it further comprises the probes SEQ ID NOs: 60 to 63, 220 to 266 and 275 to 373, and/or the probes SEQ ID NOs: 675 to 733, and/or the probes SEQ ID NOs: 775 to 799, and/or the probes SEQ ID NOs: 742 to 749.

“MLPA” means Multiplex Ligation-Dependent Probe Amplification, which allows simultaneous amplification of several targets of interest contiguous with one another, using one or more specific probes. This technique is very advantageous, in the context of the present invention, for determining the presence of translocations, which are frequent in malignant tumors.

“RT-MLPA” means Multiplex Ligation-Dependent Probe Amplification preceded by Reverse Transcription (RT), which makes it possible, in the context of the present invention, to start from the RNA of a subject to amplify and characterize the fusion genes of interest.

“Subject” means an individual who is healthy or may have a cancer or for screening, diagnosis or monitoring.

“Biological sample” means a sample containing biological material. More preferably, it means any sample containing RNA. This sample may be obtained from biological sampling carried out on a living being (human patient, animal, plant). Preferably, the biological samples according to the invention are selected from whole blood, bone marrow and a biopsy, obtained from a subject, notably human.

“Sensitivity” means the proportion of positive tests in subjects with cancers and who have the abnormalities under investigation.

“Specificity” means the proportion of negative tests in subjects without cancers and who do not have the abnormalities under investigation.

“Cancer” means a disease characterized by an abnormally high level of cellular proliferation within a normal tissue of the organism, so that the latter's survival is threatened. According to the invention, the cancer is preferably selected from leukemias, sarcomas and carcinomas. Leukemias are cancers of the cells of the bone marrow, and of the lymphoid and myeloid systems. Sarcomas are malignant tumors that have developed at the expense of common extraskeletal connective tissue such as adipose tissue, muscle tissue, vessels and the peripheral nervous system. Carcinomas are malignant tumors that have developed at the expense of epithelial tissue.

“Probe” means a nucleic acid sequence with a length of between 15 and 40 nucleotides, preferably between 20 and 25 nucleotides, and complementary to a cDNA sequence from an RNA of the subject (endogenous). It is therefore capable of hybridizing to said cDNA sequence from an RNA of the subject.

“Priming sequence” means a nucleic acid sequence with a length of between 15 and 30 nucleotides, preferably between 20 and 25 nucleotides, and not complementary to the cDNA sequences from the subject's RNA. Therefore it is not complementary to the cDNA corresponding to the endogenous RNA. Therefore it cannot hybridize to said cDNA sequences. Preferably, the priming sequence is selected from the sequences SEQ ID NO: 33 and SEQ ID NO: 34.

### BRIEF DESCRIPTION OF THE SEVERAL VIEWS OF THE DRAWINGS

FIG. 1A shows the intron-exon structure of the genes involved in the translocations. FIG. 1B shows the points of breaks that may lead to expression of functional chimeric proteins. FIG. 1C shows the exons of the two genes juxtaposed after splicing of the hybrid transcript. FIG. 1D shows a set of probes.

FIG. 2A-2F: Scheme of RT-MLPA fusion gene: (FIG. 2A) translocation scheme; FIG. 2B) reverse transcription scheme; FIG. 2C) hybridization scheme; FIG. 2D) ligation scheme; E) PCR scheme; and FIG. 2F) results.

FIG. 3A-3B: Identification of the translocations in leukemia cases. Identification of the partners of the MLL gene of samples No. 14 (A) and 15 (B) in FIG. 2.

FIG. 4(1)-4(2): Table of probes.

FIG. 5(1)-5(6): Table of probes.

FIG. 6A-6B: Identification of the translocations in cases of leukemia (A) and of solid tumors (B).

FIG. 7: Table of probes.

FIG. 8(1)-8(2): Table of probes.

FIG. 9(1)-9(2): Table of probes.

FIG. 10A-10C: Identification of the fusion genes: FIG. 10A) fusion genes in leukemia cases; FIG. 10B) fusion genes in sarcoma cases; FIG. 10C) fusion genes in carcinoma cases.

FIG. 11: Identification of the fusion genes in the case of leukemias.

FIG. 12: Identification of the fusion genes in the case of sarcomas and carcinomas.

FIG. 13(1)-13(3): Table of probes.

FIG. 14: Table of probes.

FIG. 15: Table of probes.

FIG. 16: Identification of the fusion genes targeted in example 2.

### DETAILED DESCRIPTION OF THE INVENTION

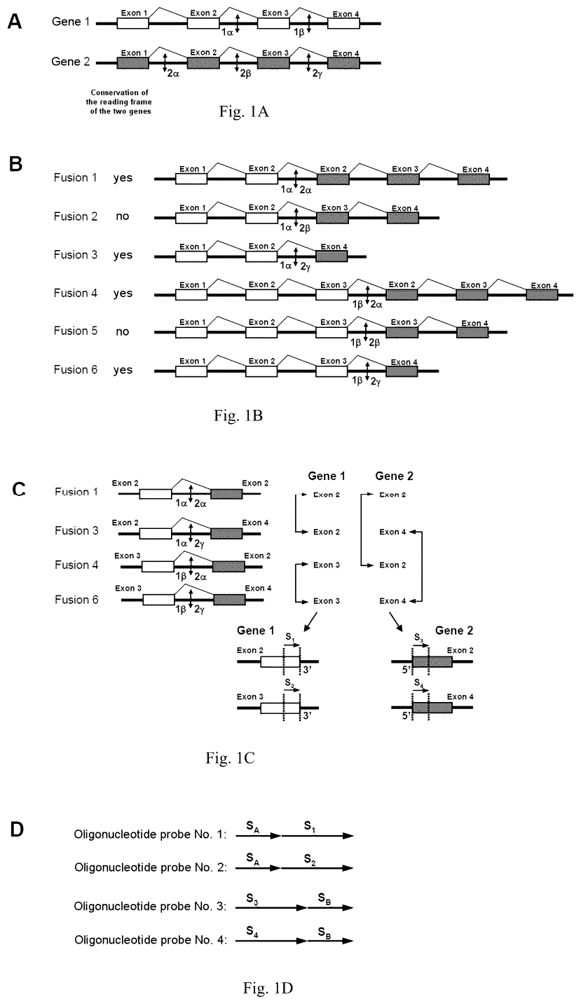

The inventors have identified specific probes for each type of translocation observed in certain cancers. This identification is based on analysis of the intron-exon structure of the genes involved in the translocations, as is shown in FIG. 1A-1D. In particular, the points of breaks that may lead to expression of functional chimeric proteins are sought (FIG. 1B). Based on these results, DNA sequences with 25 to 40 base pairs are defined, precisely corresponding to the 5′ and 3′ ends of the exons of the two genes juxtaposed after splicing of the hybrid transcripts (FIG. 1C). A set of probes is then defined as follows: a priming sequence (S_{A} in FIG. 1A-1D) with about twenty base pairs is added at 5′ of all the probes complementary to the exons of the genes forming the 5′ portion of the fusion transcripts. A second priming sequence (S_{B} in FIG. 1A-1D), also with about twenty base pairs but different from S_{A}, is added to the 3′ ends of all the probes complementary to the exons of the genes forming the 3′ portion of the fusion transcripts (FIG. 1D). These probes are then combined in a mixture, and contain all the elements required for detecting one or more fusion transcripts, produced by one or more translocations.

The probes used in the invention are therefore capable of hybridizing either to the last nucleotides of the last exon at 5′ of the translocation, or to the first nucleotides of the first exon at 3′ of the translocation. Preferably, the probes used in the invention that are capable of hybridizing to the first nucleotides of the first exon at 3′ of the translocation are phosphorylated at 5′ before they are used.

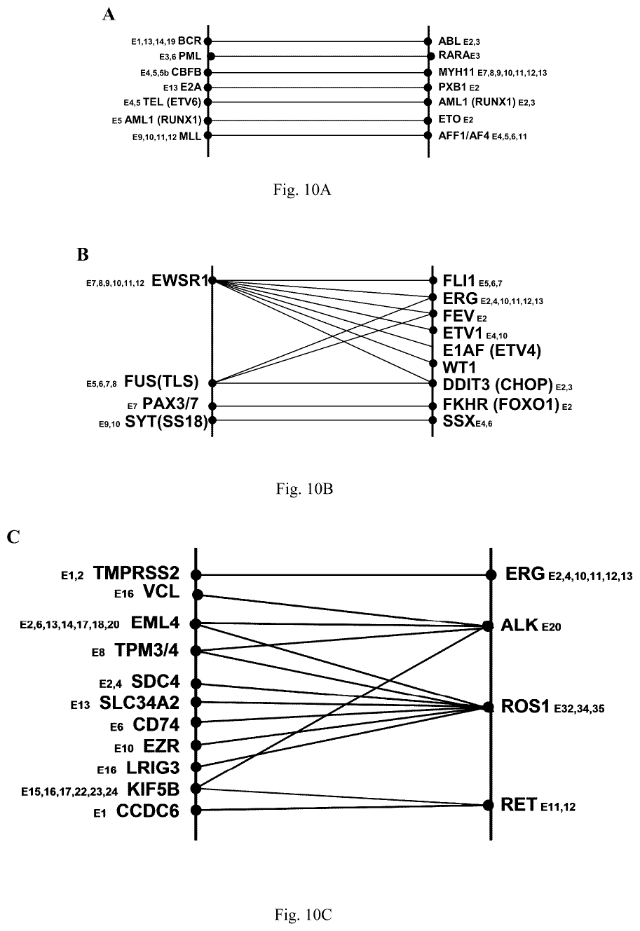

The various translocations identified according to the present invention are illustrated in FIGS. 10A-10C to 12.

The probes according to the invention may be the sequences SEQ ID NOs: 1 to 25,30, 31 and 113 to 120, optionally combined with the probes SEQ ID NOs: 26 to 29, 66 to 112 and 121 to 219 and/or with the probes SEQ ID NOs: 616 to 674.

The probes according to the invention may be the sequences SEQ ID NOs: 374 to 405, optionally combined with the probes SEQ ID NOs: 438 to 480 and/or at least with the probes SEQ ID NOs: 750 to 774.

The probes according to the invention may be the sequences SEQ ID NOs: 524 to 559, optionally combined with the probes SEQ ID NOs: 438 to 480 and/or at least with the probes SEQ ID NOs: 374 to 405 and/or with the probes SEQ ID NOs: 734 to 741.

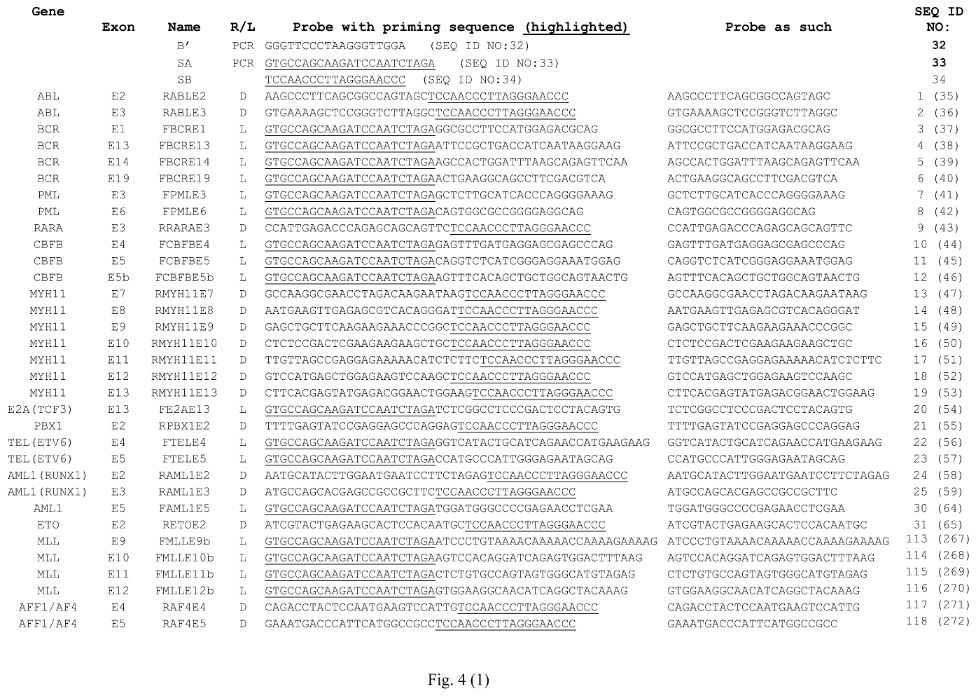

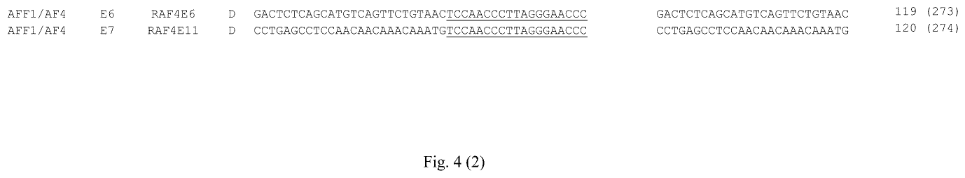

FIGS. 4(1)-4(2), 5(1)-5(6), and 7 to 9(1)-9(2) give details of the name of each probe, their position (i.e. whether they are at 5′ (L) or at 3′ (R) of the abnormal junction), their structure (presence or absence of a priming sequence, and characteristics of this sequence), as well as the gene and the exon to which each probe hybridizes.

Preferably, each of the probes SEQ ID NO: 3 to SEQ ID NO: 8; SEQ ID NO: 10 to SEQ ID NO: 12; SEQ ID NO: 20, SEQ ID NO: 22, SEQ ID NO: 23, SEQ ID NO: 26 and SEQ ID NO: 30 is fused at 5′ with a priming sequence, and

each of the probes SEQ ID NO: 1, SEQ ID NO: 2, SEQ ID NO: 9, SEQ ID NO: 13 to SEQ ID NO: 19; SEQ ID NO: 21, SEQ ID NO: 24, SEQ ID NO: 25, SEQ ID NO: 27 to SEQ ID NO: 29; and SEQ ID NO: 31 is fused at 3′ with a different priming sequence.

Preferably, the probes fused with the priming sequences usable according to the invention are the sequences SEQ ID NOs: 35 to 59, 64, 65 and 267 to 274, optionally combined with the probes SEQ ID NOs: 60 to 63, 220 to 266 and 275 to 373 and/or with the probes SEQ ID NOs: 675 to 733.

Preferably, the probes fused with the priming sequences usable according to the invention are the sequences SEQ ID NOs: 406 to 437, optionally combined with the probes SEQ ID NOs: 481 to 523 and/or with the probes SEQ ID NOs: 775 to 799.

Preferably, the probes fused with the priming sequences usable according to the invention are the sequences SEQ ID NOs: 560 to 595, optionally combined with the probes SEQ ID NOs: 481 to 523 and/or with the probes SEQ ID NOs: 742 to 749.

The method of diagnosis according to the invention is carried out with at least one pair of probes selected from:
probes SEQ ID NOs: 1 to 25, 30, 31 and 113 to 120,
probes SEQ ID NOs: 374 to 405, and
probes SEQ ID NOs: 524 to 559,
   each of the probes being fused, at one end at least, with a priming sequence.

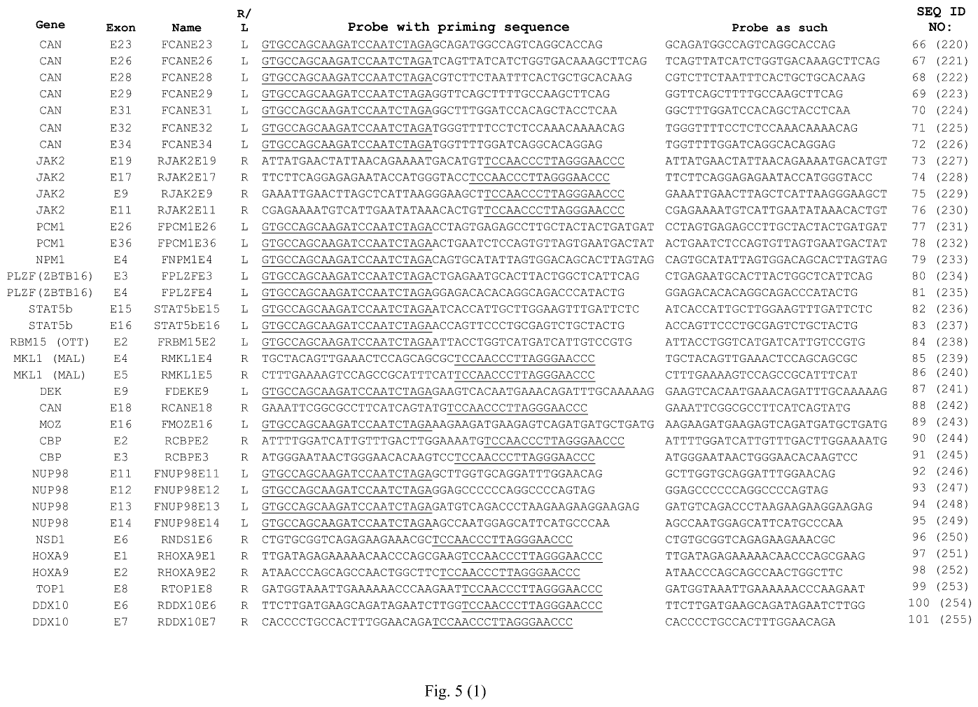

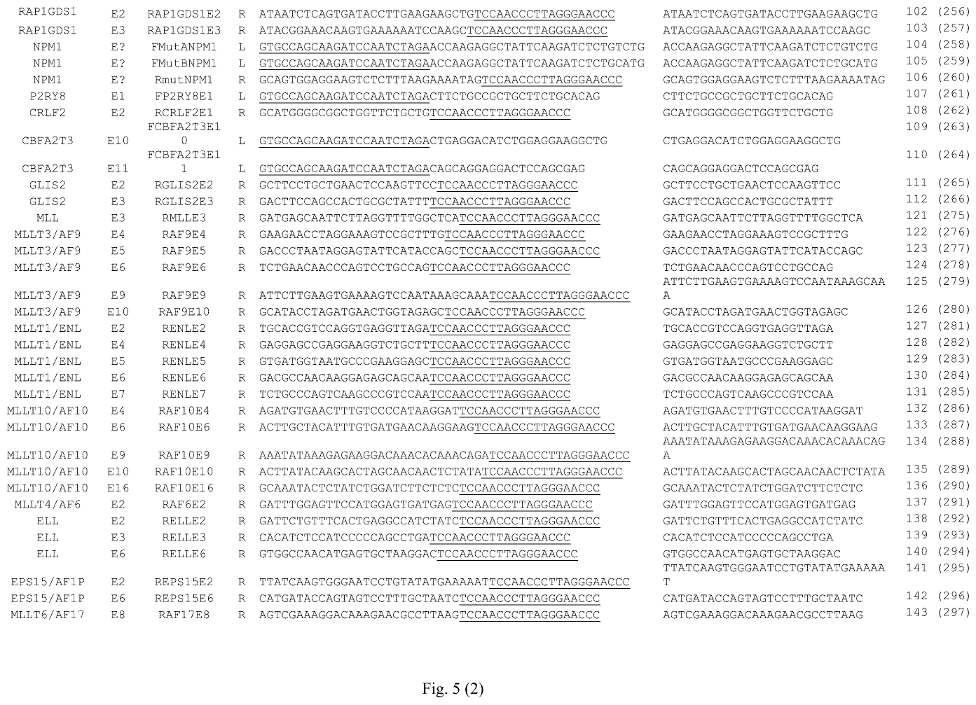

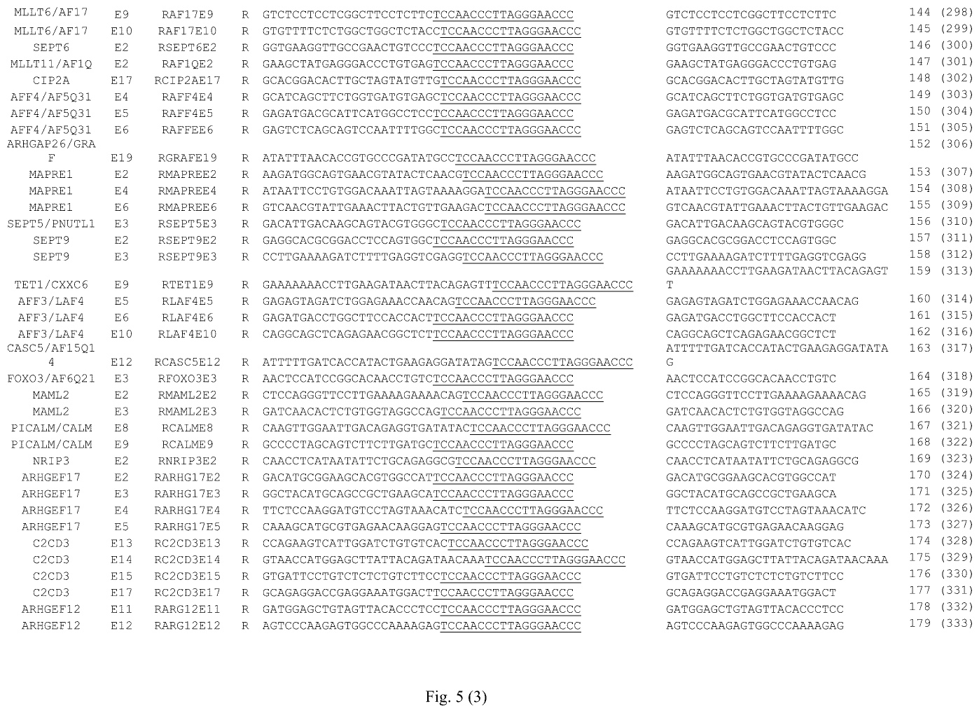

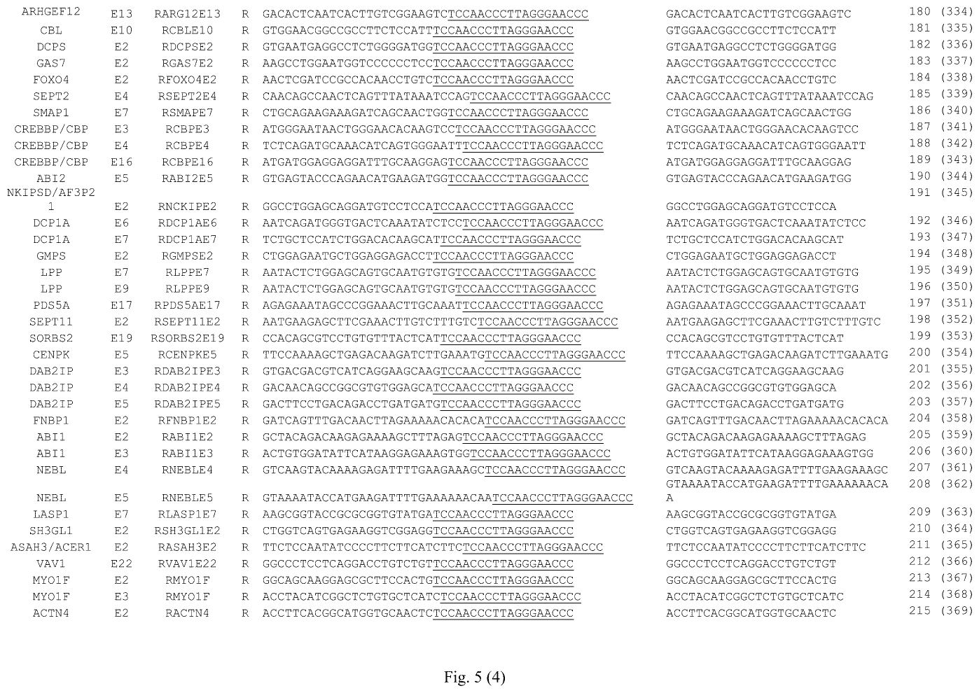

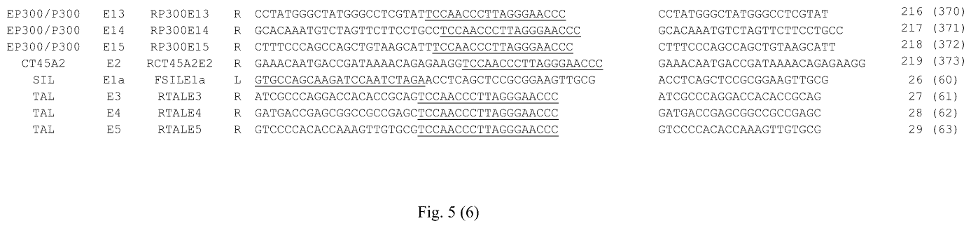

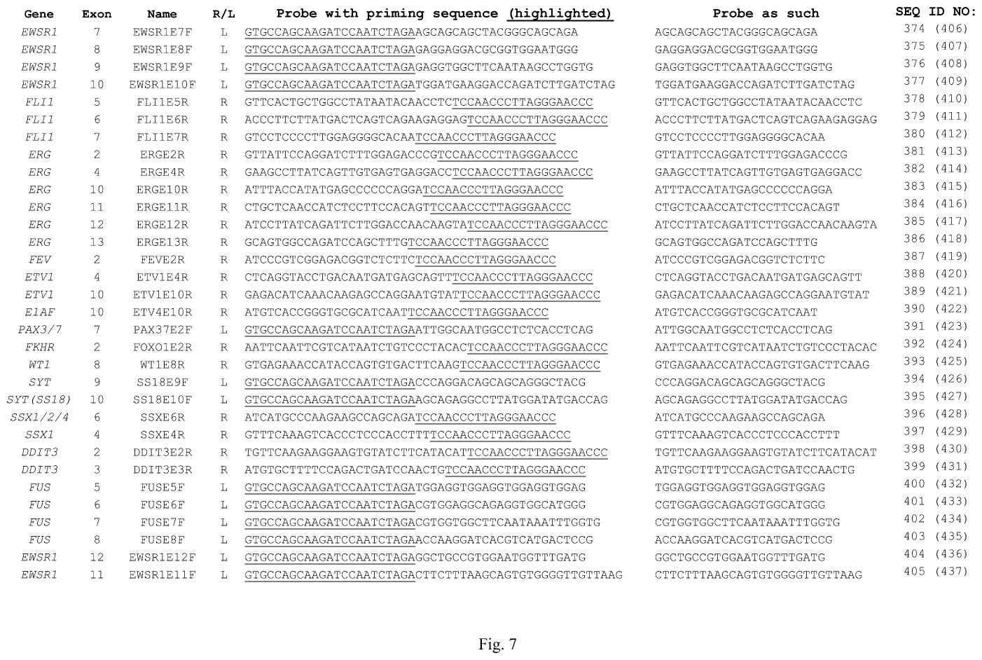

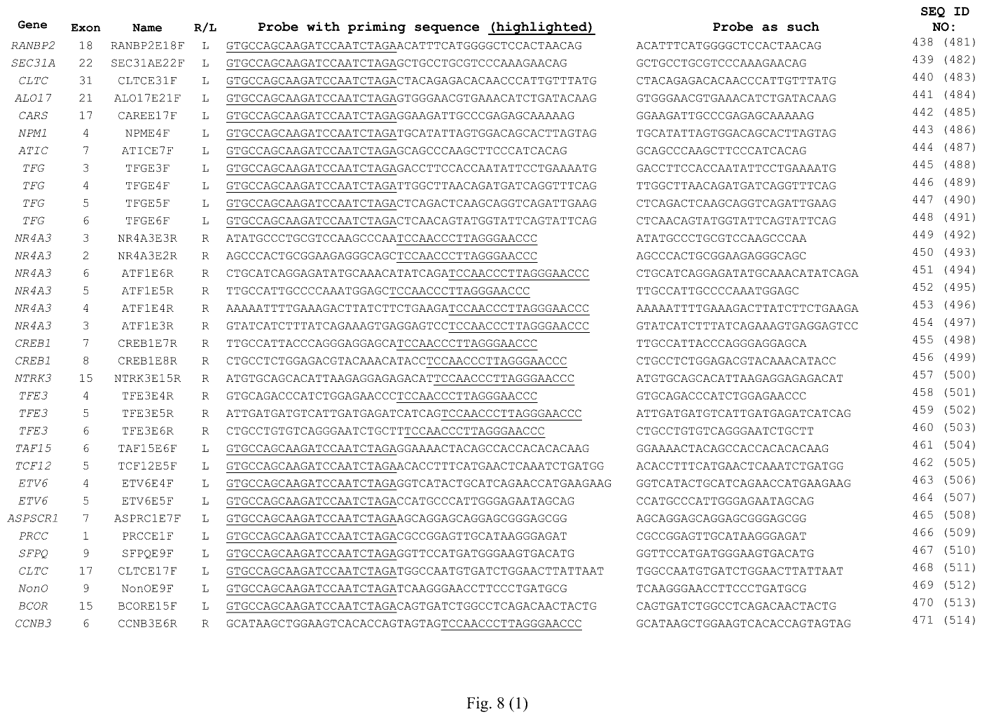

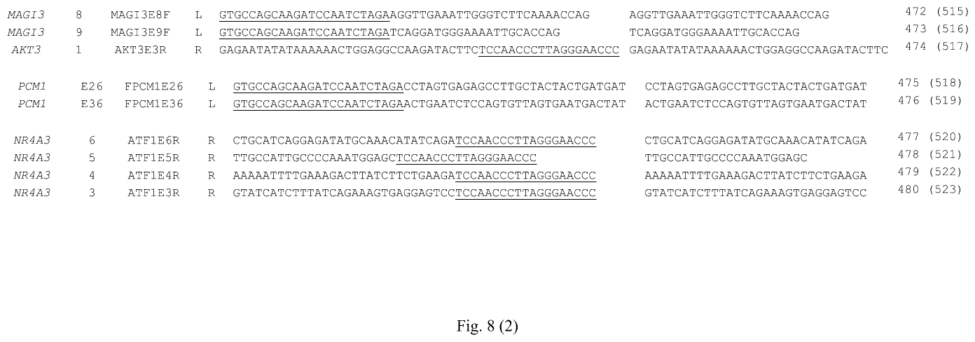

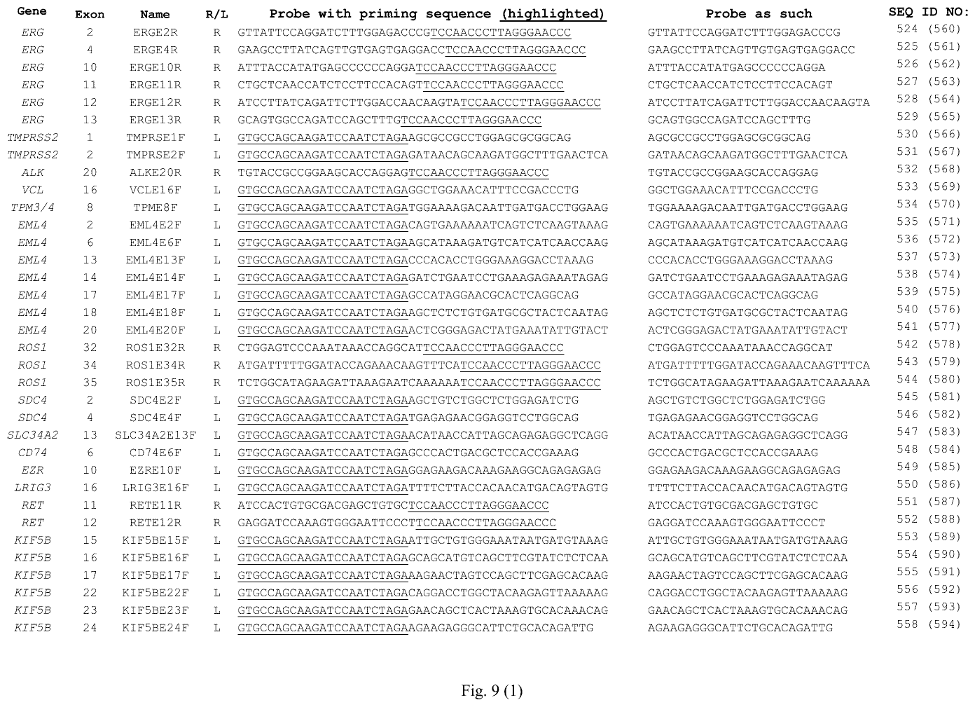

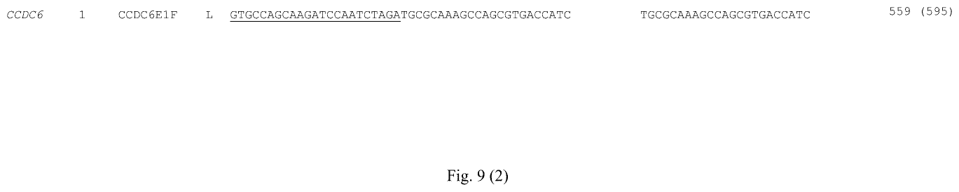

The pair of probes may be selected from the probes with a specific sequence described above, and as is explained in FIGS. 4(1)-4(2), 7 and 9(1)-9(2). “Pair of probes” means a set of two probes, one being situated at 5′ (“L” in FIGS. 4(1)-4(2), FIGS. 5(1)-5(6), FIG. 7, FIGS. 8(1)-8(2), and FIGS. 9(1)-9(2)) of the translocation or gene mutation, the other being situated at 3′ (“R” in FIGS. 4(1)-4(2), FIGS. 5(1)-5(6), FIG. 7, FIGS. 8(1)-8(2), and FIGS. 9(1)-9(2)) of the translocation or gene mutation.

Preferably, the method of diagnosis according to the invention relates to the diagnosis of leukemias, and comprises an RT-MLPA step carried out on a biological sample obtained from said subject using the probes SEQ ID NOs: 1 to 25, 30, 31 and 113 to 120, each of the probes being fused, at one end at least, with a priming sequence.

The leukemias are preferably selected from B-cell acute lymphoblastic leukemias, T-cell acute lymphoblastic leukemias, acute myeloblastic leukemias, chronic myeloid leukemias, lymphomas and myelomas.

Preferably, in this case, the method of diagnosis according to the invention also uses the probes SEQ ID NOs: 26 to 29, 66 to 112 and 121 to 219, and/or the probes SEQ ID NOs: 616 to 674, for the RT-MLPA step, each of the probes being fused, at one end at least, with a priming sequence. Thus, in this case, a mixture of probes SEQ ID NOs: 1 to 25, 30, 31 and 113 to 120, and SEQ ID NOs: 26 to 29, 66 to 112 and 121 to 219 and/or SEQ ID NOs: 616 to 674 is used, each of the probes being fused, at one end at least, with a priming sequence.

Preferably, the invention relates to a diagnostic kit for leukemias comprising the probes SEQ ID NOs: 1 to 25, 30, 31 and 113 to 120, and 26 to 29, 66 to 112 and 121 to 219, and/or SEQ ID NOs: 616 to 674, each of the probes being fused, at one end at least, with a priming sequence. Preferably, said kit comprises the probes SEQ ID NOs: 35 to 59, 64, 65 and 267 to 274, and preferably also probes SEQ ID NOs: 60 to 63, 220 to 266 and 275 to 373, and/or preferably also probes SEQ ID NOs: 675 to 733.

Preferably, the method of diagnosis according to the invention relates to the diagnosis of sarcomas, and comprises a step of RT-MLPA in a biological sample obtained from said subject using the probes SEQ ID NOs: 374 to 405, each of the probes being fused, at one end at least, with a priming sequence.

The sarcomas are preferably selected from Ewing sarcomas, rhabdomyosarcomas, desmoplastic round-cell tumors, synovial sarcomas and myxoid liposarcomas.

Preferably, in this case, the method of diagnosis according to the invention also uses the probes SEQ ID NOs: 438 to 480 and/or probes SEQ ID NOs: 750 to 774, for the

RT-MLPA step, each of the probes being fused, at one end at least, with a priming sequence. Thus, in this case, a mixture of probes SEQ ID NOs: 374 to 405, and 438 to 480 and/or 750 to 774, is used, each of the probes being fused, at one end at least, with a priming sequence.

Preferably, the invention relates to a diagnostic kit for sarcomas comprising the probes SEQ ID NOs: 374 to 405, each of the probes being fused, at one end at least, with a priming sequence. Preferably, said kit comprises the probes SEQ ID NOs: 406 to 437, and preferably also probes SEQ ID NOs: 481 to 523, and/or preferably also probes SEQ ID NOs: 775 to 799.

Preferably, the method of diagnosis according to the invention relates to the diagnosis of carcinomas, and comprises a step of RT-MLPA in a biological sample obtained from said subject using the probes SEQ ID NOs: 524 to 559, each of the probes being fused, at one end at least, with a priming sequence.

The carcinomas are preferably selected from nonsmall cell bronchopulmonary carcinomas, prostatic adenocarcinomas, carcinomas of the kidney, thyroid and breast.

Preferably, in this case, the method of diagnosis according to the invention also uses the probes SEQ ID NOs: 438 to 480 and/or probes SEQ ID NOs: 734 to 741, for the RT-MLPA step, each of the probes being fused, at one end at least, with a priming sequence. Thus, in this case, a mixture of probes SEQ ID NOs: 524 to 559, and 438 to 480 and/or 734 to 741, is used, each of the probes being fused, at one end at least, with a priming sequence.

Preferably, the invention relates to a diagnostic kit for carcinomas comprising the probes SEQ ID NOs: 524 to 559, each of the probes being fused, at one end at least, with a priming sequence. Preferably, said kit comprises the probes SEQ ID NOs: 560 to 595, and preferably also probes SEQ ID NOs: 481 to 523, and/or preferably also probes SEQ ID NOs: 742 to 749.

Preferably the method of the invention uses a biological sample selected from whole blood, bone marrow and a biopsy obtained from the subject.

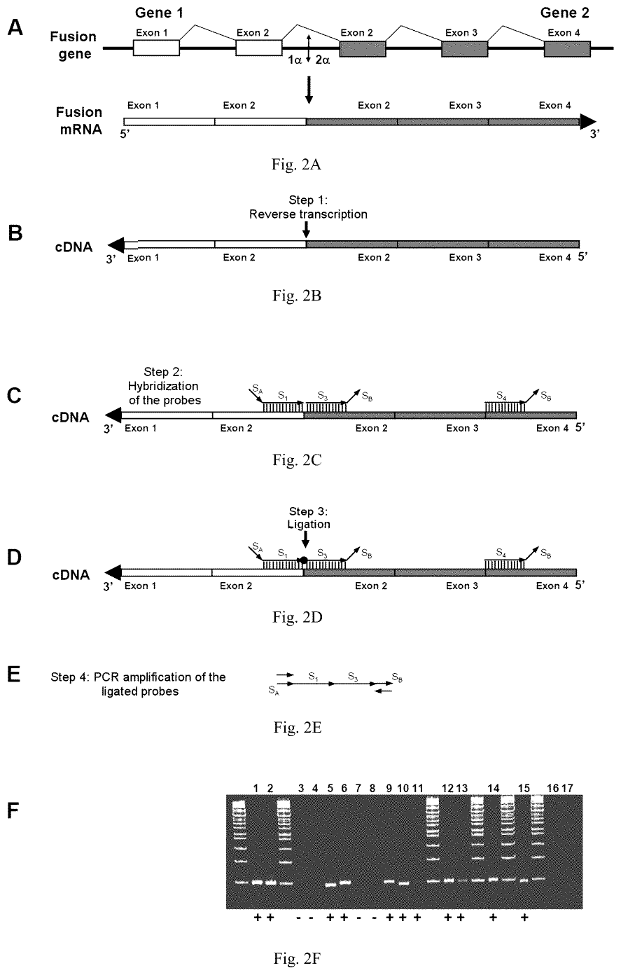

The RT-MLPA step is derived from MLPA (Multiplex Ligation-Dependent Probe Amplification), notably described in patent U.S. Pat. No. 6,955,901. It allows simultaneous detection and assay of a large number of different oligonucleotide sequences. The principle is as follows (see FIG. 2A-2F): RNA extracted from tumor tissue is first converted to complementary DNA (cDNA) by reverse transcription. This cDNA is then incubated with a mixture of suitable probes, and each can then hybridize to the sequences of the exons to which they correspond. If one of the fusion transcripts sought is present in the sample, two probes will bind side by side to the corresponding cDNA. A ligation reaction is then performed using an enzyme with DNA ligase activity, which establishes a covalent bond between the two contiguous probes. A PCR reaction (Polymerase Chain Reaction) is then carried out, using primers corresponding to the priming sequences (SA and SB in FIG. 2A-2F), which makes it possible to amplify the two ligated probes specifically. Obtaining an amplification product after the RT-MLPA step indicates that one of the translocations sought is present in the sample analyzed.

Preferably, the RT-MLPA step used in the method according to the invention comprises at least the following steps:
a) extraction of RNA from the biological sample from the subject;
b) conversion of the RNA extracted in a) to cDNA by reverse transcription;
c) incubation of the cDNA obtained in b) with at least one pair of probes selected from:
   probes SEQ ID NOs: 1 to 25, 30, 31 and 113 to 120,
   probes SEQ ID NOs: 374 to 405, and
   probes SEQ ID NOs: 524 to 559,
      each of the probes being fused, at one end at least, with a priming sequence. Preferably, in this step c), mixtures of probes as described above are used.
      Preferably, in this step c), the cDNA obtained in b) is incubated with at least the probes SEQ ID NOs: 1 to 25, 30, 31 and 113 to 120, and/or with at least the probes SEQ ID NOs: 374 to 405, and/or with at least the probes SEQ ID NOs: 524 to 559, each of the probes being fused, at one end at least, with a priming sequence;
d) addition of a DNA ligase to the mixture obtained in c), in order to establish a covalent bond between two contiguous probes;
e) PCR amplification of the covalently bound contiguous probes obtained in d).

Typically, extraction of RNA from the biological sample according to step a) is performed by the conventional techniques that are familiar to a person skilled in the art. For example, this extraction may be performed by cellular lysis of the cells obtained from the biological sample. This lysis may be of a chemical, physical or thermal nature. This cellular lysis is generally followed by a purification step for separating and concentrating the nucleic acids from other cellular debris. Commercial kits of the QIAGEN and Zymo Research type, or else those marketed by Invitrogen, may be used for carrying out step a). Of course, the various relevant techniques differ depending on the nature of the biological sample being assayed. The knowledge of a person skilled in the art allows him to easily adapt these steps of lysis and purification to said biological sample being tested.

Preferably, the RNA extracted in step a) is then converted by reverse transcription to cDNA; this is step b) (see FIG. 2B). This step b) may be carried out using any technique of reverse transcription known from the prior art. It may notably be done using reverse transcriptase marketed by Qiagen, Promega or Ambion, according to the classical conditions for use, or else using M-MLV Reverse Transcriptase from Invitrogen.

Preferably, the cDNA obtained in step b) is then incubated with at least the probes SEQ ID NOs: 1 to 25, 30, 31 and 113 to 120, and/or with at least the probes SEQ ID NOs: 374 to 405, and/or with at least the probes SEQ ID NOs: 524 to 559, each of the probes being fused, at one end at least, with a priming sequence. This is step c) of hybridization of the probes (see FIG. 2C). In fact, the probes, which are complementary to a portion of cDNA, will hybridize to this portion if the latter is present in the cDNA. As is shown in FIG. 2C, owing to their sequence, the probes will therefore hybridize:
either to the portion of cDNA corresponding to the last nucleotides of the last exon at 5′ of the translocation. They are then “F” or “Forward” probes, also called “L” or “Left”;
or to the portion of cDNA corresponding to the first nucleotides of the first exon at 3′ of the translocation. They are then “R” or “Reverse” probes, also called “R” or “Right”. At the end of step c), the probes hybridized to cDNA are contiguous, if and only if the translocation has taken place.
   This step c) is typically carried out by incubating the cDNA and the mixture of probes at a temperature between 90° C. and 100°° C., for a time of from 1 to 5 minutes, and then leaving them to incubate for at least 1 h, preferably 16 h, at a temperature of about 60° C. It may be carried out using the commercial kit sold by the company MRC-Holland (SALSA MLPA Buffer).

At the end of step c), a DNA ligase is typically added to bind covalently only the contiguous probes; this is step d) (see FIG. 2D). The DNA ligase is notably ligase 65, sold by MRC-Holland, Amsterdam, the Netherlands (SALSA Ligase-65). This step d) is typically carried out using the kits Lig-5a, Lig-10 or Lig-50 from MRC-Holland, Amsterdam, the Netherlands. It is typically carried out by incubating the DNA ligase and the mixture obtained in step c) at a temperature between 50° C. and 60° C., for a time of from 10 to 20 minutes, and then for a time of from 2 to 10 minutes at a temperature between 95° C. and 100° C.

At the end of step d), each pair of contiguous probes L and R is bound covalently, and the priming sequence of each probe is still present at 5′ and at 3′.

Preferably, the method also comprises a step e) of PCR amplification of the covalently bound contiguous probes obtained in d) (see FIG. 2E). This PCR step is carried out using a primer pair, one of the primers being identical to the priming sequence at 5′, and the other primer being complementary to the priming sequence at 3′. Preferably, the PCR amplification in step e) is carried out using the primers SEQ ID NOs: 32 and 33, preferably one being labeled at its 5′ end with a biotin, in order to allow step (f). PCR is typically carried out using commercial kits, such as the ready-to-use kits sold by Eurogentec (Red‘y’Star Mix). Typically, PCR takes place in a first phase of initial denaturation at a temperature between 90° C. and 100° C., typically about 94° C., for a time of from 5 to 8 minutes; then a second phase of amplification comprising several cycles, typically 35 cycles, each cycle comprising 30 seconds at 94° C., then 30 seconds at 58° C., then 30 seconds at 72° C.; and a last phase of return to 72° C. for about 4 minutes. At the end of the PCR, the amplicons are stored, preferably at 4° C.

Typically, the primers usable in step e) of PCR are as follows:

| SEQ ID NO: | Primer |
|---|---|
| 596 | CMV Forward: |
| | CGC AAA TGG GCG GTA GGC GTG |
| 597 | CMV Reverse: |
| | CGC CAT CCA CGC TGT TTT G |
| 598 | pcDNA3 Forward: |
| | GGC TAA CTA GAG AAC CCA CTG |
| 599 | pcDNA3 Reverse: |
| | GGC AAC TAG AAG GCA CAG TC |
| 600 | pCEP Forward: |
| | AGA GCT CGT TTA GTG AAC CG |
| 601 | pCEP Reverse: |
| | GTG GTT TGT CCA AAC TCA TC |
| 602 | pEGFPC1 Forward: |
| | GAT CAC TCT CGG CAT GGA C |
| 603 | pEGFPC1 Reverse: |
| | CAT TTT ATG TTT CAG GTT CAG GG |
| 604 | pEGFPN1 Forward: |
| | GTC GTA ACA ACT CCG CCC |
| 605 | pEGFPN1 Reverse: |
| | GTC CAG CTC GAC CAG GAT G |
| 606 | pGex Forward: |
| | ATA GCA TGG CCT TTG CAG G |
| 607 | pGex Reverse: |
| | GAG CTG CAT GTG TCA GAG G |
| 608 | pGL Forward: |
| | GTA TCT TAT GGT ACT GTA ACT G |
| 609 | pGL Reverse: |
| | CTT TAT GTT TTT GGC GTC TTC C |
| 610 | pShuttleCMV Forward: |
| | GGT CTA TAT AAG CAG AGC TG |
| 611 | pShuttleCMV Reverse: |
| | GTG GTA TGG CTG ATT ATG ATC AG |
| 32 | Reverse: |
| | GGGTTCCCTAAGGGTTGGA |
| | (complementary to the priming sequence |
| | SEQ ID NO: 34) |
| 33 | Forward: |
| | GTGCCAGCAAGATCCAATCTAGA |

Preferably, the method according to the invention comprises a step f) of analysis of the results of the PCR in step e), preferably by pyrosequencing. For this purpose, a pyrosequencer may be used, such as the PyroMark Q24 pyrosequencer sold by the company Qiagen, using the commercial kit Pyromark Gold Q24 Reagent and one of the priming oligonucleotides.

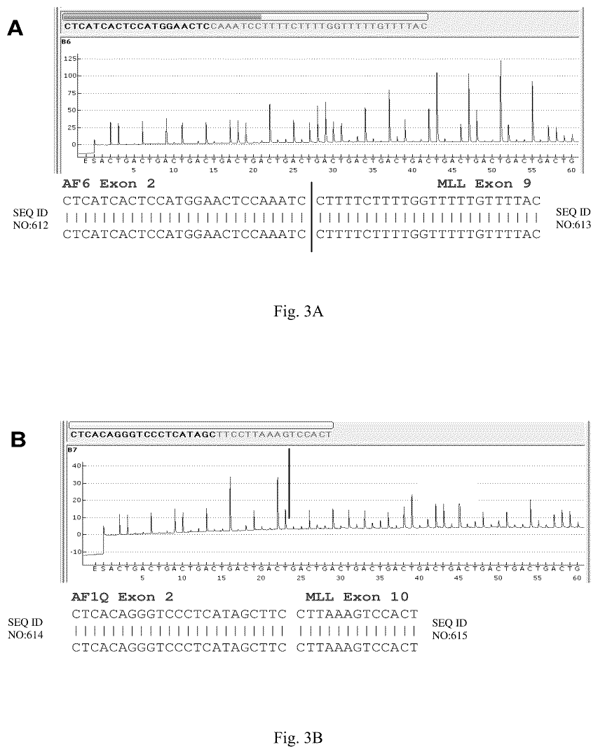

This analysis step allows immediate reading of the result, and indicates directly whether the sample from the subject bears a specific translocation, whether or not identified. This is notably demonstrated in FIG. 3A-3B.
In fact, in step f), if, for a biological sample from a subject, PCR amplification is obtained in step e) following hybridization to a pair of probes, then the subject has the cancer connected with the chromosome rearrangement corresponding to the pair of probes identified.

The various sequences mentioned in the present invention are summarized in the following table:

| SEQ ID NO: | Identification |
|---|---|
| 1-25, 30, 31 and 113 to 120 | Probes as such, notably for diagnosis of |
| | leukemias |
| 32 | Reverse PCR primer |
| 33 | Forward PCR primer and priming |
| | sequence at 5′ |
| 34 | Priming sequence at 3′ |
| 35-59, 64, 65 and 267-274 | Probes SEQ ID NO: 1-25, 30, 31 and 113 |
| | to 120 fused with the priming sequence |
| 26-29, 66-112 and 121-219 | Probes as such, notably for diagnosis of |
| | leukemias |
| 60-63, 220-266 and 275-373 | Probes SEQ ID NO: 26-29, 66-112 and |
| | 121-219 fused with the priming sequence |
| 374-405 | Probes as such, notably for diagnosis of |
| | sarcomas |
| 406-437 | Probes SEQ ID NO: 374-405 fused with |
| | the priming sequence |
| 438-480 | Probes as such, notably for diagnosis of |
| | solid tumors |
| 481-523 | Probes SEQ ID NO: 438-480 fused with |
| | the priming sequence |
| 524-559 | Probes as such, notably for diagnosis of |
| | carcinomas |
| 560-595 | Probes SEQ ID NO: 524-559 fused with |
| | the priming sequence |
| 596-611 | PCR primers |
| 612-615 | Sequences in FIG. 3A and B |
| 616-674 | Probes as such, notably for diagnosis of |
| | leukemias |
| 675-733 | Probes SEQ ID NO: 616-674 fused with |
| | the priming sequence |
| 734-741 | Probes as such, notably for diagnosis of |
| | carcinomas |
| 742-749 | Probes SEQ ID NO: 734-741 fused with |
| | the priming sequence |
| 750-774 | Probes as such, notably for diagnosis of |
| | sarcomas |
| 775-799 | Probes SEQ ID NO: 750-774 fused with |
| | the priming sequence |

### FIG. 1A-1D: Scheme for Definition of the Probes.

FIG. 1A-1D shows specific probes for each type of translocation observed in certain cancers. FIG. 1A shows the intron-exon structure of the genes involved in the translocations. FIG. 1B shows the points of breaks that may lead to expression of functional chimeric proteins. FIG. 1C shows the exons of the two genes juxtaposed after splicing of the hybrid transcript. FIG. 1D shows a set of probes with a first priming sequence (S_{A}) added at 5′ end of all the probes complementary to the exons of the genes forming the 5′ portion of the fusion transcripts. It also shows a second priming sequence (S_{B}) different from SA added to the 3′ ends of all the probes complementary to the exons of the genes forming the 3′ portion of the fusion transcripts.

### FIG. 2A-2F: Scheme of RT-MLPA.

A) Example of translocation in the intron located between exon 2 of gene 1 and exon 2 of gene 2, leading to a fusion mRNA.
B) Step 1: reverse transcription of this fusion mRNA, to obtain a cDNA.
C) Step 2: incubation with the probes and hybridization of the latter to the complementary portions of cDNA. Probe S1 consists of a complementary sequence of the last nucleotides of exon 2 of gene 1 of cDNA, and probe S2 consists of a sequence complementary to the first nucleotides of exon 2 of gene 2 of cDNA.

Probe S1 is fused at 5′ with a priming sequence SA.

Probe S2 is fused at 3′ with a priming sequence SB.

Owing to the contiguity between exons 2 of gene 1 and of gene 2, probes S1 and S2 are side by side.
D) Step 3: ligation by a DNA ligase. The probes that are side by side are then bound. S1 and S2 thus form a continuous sequence, with SA and SB.
E) Step 4: PCR: using suitable primers, the bound probes are amplified. In this case, the primers used are the sequence SA, and the sequence complementary to SB (called B′).
F: analysis of the results obtained. The figures show an example of results obtained with the probes that bind to the translocations involving the MLL gene (characteristic of leukemias) on samples obtained from 15 patients (tracks 1 to 15). Tracks 16 and 17 are negative controls.

It can be seen that 11 tumors out of the 15 have a rearrangement of the MLL gene.

FIG. 3A-3B: Identification of the Translocations in Leukemia Cases. Identification of the partners of the MLL gene of samples No. 14 (A) and 15 (B) in FIG. 2A-2F. The PCR amplification products were analyzed by pyrosequencing.

Sequence A corresponds to a junction between the 9th exon of the MLL gene and the 2nd exon of the AF6 gene, indicating that the tumor had a translocation t (6;11) (q27;q23).

Sequence B corresponds to a junction between the 10th exon of the MLL gene and the 2nd exon of the AF1Q gene, indicating that the tumor had a translocation t (1;11) (q21;q23). These two translocations confirm the diagnosis of acute leukemia, and are associated with forms with a very poor prognosis.

FIG. 4(1)-4(2): Table of Probes SEQ ID NOs: 1 to 25, 30, 31 and 113 to 120 (Corresponding to the Probes Fused with the Priming Sequence SEQ ID NOs: 35 to 59, 64, 65 and 267 to 274).

The table shows the gene and the exon to which the probe hybridizes. The column “SEQ ID NOs: ” gives the sequence number of the probe as such, and in parentheses the sequence number of the probe fused with the priming sequence.

These probes are usable notably for diagnosis of leukemias.

FIG. 5(1)-5(6): Table of Probes SEQ ID NOs: 26 to 29, 66 to 112 and 121 to 219 (Corresponding to the Probes Fused with the Priming Sequence SEQ ID NOs: 60 to 63, 220 to 266 and 275 to 373).

The table shows the gene and the exon to which the probe hybridizes. The column “SEQ ID NOs:” gives the sequence number of the probe as such, and in parentheses the sequence number of the probe fused with the priming sequence.

These probes are usable notably for diagnosis of leukemias.

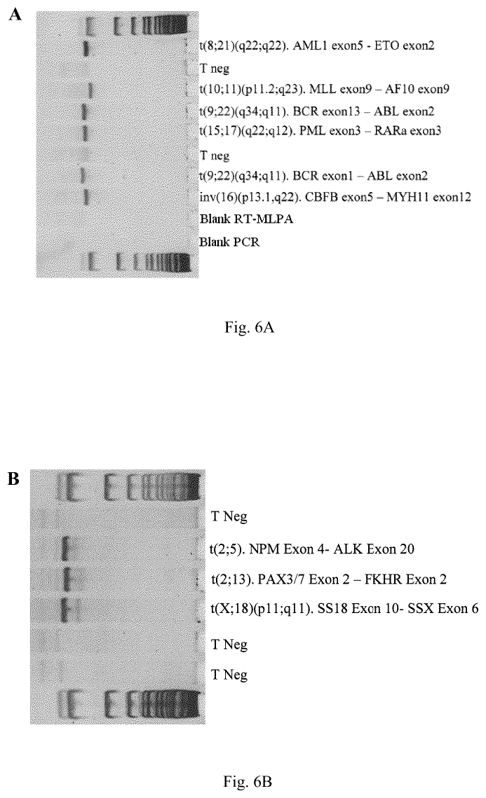

### FIG. 6A-6B: Identification of the Translocations in Cases of Leukemia (FIG. 6A) and of Solid Tumors (FIG. 6B).

The PCR amplification products were analyzed by pyrosequencing.

A) The translocations identified correspond to:
“AML1 exon5-ETO exon2”: AML2 (type 2 Acute myeloblastic leukemia)
“MLL exon9-AF10 exon9”: ALL (Acute lymphoblastic leukemia)
“BCR exon13-ABL exon2”: chronic myeloid leukemia
“PML exon3-RARa exon3”: AML3 (type 3 Acute myeloblastic leukemia)
“BCR exon1-ABL exon2”: ALL (Acute lymphoblastic leukemia); and
“CBFB exon5-MYH11 exon12”: AML4 (type 4 Acute myeloblastic leukemia).

B) The translocations identified correspond to a synovial sarcoma (SYT-SSX), to a rhabdomyosarcoma (PAX-FKHR), and to an anaplastic lymphoma (NPM-ALK).

The term “Tneg” denotes a control sample, obtained from a known tumor that does not have the genetic rearrangements being assayed.

FIG. 7: Table of Probes SEQ ID NOs: 374 to 405 (Corresponding to the Probes Fused with the Priming Sequence SEQ ID NOs: 406 to 437).

The table shows the gene and the exon to which the probe hybridizes. The column “SEQ ID NOs:” gives the sequence number of the probe as such, and in parentheses the sequence number of the probe fused with the priming sequence.

These probes are usable notably for diagnosis of sarcomas.

FIG. 8(1)-8(2): Table of Probes SEQ ID NOs: 438 to 480 (Corresponding to the Probes Fused with the Priming Sequence SEQ ID NOs: 481 to 523).

The table shows the gene and the exon to which the probe hybridizes. The column “SEQ ID NOs:” gives the sequence number of the probe as such, and in parentheses the sequence number of the probe fused with the priming sequence.

These probes are usable notably for diagnosis of sarcomas.

FIG. 9(1)-(2): Table of Probes SEQ ID NOs: 524 to 559 (Corresponding to the Probes Fused with the Priming Sequence SEQ ID NOs: 560 to 595).

The table shows the gene and the exon to which the probe hybridizes. The column “SEQ ID NOs:” gives the sequence number of the probe as such, and in parentheses the sequence number of the probe fused with the priming sequence.

These probes are usable notably for diagnosis of carcinomas.

### FIG. 10A-10C: Identification of the Fusion Genes.

FIG. 10A) Fusion genes in leukemia cases. The fusion transcripts hybridize to the probes SEQ ID NOs: 1 to 25, 30, 31 and 113 to 120.

FIG. 10B) Fusion genes in sarcoma cases. The fusion transcripts hybridize to the probes SEQ ID NOs: 374 to 405.

FIG. 10C) Fusion genes in carcinoma cases. The fusion transcripts hybridize to the probes SEQ ID NOs: 524 to 559.

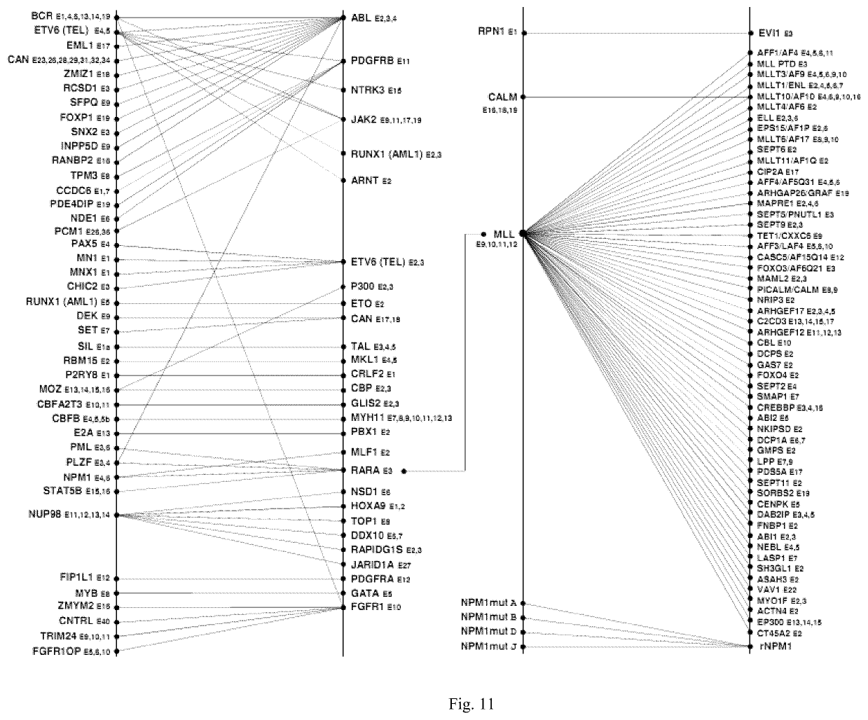

### FIG. 11: Identification of the Fusion Genes in the Case of Leukemias.

The use of probes SEQ ID NOs: 1 to 31, 66 to 219, 438, 467, 534, 559, 616 to 639, 641 to 670, 737 and 759 in a single kit would allow simultaneous searching for more than 110 different gene rearrangements in leukemias.

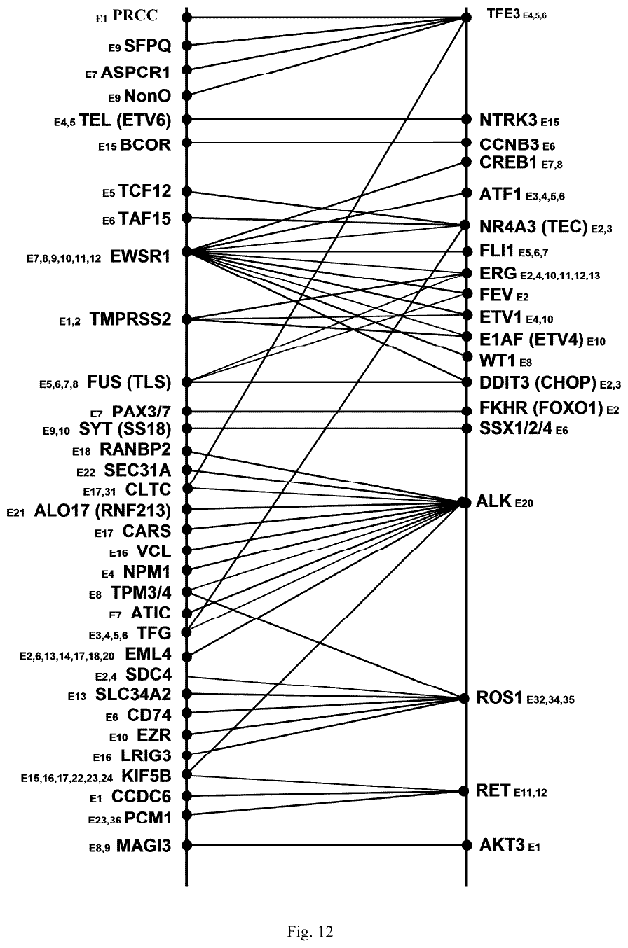

### FIG. 12: Identification of the Fusion Genes in the Case of Sarcomas and Carcinomas.

The fusion transcripts hybridize to the probes SEQ ID NOs: 374 to 405, 524 to 559 and 438 to 480.

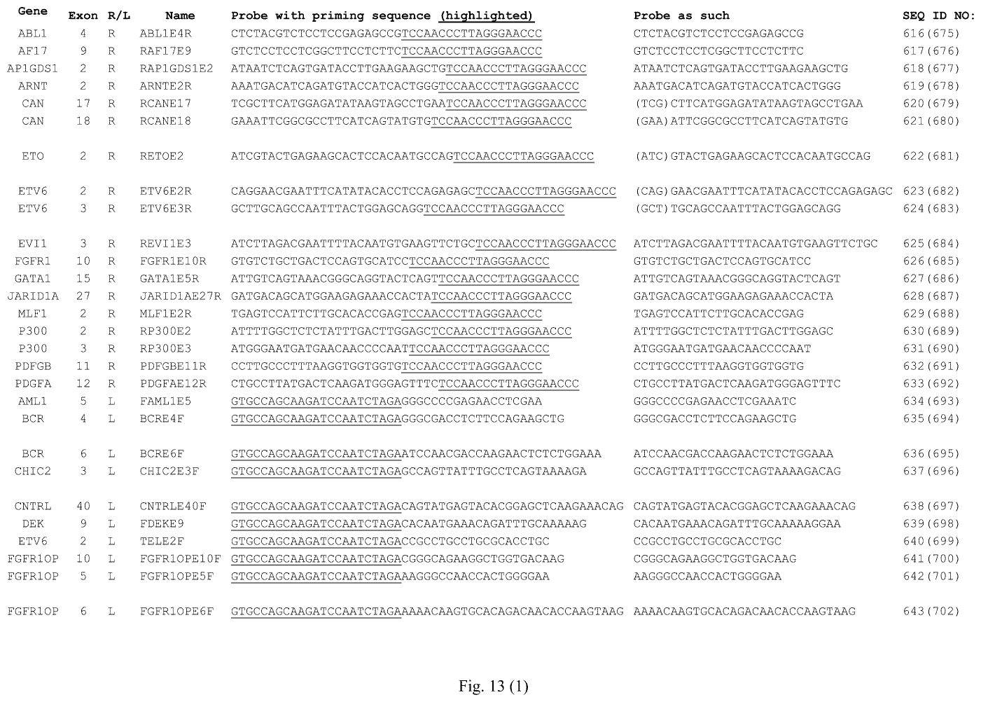

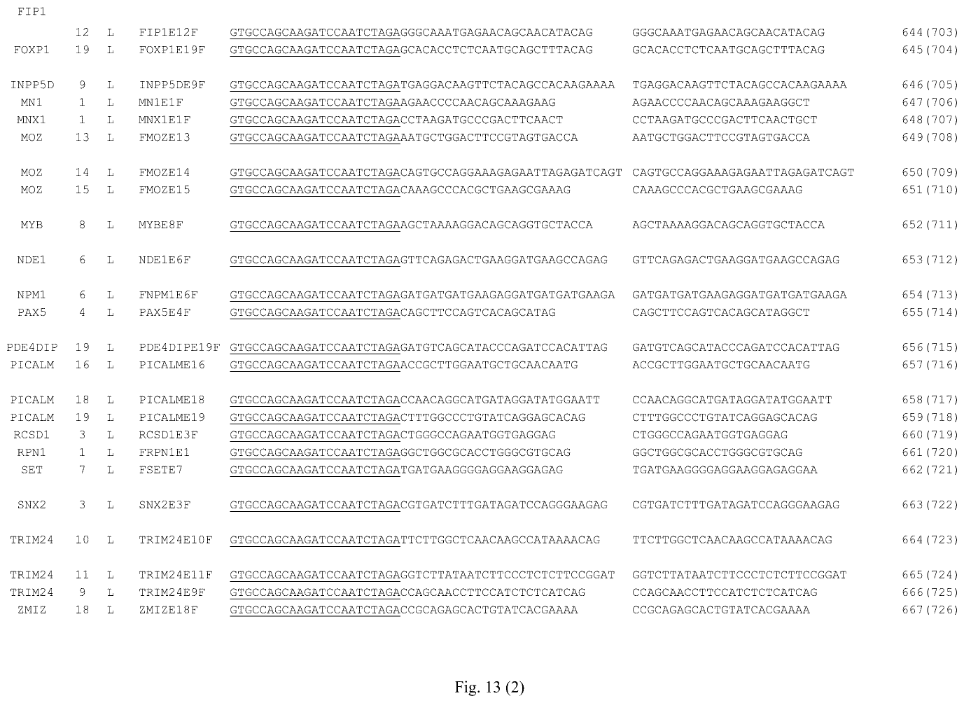

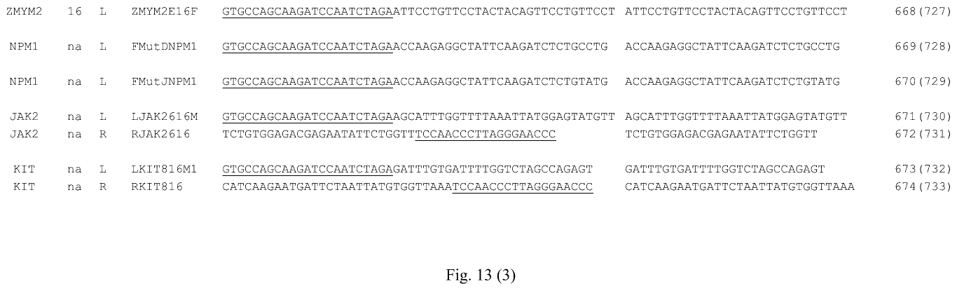

FIG. 13(1)-13(3): Table of Probes SEQ ID NOs: 616-674 (Corresponding to the Probes Fused with the Priming Sequence SEQ ID NOs: 675-733).

The table shows the gene and the exon to which the probe hybridizes. The column “SEQ ID NOs:” gives the sequence number of the probe as such, and in parentheses the sequence number of the probe fused with the priming sequence.

These probes are usable notably for diagnosis of leukemias.

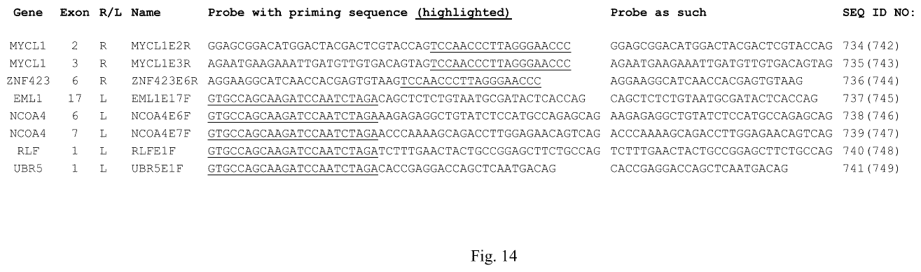

FIG. 14: Table of Probes SEQ ID NOs: 734-741 (Corresponding to the Probes Fused with the Priming Sequence SEQ ID NOs: 742-749).

The table shows the gene and the exon to which the probe hybridizes. The column “SEQ ID NOs:” gives the sequence number of the probe as such, and in parentheses the sequence number of the probe fused with the priming sequence.

These probes are usable notably for diagnosis of carcinomas.

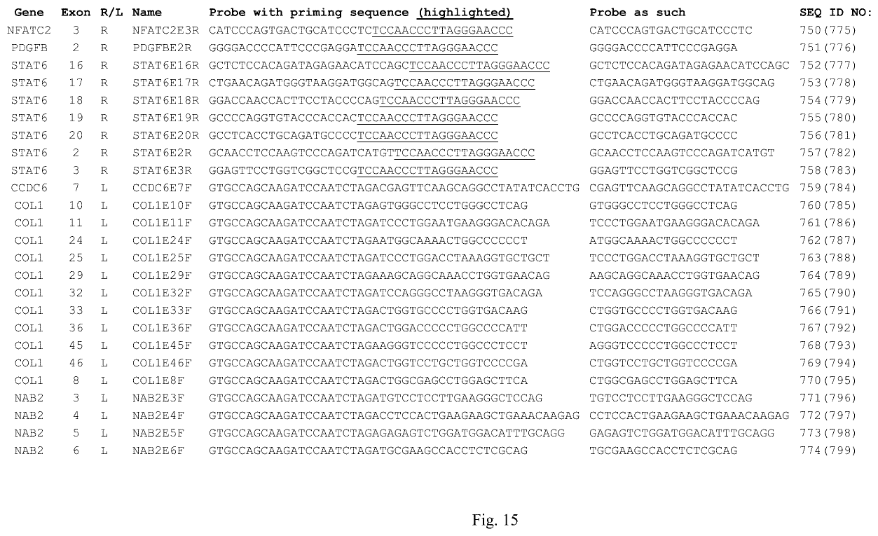

FIG. 15: Table of Probes SEQ ID NOs: 750-774 (Corresponding to the Probes Fused with the Priming Sequence SEQ ID NOs: 775-799).

The table shows the gene and the exon to which the probe hybridizes. The column “SEQ ID NOs:” gives the sequence number of the probe as such, and in parentheses the sequence number of the probe fused with the priming sequence.

These probes are usable notably for diagnosis of carcinomas.

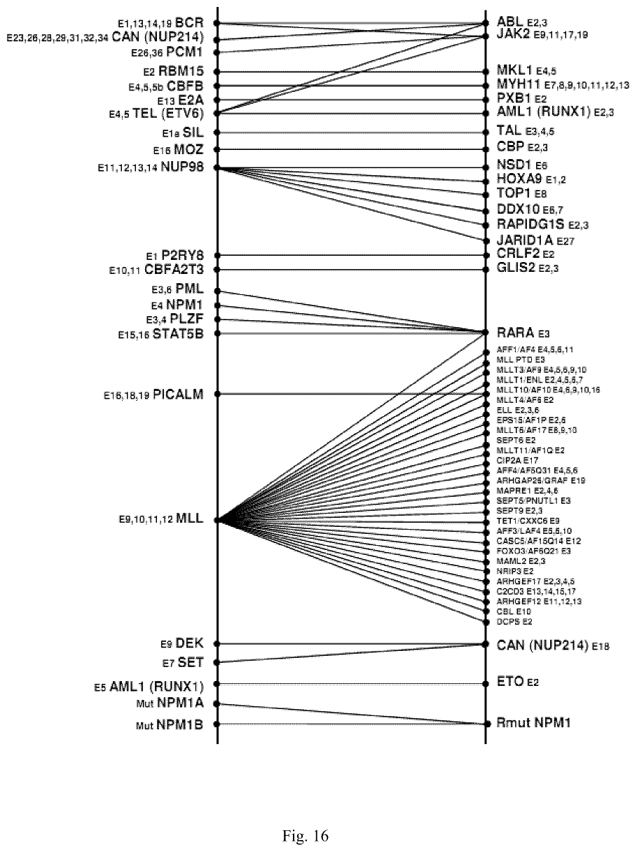

### FIG. 16: Identification of the Fusion Genes Targeted in Example 2.

The fusion transcripts hybridize to the probes SEQ ID NOs: 1 to 31, 66 to 167, 169 to 182, 628, 657 to 659 and 662.

### EXAMPLE 1

### Material and Methods

### Step a): Extraction of RNA:

The cells obtained from blood or from bone marrow or from biopsy fragments are stored at −80° C. in 1 mL of trizol (Life Technologies, Carlsbad, CA, USA).

200 μL of chloroform (Merck, Darmstadt, Germany) is added to each previously thawed sample.

The mixture is homogenized before being incubated for about 5 min in ice and then centrifuged for 15 min at 12000 rpm and 4° C.

500 μL of 100% isopropanol (Sigma Aldrich, St Quentin Fallavier, France) is added to the previously isolated aqueous phase.

The mixture is homogenized before being incubated for about 10 min in ice and centrifuged for 10 min at 12000 rpm and 4° C.

After decanting, the sediments are washed with about 1 mL of 70% ethanol and centrifuged for 5 min at 7500 rpm and 4° C.

After another 2 washing operations the sediments are decanted and dried before being taken up in about 100 μL of water.

The RNAs are then incubated on a water bath at about 55° C. for 5 to 10 min.

The concentration of the RNAs obtained is adjusted to between 50 and 250 ng/μL.

### Step b): Reverse Transcription:

4 μL RNA (200-1000 ng) is incubated with 2.5 μL Buffer 5× (Thermo Fisher Scientific, Waltham, MA, USA), 1 μl DTT 100 mM (Thermo Fisher Scientific, Waltham, MA, USA), 2 μl dNTPs 10 mM (Thermo Fisher Scientific, Waltham, MA, USA) and 2 μl of hexamers at 100 pmol/μl (Thermo Fisher Scientific, Waltham, MA, USA), for 2 minutes at 80° C., then 5 minutes at 37° C., and then the samples are stored at 4° C.

Reverse transcriptase (RT) is then added (1 μL RT (30 u) (M-MLV, Thermo Fisher Scientific, Waltham, MA, USA)), and the resultant mixture is incubated for 15 minutes at 37° C., then 2 minutes at 98° C., and then stored at 4° C.

Step c): Incubation and Hybridization of the cDNA to the Probes (MLPA):

5 μl of the preceding mixture (˜half of the initial reaction mixture) obtained in step b) is incubated with 1.5 μL MLPA Buffer (Old Salsa MLPA Buffer. MRC Holland, Amsterdam, the Netherlands) and 1.5 μL of the mixture of probes described below, for 2 minutes at 95° C., and then at least 1 hour at 60° C.

Mixture of Probes for MLPA (3 fmol/1.5 μl Final):
First step: Taking up each probe with 100 M (H2O)
Second step: Dilution of each probe with 10 μM (H2O)
Third step: Mixture of probes: 2 μl of each probe at 10 μM
Final volume:+volume idem TE20:2 for Mix to final TE10:1
Fourth step: Final dilution: (0.2 μl of the mixture of probes F and R from step 3× in probes)+TE10:1 q.s. 1 ml
   (example: mixture of probes F with 10 different probes: 0.2×10=2 μl mixture F; mixture R with 10 probes: 0.×10=4 μl mixture R; Final Mixture=2 μl mixture F+4 μl mixture R+994 μl TE10:1)

### Step d): Ligation:

Ligase 65 (n tubes+10%) (Salsa Ligase-65. MRC-Holland, Amsterdam, the Netherlands) is prepared with the following mixture:
3 μL Ligase Buffer A (Ligase Buffer A, MRC-Holland, Amsterdam, the Netherlands),
3 μL Ligase Buffer B (Ligase Buffer B, MRC-Holland, Amsterdam, the Netherlands), 25 μL H2O
Vortex
1 μL ligase 65 mix (Salsa Ligase-65. MRC-Holland, Amsterdam, the Netherlands)

### Vortex.

The mixture of probes from step c) is mixed at 54° C. with 32 μL of this ligase mix, then incubated for 15 minutes at 54° C., and 5 minutes at 98° C., and then stored at 4° C.

### Step e): PCR:

PCR (n tubes+10%) is carried out in the following mixture:
20 μl of Eurogentec Red'y′Start Mix (Eurogentec, Angers, France)
1 μl of primer SEQ ID NO: 32 (Biot)
1 μl of primer SEQ ID NO: 33
13 μl H₂O

5 μl of the mixture from step d) is mixed with 35 μl of the above PCR mixture, and is then submitted to the following PCR programme:
94° C. 6 min
94° C. 30 s
35×58° C. 30 s
72°° C. 30 s
72°° C. 4 min
4° C.

### Step f): Pyrosequencing:

Control (optional): 8% acrylamide gel (Acrylamide/Bis-Acrylamide 29:1, 40%, Biosolve B.V., Valkenswaard, the Netherlands)

20 μl of the product amplified by PCR is analyzed by pyrosequencer.

### Results

The results obtained are presented in FIGS. 2A-2F, 3A-3B and 6A for the leukemia cases, and in FIG. 6B for the solid tumors.

At the end of PCR, if the translocation that is being sought is detected, the sample is positive, and the diagnosis of the disease is immediate.

### EXAMPLE 2

In acute leukemia, recurrent chromosome translocations that lead to fusion of two genes are frequent. Certain of these markers have a well established prognostic and therapeutic impact and are systematically monitored at the time of diagnosis by cytogenetics and RT-PCR. However, owing to the limitations of these methods, only a few known rearrangements among all those that exist are systematically assayed. A great many abnormalities that could supply important clinical information are thus ignored, mainly because it is impossible to perform cost-effective, quick and reliable multi-target screening. The assay proposed here is simple and allows reliable detection of dozens of fusion genes in just a few hours.

The assay has been designed for simultaneous detection of more than 50 translocations involving 70 recurrent genes in acute myeloblastic leukemias (AML), acute lymphoblastic leukemias (ALL) and chronic myeloid leukemias (CML).

Samples of cDNA obtained from leukemic cells are first incubated with a mixture of oligonucleotide probes that are complementary to the ends of the exons, at the abnormal junctions on the fusion mRNAs. These probes correspond to the fusion transcripts listed in FIG. 16, and have the sequence SEQ ID NOs: 1 to 31, 66 to 167, 169 to 182, 628, 657 to 659 and 662. For most of the genes, different probes have been designed for detecting different transcripts resulting from alternative genomic recombinations (for example on exons 1, 13, 14 and 19 for the BCR gene, and on exons 2 and 3 for the ABL gene). The mixture thus combines more than 150 probes and is aimed at more than 400 different fusion transcripts. All the probes on the left have a common tail (SA) at their 5′ end, and all the probes on the right have a common tail (SB) at their 3′ end (cf. FIGS. 2A to E). Additional probes have also been included for detecting the most frequent mutations of the NPM1 gene (A, B, D). If a translocation is present in the sample, the two probes hybridize to one another side by side on the fusion cDNA. A DNA ligase is then used for creating a covalent bond between these probes, which allows them to be amplified by PCR with the primers S_{A} and S_{B}. If a PCR product is amplified, the two partners are identified by sequence analysis.

This method was applied to a retrospective series of 430 patients (252 AML and 178 children with ALL). In the ALL-B (147 cases), the 33 rearrangements ETV6-RUNX1, the 6 rearrangements BCR-ABL and the 5 rearrangements TCF3-PBX1, as well as the 6 rearrangements MLL (3 AF4; 1 ENL, 1 AF9 and 1 AFF4) identified at the time of diagnosis by conventional methods, were detected, as well as 5 previously unknown junctions P2RY8-CRLF2.

In the ALL-T (31 cases), 6 known rearrangements (4 SIL-TAL, 2 CALM-AF10) and 5 fusions not detected previously (2 NUP214-ABL, 1 MLL-ENL, 1 ETV6-ABL, and a new junction PLZF-ABL) were detected.

In the AMLs, 86 fusions were detected: 23 PML-RARA, 2 PLZF-RARA, 18 CBFB-MYH11, 12 RUNX1-RUNX1T1, 4 NUP98-NSD1, 2 BCR-ABL, 1 DEK-NUP214, 1 CALM-AF10, 1 MOZ-CBP, 22 rearrangements MLL (13 PTD, 3 AF9, 2 AF6, 1 AF10, 1 ENL, 1 AF1Q and 1 MAPRE) and 44 mutations of NPM1. In particular, 20 translocations of this series, including 14 fusions of the MLL gene and one cryptic cytogenetic abnormality t(8, 21) had not been identified in the diagnosis.

Moreover, all these new abnormalities (in the AMLs and ALLs) could be confirmed by conventional RT-PCR and sequencing, demonstrating the specificity of the method. In the whole cohort of 430 patients, the three methods thus detected 157 fusions. 85 fusions (54.1%) and 112 fusions (71.3%) were detected at the time of diagnosis by cytogenetics or by RT-PCR respectively, and 152 fusions (96.8%) were detected by the present method.

In conclusion, the method according to the invention is a simple multiplex assay that can reveal a very large number of recurrent gene fusions in leukemia. Its short turnaround (up to 40 patients can be tested in parallel and the results can be obtained in less than a day) and its low cost (just PCR apparatus, a pyrosequencer and basic reagents for molecular biology are required) make it particularly suitable for everyday practice. Its ability to detect a great many abnormalities that are hardly ever tested in everyday practice could supply many diagnoses and prognoses, and allow stratification of patients in prospective clinical trials.

1. A method for detecting a chromosome rearrangement in a subject comprising at least the following steps:
a) extraction of RNA from a biological sample from the subject;
b) conversion of the RNA extracted in a) to cDNA by reverse transcription;
c) incubation of the cDNA obtained in b) with at least one pair of probes selected from:
probes SEQ ID NO: 1 to 25, 30, 31 and 113 to 120,
probes SEQ ID NO: 374 to 405, and
probes SEQ ID NO: 524 to 559,
each of the probes being fused, at one end at least, with a priming sequence,
d) addition of a DNA ligase to the mixture obtained in c), in order to establish a covalent bond between two contiguous probes;
e) PCR amplification of the covalently bound contiguous probes obtained in d),
f) detecting chromosome rearrangement in the subject based on PCR amplicons being produced in step e) following hybridization to said two contiguous probes.

2. The method as claimed in claim 1, in which the incubation of step c) is carried out with at least the probes SEQ ID NO: 1 to 25, 30, 31 and 113 to 120, and/or with at least the probes SEQ ID NO: 374 to 405, and/or with at least the probes SEQ ID NO: 524 to 559, each of the probes being fused, at one end at least, with a priming sequence.

3. The method as claimed in claim 1, wherein:
PCR amplification with the probes SEQ ID NOs: 1 to 25, 30, 31 and 113 to 120 is representative of a subject suffering from leukemia connected with a chromosome rearrangement on gene ABL, BCR, PML, RARA, CBFB, MYH11, E2A (TCF3), PBX1, TEL (ETV6), AML1 (RUNX1), ETO, MLL, and/or AFF1/AF4, and/or
PCR amplification with the probes SEQ ID NOs: 374 to 405 is representative of a subject suffering from sarcoma connected with a chromosome rearrangement on gene EWSR1, FLI1, ERG, FEV, ETV1, E1AF (ETV4), WT1, FUS (TLS), PAX3/7, DDIT3 (CHOP), FKHR (FOXO1), SSX, and/or SYT (SS18), and/or
PCR amplification with the probes SEQ ID NOs: 524 to 559 is representative of a subject suffering from carcinoma connected with a chromosome rearrangement on gene TMPRSS2, VCL, ERG, ALK, EML4, TPM3/4, SDC4, SLC34A2, ROS1, CD74, EZR, LRIG3, KIF5B, CCDC6, and/or RET.

4. The method as claimed in claim 1, wherein the priming sequence is selected from the sequences of the group consisting of SEQ ID NO: 33 and SEQ ID NO: 34.

5. The method as claimed in claim 1, wherein said biological sample is selected from whole blood, bone marrow and a biopsy from said subject.

6. The method as claimed in claim 1, further comprising using the probes SEQ ID NO: 26 to 29, 66 to 112 and 121 to 219 and/or the probes SEQ ID NO: 616 to 674 in the incubation of step c), each of the probes being fused, at one end at least, with a priming sequence.

7. The method as claimed in claim 1, further comprising using the probes SEQ ID NO: 750 to 774 in the incubation of step c), each of the probes being fused, at one end at least, with a priming sequence.

8. The method as claimed in claim 1, further comprising using the probes SEQ ID NO: 734 to 741 in the incubation of step c), each of the probes being fused, at one end at least, with a priming sequence.

9. The method as claimed in claim 1, wherein the incubation of step c) is carried out with at least the probes SEQ ID NO: 1 to 25, 30, 31 and 113 to 120, at least the probes SEQ ID NO: 374 to 405, and with at least the probes SEQ ID NO: 524 to 559, each of the probes being fused, at one end at least, with a priming sequence.

10. The method as claimed in claim 9, wherein the step f) of analysis of the results of the PCR is carried out by pyrosequencing.

11. The method as claimed in claim 1, wherein the PCR amplification in step e) is carried out using the primers SEQ ID NO: 32 and 33.

12. The method as claimed in claim 1, further comprising using the probes SEQ ID NO: 35 to 59, 64, 65 and 267 to 274 in the incubation of step c).

13. The method as claimed in claim 12, further comprising using the probes SEQ ID NO: 60 to 63, 220 to 266 and 275 to 373, and/or the probes SEQ ID NO: 675 to 733 in the incubation of step c).

14. The method as claimed in claim 1, further comprising using the probes SEQ ID NO: 406 to 437 in the incubation of step c).

15. The method as claimed in claim 14, further comprising using the probes SEQ ID NO: 481 to 523 and/or the probes SEQ ID NO: 775 to 799 in the incubation of step c).

16. The method as claimed in claim 1, further comprising using the probes SEQ ID NO: 560 to 595 in the incubation of step c).

17. The method as claimed in claim 16, further comprising using the probes SEQ ID NO: 481 to 523 and/or the probes SEQ ID NO: 742 to 749 in the incubation of step c).

18. A kit suitable for PCR amplification comprising at least the probes SEQ ID NO: 1 to 25, 30, 31 and 113 to 120, and/or the probes SEQ ID NO: 374 to 405, and/or the probes SEQ ID NO: 524 to 559, each of the probes being fused, at one end at least, with a priming sequence.

19. The kit of claim 18 further comprising the probes SEQ ID NO: 26 to 29, 66 to 112 and 121 to 219, and/or the probes SEQ ID NO: 616 to 674, and/or the probes SEQ ID NO: 438 to 480, and/or the probes SEQ ID NO: 750 to 774, and/or the probes SEQ ID NO: 734 to 741, each of the probes being fused, at one end at least, with a priming sequence.

20. A kit suitable for PCR amplification comprising at least the probes SEQ ID NO: 35 to 59, 64, 65 and 267 to 274, and/or the probes SEQ ID NO: 406 to 437, and/or the probes SEQ ID NO: 560 to 595.

21. The kit of claim 20 further comprising the probes SEQ ID NO: 60 to 63, 220 to 266 and 275 to 373, and/or the probes SEQ ID NO: 675 to 733, and/or the probes SEQ ID NO: 775 to 799, and/or the probes SEQ ID NO: 742 to 749.


SEQUENCE LISTING (799 sequences)

SEQ ID NO: 1 - DNA, 22 bp, synthetic construct
  misc_feature 1..22: note = primer
  source 1..22: mol_type = other DNA
aagcccttcagcggccagtagc

SEQ ID NO: 2 - DNA, 23 bp, synthetic construct
  misc_feature 1..23: note = primer
  source 1..23: mol_type = other DNA
gtgaaaagctccgggtcttaggc

SEQ ID NO: 3 - DNA, 22 bp, synthetic construct
  misc_feature 1..22: note = primer
  source 1..22: mol_type = other DNA
ggcgccttccatggagacgcag

SEQ ID NO: 4 - DNA, 25 bp, synthetic construct
  misc_feature 1..25: note = primer
  source 1..25: mol_type = other DNA
attccgctgaccatcaataaggaag

SEQ ID NO: 5 - DNA, 26 bp, synthetic construct
  misc_feature 1..26: note = primer
  source 1..26: mol_type = other DNA
agccactggatttaagcagagttcaa

SEQ ID NO: 6 - DNA, 23 bp, synthetic construct
  misc_feature 1..23: note = primer
  source 1..23: mol_type = other DNA
actgaaggcagccttcgacgtca

SEQ ID NO: 7 - DNA, 24 bp, synthetic construct
  misc_feature 1..24: note = primer
  source 1..24: mol_type = other DNA
gctcttgcatcacccaggggaaag

SEQ ID NO: 8 - DNA, 20 bp, synthetic construct
  misc_feature 1..20: note = primer
  source 1..20: mol_type = other DNA
cagtggcgccggggaggcag

SEQ ID NO: 9 - DNA, 25 bp, synthetic construct
  misc_feature 1..25: note = primer
  source 1..25: mol_type = other DNA
ccattgagacccagagcagcagttc

SEQ ID NO: 10 - DNA, 24 bp, synthetic construct
  misc_feature 1..24: note = primer
  source 1..24: mol_type = other DNA
gagtttgatgaggagcgagcccag

SEQ ID NO: 11 - DNA, 25 bp, synthetic construct
  misc_feature 1..25: note = primer
  source 1..25: mol_type = other DNA
caggtctcatcgggaggaaatggag

SEQ ID NO: 12 - DNA, 26 bp, synthetic construct
  misc_feature 1..26: note = primer
  source 1..26: mol_type = other DNA
agtttcacagctgctggcagtaactg

SEQ ID NO: 13 - DNA, 27 bp, synthetic construct
  misc_feature 1..27: note = primer
  source 1..27: mol_type = other DNA
gccaaggcgaacctagacaagaataag

SEQ ID NO: 14 - DNA, 26 bp, synthetic construct
  misc_feature 1..26: note = primer
  source 1..26: mol_type = other DNA
aatgaagttgagagcgtcacagggat

SEQ ID NO: 15 - DNA, 25 bp, synthetic construct
  misc_feature 1..25: note = primer
  source 1..25: mol_type = other DNA
gagctgcttcaagaagaaacccggc

SEQ ID NO: 16 - DNA, 25 bp, synthetic construct
  misc_feature 1..25: note = primer
  source 1..25: mol_type = other DNA
ctctccgactcgaagaagaagctgc

SEQ ID NO: 17 - DNA, 29 bp, synthetic construct
  misc_feature 1..29: note = primer
  source 1..29: mol_type = other DNA
ttgttagccgaggagaaaaacatctcttc

SEQ ID NO: 18 - DNA, 25 bp, synthetic construct
  misc_feature 1..25: note = primer
  source 1..25: mol_type = other DNA
gtccatgagctggagaagtccaagc

SEQ ID NO: 19 - DNA, 28 bp, synthetic construct
  misc_feature 1..28: note = primer
  source 1..28: mol_type = other DNA
cttcacgagtatgagacggaactggaag

SEQ ID NO: 20 - DNA, 25 bp, synthetic construct
  misc_feature 1..25: note = primer
  source 1..25: mol_type = other DNA
tctcggcctcccgactcctacagtg

SEQ ID NO: 21 - DNA, 26 bp, synthetic construct
  misc_feature 1..26: note = primer
  source 1..26: mol_type = other DNA
ttttgagtatccgaggagcccaggag

SEQ ID NO: 22 - DNA, 29 bp, synthetic construct
  misc_feature 1..29: note = primer
  source 1..29: mol_type = other DNA
ggtcatactgcatcagaaccatgaagaag

SEQ ID NO: 23 - DNA, 24 bp, synthetic construct
  misc_feature 1..24: note = primer
  source 1..24: mol_type = other DNA
ccatgcccattgggagaatagcag

SEQ ID NO: 24 - DNA, 30 bp, synthetic construct
  misc_feature 1..30: note = primer
  source 1..30: mol_type = other DNA
aatgcatacttggaatgaatccttctagag

SEQ ID NO: 25 - DNA, 23 bp, synthetic construct
  misc_feature 1..23: note = primer
  source 1..23: mol_type = other DNA
atgccagcacgagccgccgcttc

SEQ ID NO: 26 - DNA, 23 bp, synthetic construct
  misc_feature 1..23: note = primer
  source 1..23: mol_type = other DNA
acctcagctccgcggaagttgcg

SEQ ID NO: 27 - DNA, 22 bp, synthetic construct
  misc_feature 1..22: note = primer
  source 1..22: mol_type = other DNA
atcgcccaggaccacaccgcag

SEQ ID NO: 28 - DNA, 22 bp, synthetic construct
  misc_feature 1..22: note = primer
  source 1..22: mol_type = other DNA
gatgaccgagcggccgccgagc

SEQ ID NO: 29 - DNA, 22 bp, synthetic construct
  misc_feature 1..22: note = primer
  source 1..22: mol_type = other DNA
gtccccacaccaaagttgtgcg

SEQ ID NO: 30 - DNA, 24 bp, synthetic construct
  misc_feature 1..24: note = primer
  source 1..24: mol_type = other DNA
tggatgggccccgagaacctcgaa

SEQ ID NO: 31 - DNA, 27 bp, synthetic construct
  misc_feature 1..27: note = primer
  source 1..27: mol_type = other DNA
atcgtactgagaagcactccacaatgc

SEQ ID NO: 32 - DNA, 19 bp, synthetic construct
  misc_feature 1..19: note = primer
  source 1..19: mol_type = other DNA
gggttccctaagggttgga

SEQ ID NO: 33 - DNA, 23 bp, synthetic construct
  misc_feature 1..23: note = primer
  source 1..23: mol_type = other DNA
gtgccagcaagatccaatctaga

SEQ ID NO: 34 - DNA, 19 bp, synthetic construct
  misc_feature 1..19: note = primer
  source 1..19: mol_type = other DNA
tccaacccttagggaaccc

SEQ ID NO: 35 - DNA, 41 bp, synthetic construct
  misc_feature 1..41: note = primer
  source 1..41: mol_type = other DNA
aagcccttcagcggccagtagctccaacccttagggaaccc

SEQ ID NO: 36 - DNA, 42 bp, synthetic construct
  misc_feature 1..42: note = primer
  source 1..42: mol_type = other DNA
gtgaaaagctccgggtcttaggctccaacccttagggaaccc

SEQ ID NO: 37 - DNA, 45 bp, synthetic construct
  misc_feature 1..45: note = primer
  source 1..45: mol_type = other DNA
gtgccagcaagatccaatctagaggcgccttccatggagacgcag

SEQ ID NO: 38 - DNA, 48 bp, synthetic construct
  misc_feature 1..48: note = primer
  source 1..48: mol_type = other DNA
gtgccagcaagatccaatctagaattccgctgaccatcaataaggaag

SEQ ID NO: 39 - DNA, 49 bp, synthetic construct
  misc_feature 1..49: note = primer
  source 1..49: mol_type = other DNA
gtgccagcaagatccaatctagaagccactggatttaagcagagttcaa

SEQ ID NO: 40 - DNA, 46 bp, synthetic construct
  misc_feature 1..46: note = primer
  source 1..46: mol_type = other DNA
gtgccagcaagatccaatctagaactgaaggcagccttcgacgtca

SEQ ID NO: 41 - DNA, 47 bp, synthetic construct
  misc_feature 1..47: note = primer
  source 1..47: mol_type = other DNA
gtgccagcaagatccaatctagagctcttgcatcacccaggggaaag

SEQ ID NO: 42 - DNA, 43 bp, synthetic construct
  misc_feature 1..43: note = primer
  source 1..43: mol_type = other DNA
gtgccagcaagatccaatctagacagtggcgccggggaggcag

SEQ ID NO: 43 - DNA, 44 bp, synthetic construct
  misc_feature 1..44: note = primer
  source 1..44: mol_type = other DNA
ccattgagacccagagcagcagttctccaacccttagggaaccc

SEQ ID NO: 44 - DNA, 47 bp, synthetic construct
  misc_feature 1..47: note = primer
  source 1..47: mol_type = other DNA
gtgccagcaagatccaatctagagagtttgatgaggagcgagcccag

SEQ ID NO: 45 - DNA, 48 bp, synthetic construct
  misc_feature 1..48: note = primer
  source 1..48: mol_type = other DNA
gtgccagcaagatccaatctagacaggtctcatcgggaggaaatggag

SEQ ID NO: 46 - DNA, 49 bp, synthetic construct
  misc_feature 1..49: note = primer
  source 1..49: mol_type = other DNA
gtgccagcaagatccaatctagaagtttcacagctgctggcagtaactg

SEQ ID NO: 47 - DNA, 46 bp, synthetic construct
  misc_feature 1..46: note = primer
  source 1..46: mol_type = other DNA
gccaaggcgaacctagacaagaataagtccaacccttagggaaccc

SEQ ID NO: 48 - DNA, 45 bp, synthetic construct
  misc_feature 1..45: note = primer
  source 1..45: mol_type = other DNA
aatgaagttgagagcgtcacagggattccaacccttagggaaccc

SEQ ID NO: 49 - DNA, 44 bp, synthetic construct
  misc_feature 1..44: note = primer
  source 1..44: mol_type = other DNA
gagctgcttcaagaagaaacccggctccaacccttagggaaccc

SEQ ID NO: 50 - DNA, 44 bp, synthetic construct
  misc_feature 1..44: note = primer
  source 1..44: mol_type = other DNA
ctctccgactcgaagaagaagctgctccaacccttagggaaccc

SEQ ID NO: 51 - DNA, 48 bp, synthetic construct
  misc_feature 1..48: note = primer
  source 1..48: mol_type = other DNA
ttgttagccgaggagaaaaacatctcttctccaacccttagggaaccc

SEQ ID NO: 52 - DNA, 44 bp, synthetic construct
  misc_feature 1..44: note = primer
  source 1..44: mol_type = other DNA
gtccatgagctggagaagtccaagctccaacccttagggaaccc

SEQ ID NO: 53 - DNA, 47 bp, synthetic construct
  misc_feature 1..47: note = primer
  source 1..47: mol_type = other DNA
cttcacgagtatgagacggaactggaagtccaacccttagggaaccc

SEQ ID NO: 54 - DNA, 48 bp, synthetic construct
  misc_feature 1..48: note = primer
  source 1..48: mol_type = other DNA
gtgccagcaagatccaatctagatctcggcctcccgactcctacagtg

SEQ ID NO: 55 - DNA, 45 bp, synthetic construct
  misc_feature 1..45: note = primer
  source 1..45: mol_type = other DNA
ttttgagtatccgaggagcccaggagtccaacccttagggaaccc

SEQ ID NO: 56 - DNA, 52 bp, synthetic construct
  misc_feature 1..52: note = primer
  source 1..52: mol_type = other DNA
gtgccagcaagatccaatctagaggtcatactgcatcagaaccatgaagaag

SEQ ID NO: 57 - DNA, 47 bp, synthetic construct
  misc_feature 1..47: note = primer
  source 1..47: mol_type = other DNA
gtgccagcaagatccaatctagaccatgcccattgggagaatagcag

SEQ ID NO: 58 - DNA, 49 bp, synthetic construct
  misc_feature 1..49: note = primer
  source 1..49: mol_type = other DNA
aatgcatacttggaatgaatccttctagagtccaacccttagggaaccc

SEQ ID NO: 59 - DNA, 42 bp, synthetic construct
  misc_feature 1..42: note = primer
  source 1..42: mol_type = other DNA
atgccagcacgagccgccgcttctccaacccttagggaaccc

SEQ ID NO: 60 - DNA, 46 bp, synthetic construct
  misc_feature 1..46: note = primer
  source 1..46: mol_type = other DNA
gtgccagcaagatccaatctagaacctcagctccgcggaagttgcg

SEQ ID NO: 61 - DNA, 41 bp, synthetic construct
  misc_feature 1..41: note = primer
  source 1..41: mol_type = other DNA
atcgcccaggaccacaccgcagtccaacccttagggaaccc

SEQ ID NO: 62 - DNA, 41 bp, synthetic construct
  misc_feature 1..41: note = primer
  source 1..41: mol_type = other DNA
gatgaccgagcggccgccgagctccaacccttagggaaccc

SEQ ID NO: 63 - DNA, 41 bp, synthetic construct
  misc_feature 1..41: note = primer
  source 1..41: mol_type = other DNA
gtccccacaccaaagttgtgcgtccaacccttagggaaccc

SEQ ID NO: 64 - DNA, 47 bp, synthetic construct
  misc_feature 1..47: note = primer
  source 1..47: mol_type = other DNA
gtgccagcaagatccaatctagatggatgggccccgagaacctcgaa

SEQ ID NO: 65 - DNA, 46 bp, synthetic construct
  misc_feature 1..46: note = primer
  source 1..46: mol_type = other DNA
atcgtactgagaagcactccacaatgctccaacccttagggaaccc

SEQ ID NO: 66 - DNA, 23 bp, synthetic construct
  misc_feature 1..23: note = primer
  source 1..23: mol_type = other DNA
gcagatggccagtcaggcaccag

SEQ ID NO: 67 - DNA, 29 bp, synthetic construct
  misc_feature 1..29: note = primer
  source 1..29: mol_type = other DNA
tcagttatcatctggtgacaaagcttcag

SEQ ID NO: 68 - DNA, 27 bp, synthetic construct
  misc_feature 1..27: note = primer
  source 1..27: mol_type = other DNA
cgtcttctaatttcactgctgcacaag

SEQ ID NO: 69 - DNA, 24 bp, synthetic construct
  misc_feature 1..24: note = primer
  source 1..24: mol_type = other DNA
ggttcagcttttgccaagcttcag

SEQ ID NO: 70 - DNA, 25 bp, synthetic construct
  misc_feature 1..25: note = primer
  source 1..25: mol_type = other DNA
ggctttggatccacagctacctcaa

SEQ ID NO: 71 - DNA, 26 bp, synthetic construct
  misc_feature 1..26: note = primer
  source 1..26: mol_type = other DNA
tgggttttcctctccaaacaaaacag

SEQ ID NO: 72 - DNA, 23 bp, synthetic construct
  misc_feature 1..23: note = primer
  source 1..23: mol_type = other DNA
tggttttggatcaggcacaggag

SEQ ID NO: 73 - DNA, 30 bp, synthetic construct
  misc_feature 1..30: note = primer
  source 1..30: mol_type = other DNA
attatgaactattaacagaaaatgacatgt

SEQ ID NO: 74 - DNA, 28 bp, synthetic construct
  misc_feature 1..28: note = primer
  source 1..28: mol_type = other DNA
ttcttcaggagagaataccatgggtacc

SEQ ID NO: 75 - DNA, 30 bp, synthetic construct
  misc_feature 1..30: note = primer
  source 1..30: mol_type = other DNA
gaaattgaacttagctcattaagggaagct

SEQ ID NO: 76 - DNA, 30 bp, synthetic construct
  misc_feature 1..30: note = primer
  source 1..30: mol_type = other DNA
cgagaaaatgtcattgaatataaacactgt

SEQ ID NO: 77 - DNA, 30 bp, synthetic construct
  misc_feature 1..30: note = primer
  source 1..30: mol_type = other DNA
cctagtgagagccttgctactactgatgat

SEQ ID NO: 78 - DNA, 30 bp, synthetic construct
  misc_feature 1..30: note = primer
  source 1..30: mol_type = other DNA
actgaatctccagtgttagtgaatgactat

SEQ ID NO: 79 - DNA, 30 bp, synthetic construct
  misc_feature 1..30: note = primer
  source 1..30: mol_type = other DNA
cagtgcatattagtggacagcacttagtag

SEQ ID NO: 80 - DNA, 28 bp, synthetic construct
  misc_feature 1..28: note = primer
  source 1..28: mol_type = other DNA
ctgagaatgcacttactggctcattcag

SEQ ID NO: 81 - DNA, 26 bp, synthetic construct
  misc_feature 1..26: note = primer
  source 1..26: mol_type = other DNA
ggagacacacaggcagacccatactg

SEQ ID NO: 82 - DNA, 28 bp, synthetic construct
  misc_feature 1..28: note = primer
  source 1..28: mol_type = other DNA
atcaccattgcttggaagtttgattctc

SEQ ID NO: 83 - DNA, 26 bp, synthetic construct
  misc_feature 1..26: note = primer
  source 1..26: mol_type = other DNA
accagttccctgcgagtctgctactg

SEQ ID NO: 84 - DNA, 27 bp, synthetic construct
  misc_feature 1..27: note = primer
  source 1..27: mol_type = other DNA
attacctggtcatgatcattgtccgtg

SEQ ID NO: 85 - DNA, 26 bp, synthetic construct
  misc_feature 1..26: note = primer
  source 1..26: mol_type = other DNA
tgctacagttgaaactccagcagcgc

SEQ ID NO: 86 - DNA, 26 bp, synthetic construct
  misc_feature 1..26: note = primer
  source 1..26: mol_type = other DNA
ctttgaaaagtccagccgcatttcat

SEQ ID NO: 87 - DNA, 30 bp, synthetic construct
  misc_feature 1..30: note = primer
  source 1..30: mol_type = other DNA
gaagtcacaatgaaacagatttgcaaaaag

SEQ ID NO: 88 - DNA, 25 bp, synthetic construct
  misc_feature 1..25: note = primer
  source 1..25: mol_type = other DNA
gaaattcggcgccttcatcagtatg

SEQ ID NO: 89 - DNA, 30 bp, synthetic construct
  misc_feature 1..30: note = primer
  source 1..30: mol_type = other DNA
aagaagatgaagagtcagatgatgctgatg

SEQ ID NO: 90 - DNA, 30 bp, synthetic construct
  misc_feature 1..30: note = primer
  source 1..30: mol_type = other DNA
attttggatcattgtttgacttggaaaatg

SEQ ID NO: 91 - DNA, 26 bp, synthetic construct
  misc_feature 1..26: note = primer
  source 1..26: mol_type = other DNA
atgggaataactgggaacacaagtcc

SEQ ID NO: 92 - DNA, 23 bp, synthetic construct
  misc_feature 1..23: note = primer
  source 1..23: mol_type = other DNA
gcttggtgcaggatttggaacag

SEQ ID NO: 93 - DNA, 22 bp, synthetic construct
  misc_feature 1..22: note = primer
  source 1..22: mol_type = other DNA
ggagccccccaggccccagtag

SEQ ID NO: 94 - DNA, 28 bp, synthetic construct
  misc_feature 1..28: note = primer
  source 1..28: mol_type = other DNA
gatgtcagaccctaagaagaaggaagag

SEQ ID NO: 95 - DNA, 24 bp, synthetic construct
  misc_feature 1..24: note = primer
  source 1..24: mol_type = other DNA
agccaatggagcattcatgcccaa

SEQ ID NO: 96 - DNA, 23 bp, synthetic construct
  misc_feature 1..23: note = primer
  source 1..23: mol_type = other DNA
ctgtgcggtcagagaagaaacgc

SEQ ID NO: 97 - DNA, 27 bp, synthetic construct
  misc_feature 1..27: note = primer
  source 1..27: mol_type = other DNA
ttgatagagaaaaacaacccagcgaag

SEQ ID NO: 98 - DNA, 24 bp, synthetic construct
  misc_feature 1..24: note = primer
  source 1..24: mol_type = other DNA
ataacccagcagccaactggcttc

SEQ ID NO: 99 - DNA, 27 bp, synthetic construct
  misc_feature 1..27: note = primer
  source 1..27: mol_type = other DNA
gatggtaaattgaaaaaacccaagaat

SEQ ID NO: 100 - DNA, 27 bp, synthetic construct
  misc_feature 1..27: note = primer
  source 1..27: mol_type = other DNA
ttcttgatgaagcagatagaatcttgg

SEQ ID NO: 101 - DNA, 23 bp, synthetic construct
  misc_feature 1..23: note = primer
  source 1..23: mol_type = other DNA
cacccctgccactttggaacaga

SEQ ID NO: 102 - DNA, 29 bp, synthetic construct
  misc_feature 1..29: note = primer
  source 1..29: mol_type = other DNA
ataatctcagtgataccttgaagaagctg

SEQ ID NO: 103 - DNA, 28 bp, synthetic construct
  misc_feature 1..28: note = primer
  source 1..28: mol_type = other DNA
atacggaaacaagtgaaaaaatccaagc

SEQ ID NO: 104 - DNA, 29 bp, synthetic construct
  misc_feature 1..29: note = primer
  source 1..29: mol_type = other DNA
accaagaggctattcaagatctctgtctg

SEQ ID NO: 105 - DNA, 29 bp, synthetic construct
  misc_feature 1..29: note = primer
  source 1..29: mol_type = other DNA
accaagaggctattcaagatctctgcatg

SEQ ID NO: 106 - DNA, 30 bp, synthetic construct
  misc_feature 1..30: note = primer
  source 1..30: mol_type = other DNA
gcagtggaggaagtctctttaagaaaatag

SEQ ID NO: 107 - DNA, 23 bp, synthetic construct
  misc_feature 1..23: note = primer
  source 1..23: mol_type = other DNA
cttctgccgctgcttctgcacag

SEQ ID NO: 108 - DNA, 23 bp, synthetic construct
  misc_feature 1..23: note = primer
  source 1..23: mol_type = other DNA
gcatggggcggctggttctgctg

SEQ ID NO: 109 - DNA, 24 bp, synthetic construct
  misc_feature 1..24: note = primer
  source 1..24: mol_type = other DNA
ctgaggacatctggaggaaggctg

SEQ ID NO: 110 - DNA, 21 bp, synthetic construct
  misc_feature 1..21: note = primer
  source 1..21: mol_type = other DNA
cagcaggaggactccagcgag

SEQ ID NO: 111 - DNA, 24 bp, synthetic construct
  misc_feature 1..24: note = primer
  source 1..24: mol_type = other DNA
gcttcctgctgaactccaagttcc

SEQ ID NO: 112 - DNA, 23 bp, synthetic construct
  misc_feature 1..23: note = primer
  source 1..23: mol_type = other DNA
gacttccagccactgcgctattt

SEQ ID NO: 113 - DNA, 30 bp, synthetic construct
  misc_feature 1..30: note = primer
  source 1..30: mol_type = other DNA
atccctgtaaaacaaaaaccaaaagaaaag

SEQ ID NO: 114 - DNA, 28 bp, synthetic construct
  misc_feature 1..28: note = primer
  source 1..28: mol_type = other DNA
agtccacaggatcagagtggactttaag

SEQ ID NO: 115 - DNA, 27 bp, synthetic construct
  misc_feature 1..27: note = primer
  source 1..27: mol_type = other DNA
ctctgtgccagtagtgggcatgtagag

SEQ ID NO: 116 - DNA, 26 bp, synthetic construct
  misc_feature 1..26: note = primer
  source 1..26: mol_type = other DNA
gtggaaggcaacatcaggctacaaag

SEQ ID NO: 117 - DNA, 26 bp, synthetic construct
  misc_feature 1..26: note = primer
  source 1..26: mol_type = other DNA
cagacctactccaatgaagtccattg

SEQ ID NO: 118 - DNA, 23 bp, synthetic construct
  misc_feature 1..23: note = primer
  source 1..23: mol_type = other DNA
gaaatgacccattcatggccgcc

SEQ ID NO: 119 - DNA, 26 bp, synthetic construct
  misc_feature 1..26: note = primer
  source 1..26: mol_type = other DNA
gactctcagcatgtcagttctgtaac

SEQ ID NO: 120 - DNA, 26 bp, synthetic construct
  misc_feature 1..26: note = primer
  source 1..26: mol_type = other DNA
cctgagcctccaacaacaaacaaatg

SEQ ID NO: 121 - DNA, 27 bp, synthetic construct
  misc_feature 1..27: note = primer
  source 1..27: mol_type = other DNA
gatgagcaattcttaggttttggctca

SEQ ID NO: 122 - DNA, 25 bp, synthetic construct
  misc_feature 1..25: note = primer
  source 1..25: mol_type = other DNA
gaagaacctaggaaagtccgctttg

SEQ ID NO: 123 - DNA, 27 bp, synthetic construct
  misc_feature 1..27: note = primer
  source 1..27: mol_type = other DNA
gaccctaataggagtattcataccagc

SEQ ID NO: 124 - DNA, 23 bp, synthetic construct
  misc_feature 1..23: note = primer
  source 1..23: mol_type = other DNA
tctgaacaacccagtcctgccag

SEQ ID NO: 125 - DNA, 31 bp, synthetic construct
  misc_feature 1..31: note = primer
  source 1..31: mol_type = other DNA
attcttgaagtgaaaagtccaataaagcaaa

SEQ ID NO: 126 - DNA, 25 bp, synthetic construct
  misc_feature 1..25: note = primer
  source 1..25: mol_type = other DNA
gcatacctagatgaactggtagagc

SEQ ID NO: 127 - DNA, 23 bp, synthetic construct
  misc_feature 1..23: note = primer
  source 1..23: mol_type = other DNA
tgcaccgtccaggtgaggttaga

SEQ ID NO: 128 - DNA, 23 bp, synthetic construct
  misc_feature 1..23: note = primer
  source 1..23: mol_type = other DNA
gaggagccgaggaaggtctgctt

SEQ ID NO: 129 - DNA, 23 bp, synthetic construct
  misc_feature 1..23: note = primer
  source 1..23: mol_type = other DNA
gtgatggtaatgcccgaaggagc

SEQ ID NO: 130 - DNA, 23 bp, synthetic construct
  misc_feature 1..23: note = primer
  source 1..23: mol_type = other DNA
gacgccaacaaggagagcagcaa

SEQ ID NO: 131 - DNA, 23 bp, synthetic construct
  misc_feature 1..23: note = primer
  source 1..23: mol_type = other DNA
tctgcccagtcaagcccgtccaa

SEQ ID NO: 132 - DNA, 27 bp, synthetic construct
  misc_feature 1..27: note = primer
  source 1..27: mol_type = other DNA
agatgtgaactttgtccccataaggat

SEQ ID NO: 133 - DNA, 29 bp, synthetic construct
  misc_feature 1..29: note = primer
  source 1..29: mol_type = other DNA
acttgctacatttgtgatgaacaaggaag

SEQ ID NO: 134 - DNA, 31 bp, synthetic construct
  misc_feature 1..31: note = primer
  source 1..31: mol_type = other DNA
aaatataaagagaaggacaaacacaaacaga

SEQ ID NO: 135 - DNA, 30 bp, synthetic construct
  misc_feature 1..30: note = primer
  source 1..30: mol_type = other DNA
acttatacaagcactagcaacaactctata

SEQ ID NO: 136 - DNA, 27 bp, synthetic construct
  misc_feature 1..27: note = primer
  source 1..27: mol_type = other DNA
gcaaatactctatctggatcttctctc

SEQ ID NO: 137 - DNA, 26 bp, synthetic construct
  misc_feature 1..26: note = primer
  source 1..26: mol_type = other DNA
gatttggagttccatggagtgatgag

SEQ ID NO: 138 - DNA, 27 bp, synthetic construct
  misc_feature 1..27: note = primer
  source 1..27: mol_type = other DNA
gattctgtttcactgaggccatctatc

SEQ ID NO: 139 - DNA, 23 bp, synthetic construct
  misc_feature 1..23: note = primer
  source 1..23: mol_type = other DNA
cacatctccatcccccagcctga

SEQ ID NO: 140 - DNA, 24 bp, synthetic construct
  misc_feature 1..24: note = primer
  source 1..24: mol_type = other DNA
gtggccaacatgagtgctaaggac

SEQ ID NO: 141 - DNA, 31 bp, synthetic construct
  misc_feature 1..31: note = primer
  source 1..31: mol_type = other DNA
ttatcaagtgggaatcctgtatatgaaaaat

SEQ ID NO: 142 - DNA, 27 bp, synthetic construct
  misc_feature 1..27: note = primer
  source 1..27: mol_type = other DNA
catgataccagtagtcctttgctaatc

SEQ ID NO: 143 - DNA, 27 bp, synthetic construct
  misc_feature 1..27: note = primer
  source 1..27: mol_type = other DNA
agtcgaaaggacaaagaacgccttaag

SEQ ID NO: 144 - DNA, 23 bp, synthetic construct
  misc_feature 1..23: note = primer
  source 1..23: mol_type = other DNA
gtctcctcctcggcttcctcttc

SEQ ID NO: 145 - DNA, 24 bp, synthetic construct
  misc_feature 1..24: note = primer
  source 1..24: mol_type = other DNA
gtgttttctctggctggctctacc

SEQ ID NO: 146 - DNA, 23 bp, synthetic construct
  misc_feature 1..23: note = primer
  source 1..23: mol_type = other DNA
ggtgaaggttgccgaactgtccc

SEQ ID NO: 147 - DNA, 23 bp, synthetic construct
  misc_feature 1..23: note = primer
  source 1..23: mol_type = other DNA
gaagctatgagggaccctgtgag

SEQ ID NO: 148 - DNA, 24 bp, synthetic construct
  misc_feature 1..24: note = primer
  source 1..24: mol_type = other DNA
gcacggacacttgctagtatgttg

SEQ ID NO: 149 - DNA, 24 bp, synthetic construct
  misc_feature 1..24: note = primer
  source 1..24: mol_type = other DNA
gcatcagcttctggtgatgtgagc

SEQ ID NO: 150 - DNA, 23 bp, synthetic construct
  misc_feature 1..23: note = primer
  source 1..23: mol_type = other DNA
gagatgacgcattcatggcctcc

SEQ ID NO: 151 - DNA, 24 bp, synthetic construct
  misc_feature 1..24: note = primer
  source 1..24: mol_type = other DNA
gagtctcagcagtccaattttggc

SEQ ID NO: 152 - DNA, 26 bp, synthetic construct
  misc_feature 1..26: note = primer
  source 1..26: mol_type = other DNA
atatttaacaccgtgcccgatatgcc

SEQ ID NO: 153 - DNA, 27 bp, synthetic construct
  misc_feature 1..27: note = primer
  source 1..27: mol_type = other DNA
aagatggcagtgaacgtatactcaacg

SEQ ID NO: 154 - DNA, 30 bp, synthetic construct
  misc_feature 1..30: note = primer
  source 1..30: mol_type = other DNA
ataattcctgtggacaaattagtaaaagga

SEQ ID NO: 155 - DNA, 30 bp, synthetic construct
  misc_feature 1..30: note = primer
  source 1..30: mol_type = other DNA
gtcaacgtattgaaacttactgttgaagac

SEQ ID NO: 156 - DNA, 24 bp, synthetic construct
  misc_feature 1..24: note = primer
  source 1..24: mol_type = other DNA
gacattgacaagcagtacgtgggc

SEQ ID NO: 157 - DNA, 23 bp, synthetic construct
  misc_feature 1..23: note = primer
  source 1..23: mol_type = other DNA
gaggcacgcggacctccagtggc

SEQ ID NO: 158 - DNA, 27 bp, synthetic construct
  misc_feature 1..27: note = primer
  source 1..27: mol_type = other DNA
ccttgaaaagatcttttgaggtcgagg

SEQ ID NO: 159 - DNA, 31 bp, synthetic construct
  misc_feature 1..31: note = primer
  source 1..31: mol_type = other DNA
gaaaaaaaccttgaagataacttacagagtt

SEQ ID NO: 160 - DNA, 26 bp, synthetic construct
  misc_feature 1..26: note = primer
  source 1..26: mol_type = other DNA
gagagtagatctggagaaaccaacag

SEQ ID NO: 161 - DNA, 23 bp, synthetic construct
  misc_feature 1..23: note = primer
  source 1..23: mol_type = other DNA
gagatgacctggcttccaccact

SEQ ID NO: 162 - DNA, 23 bp, synthetic construct
  misc_feature 1..23: note = primer
  source 1..23: mol_type = other DNA
caggcagctcagagaacggctct

SEQ ID NO: 163 - DNA, 31 bp, synthetic construct
  misc_feature 1..31: note = primer
  source 1..31: mol_type = other DNA
atttttgatcaccatactgaagaggatatag

SEQ ID NO: 164 - DNA, 23 bp, synthetic construct
  misc_feature 1..23: note = primer
  source 1..23: mol_type = other DNA
aactccatccggcacaacctgtc

SEQ ID NO: 165 - DNA, 27 bp, synthetic construct
  misc_feature 1..27: note = primer
  source 1..27: mol_type = other DNA
ctccagggttccttgaaaagaaaacag

SEQ ID NO: 166 - DNA, 24 bp, synthetic construct
  misc_feature 1..24: note = primer
  source 1..24: mol_type = other DNA
gatcaacactctgtggtaggccag

SEQ ID NO: 167 - DNA, 28 bp, synthetic construct
  misc_feature 1..28: note = primer
  source 1..28: mol_type = other DNA
caagttggaattgacagaggtgatatac

SEQ ID NO: 168 - DNA, 23 bp, synthetic construct
  misc_feature 1..23: note = primer
  source 1..23: mol_type = other DNA
gcccctagcagtcttcttgatgc

SEQ ID NO: 169 - DNA, 26 bp, synthetic construct
  misc_feature 1..26: note = primer
  source 1..26: mol_type = other DNA
caacctcataatattctgcagaggcg

SEQ ID NO: 170 - DNA, 23 bp, synthetic construct
  misc_feature 1..23: note = primer
  source 1..23: mol_type = other DNA
gacatgcggaagcacgtggccat

SEQ ID NO: 171 - DNA, 23 bp, synthetic construct
  misc_feature 1..23: note = primer
  source 1..23: mol_type = other DNA
ggctacatgcagccgctgaagca

SEQ ID NO: 172 - DNA, 27 bp, synthetic construct
  misc_feature 1..27: note = primer
  source 1..27: mol_type = other DNA
ttctccaaggatgtcctagtaaacatc

SEQ ID NO: 173 - DNA, 24 bp, synthetic construct
  misc_feature 1..24: note = primer
  source 1..24: mol_type = other DNA
caaagcatgcgtgagaacaaggag

SEQ ID NO: 174 - DNA, 25 bp, synthetic construct
  misc_feature 1..25: note = primer
  source 1..25: mol_type = other DNA
ccagaagtcattggatctgtgtcac

SEQ ID NO: 175 - DNA, 30 bp, synthetic construct
  misc_feature 1..30: note = primer
  source 1..30: mol_type = other DNA
gtaaccatggagcttattacagataacaaa

SEQ ID NO: 176 - DNA, 24 bp, synthetic construct
  misc_feature 1..24: note = primer
  source 1..24: mol_type = other DNA
gtgattcctgtctctctgtcttcc

SEQ ID NO: 177 - DNA, 23 bp, synthetic construct
  misc_feature 1..23: note = primer
  source 1..23: mol_type = other DNA
gcagaggaccgaggaaatggact

SEQ ID NO: 178 - DNA, 24 bp, synthetic construct
  misc_feature 1..24: note = primer
  source 1..24: mol_type = other DNA
gatggagctgtagttacaccctcc

SEQ ID NO: 179 - DNA, 24 bp, synthetic construct
  misc_feature 1..24: note = primer
  source 1..24: mol_type = other DNA
agtcccaagagtggcccaaaagag

SEQ ID NO: 180 - DNA, 25 bp, synthetic construct
  misc_feature 1..25: note = primer
  source 1..25: mol_type = other DNA
gacactcaatcacttgtcggaagtc

SEQ ID NO: 181 - DNA, 23 bp, synthetic construct
  misc_feature 1..23: note = primer
  source 1..23: mol_type = other DNA
gtggaacggccgccttctccatt

SEQ ID NO: 182 - DNA, 23 bp, synthetic construct
  misc_feature 1..23: note = primer
  source 1..23: mol_type = other DNA
gtgaatgaggcctctggggatgg

SEQ ID NO: 183 - DNA, 23 bp, synthetic construct
  misc_feature 1..23: note = primer
  source 1..23: mol_type = other DNA
aagcctggaatggtcccccctcc

SEQ ID NO: 184 - DNA, 23 bp, synthetic construct
  misc_feature 1..23: note = primer
  source 1..23: mol_type = other DNA
aactcgatccgccacaacctgtc

SEQ ID NO: 185 - DNA, 28 bp, synthetic construct
  misc_feature 1..28: note = primer
  source 1..28: mol_type = other DNA
caacagccaactcagtttataaatccag

SEQ ID NO: 186 - DNA, 25 bp, synthetic construct
  misc_feature 1..25: note = primer
  source 1..25: mol_type = other DNA
ctgcagaagaaagatcagcaactgg

SEQ ID NO: 187 - DNA, 26 bp, synthetic construct
  misc_feature 1..26: note = primer
  source 1..26: mol_type = other DNA
atgggaataactgggaacacaagtcc

SEQ ID NO: 188 - DNA, 27 bp, synthetic construct
  misc_feature 1..27: note = primer
  source 1..27: mol_type = other DNA
tctcagatgcaaacatcagtgggaatt

SEQ ID NO: 189 - DNA, 25 bp, synthetic construct
  misc_feature 1..25: note = primer
  source 1..25: mol_type = other DNA
atgatggaggaggatttgcaaggag

SEQ ID NO: 190 - DNA, 25 bp, synthetic construct
  misc_feature 1..25: note = primer
  source 1..25: mol_type = other DNA
gtgagtacccagaacatgaagatgg

SEQ ID NO: 191 - DNA, 23 bp, synthetic construct
  misc_feature 1..23: note = primer
  source 1..23: mol_type = other DNA
ggcctggagcaggatgtcctcca

SEQ ID NO: 192 - DNA, 27 bp, synthetic construct
  misc_feature 1..27: note = primer
  source 1..27: mol_type = other DNA
aatcagatgggtgactcaaatatctcc

SEQ ID NO: 193 - DNA, 24 bp, synthetic construct
  misc_feature 1..24: note = primer
  source 1..24: mol_type = other DNA
tctgctccatctggacacaagcat

SEQ ID NO: 194 - DNA, 23 bp, synthetic construct
  misc_feature 1..23: note = primer
  source 1..23: mol_type = other DNA
ctggagaatgctggaggagacct

SEQ ID NO: 195 - DNA, 26 bp, synthetic construct
  misc_feature 1..26: note = primer
  source 1..26: mol_type = other DNA
aatactctggagcagtgcaatgtgtg

SEQ ID NO: 196 - DNA, 26 bp, synthetic construct
  misc_feature 1..26: note = primer
  source 1..26: mol_type = other DNA
aatactctggagcagtgcaatgtgtg

SEQ ID NO: 197 - DNA, 27 bp, synthetic construct
  misc_feature 1..27: note = primer
  source 1..27: mol_type = other DNA
agagaaatagcccggaaacttgcaaat

SEQ ID NO: 198 - DNA, 29 bp, synthetic construct
  misc_feature 1..29: note = primer
  source 1..29: mol_type = other DNA
aatgaagagcttcgaaacttgtctttgtc

SEQ ID NO: 199 - DNA, 24 bp, synthetic construct
  misc_feature 1..24: note = primer
  source 1..24: mol_type = other DNA
ccacagcgtcctgtgtttactcat

SEQ ID NO: 200 - DNA, 30 bp, synthetic construct
  misc_feature 1..30: note = primer
  source 1..30: mol_type = other DNA
ttccaaaagctgagacaagatcttgaaatg

SEQ ID NO: 201 - DNA, 24 bp, synthetic construct
  misc_feature 1..24: note = primer
  source 1..24: mol_type = other DNA
gtgacgacgtcatcaggaagcaag

SEQ ID NO: 202 - DNA, 23 bp, synthetic construct
  misc_feature 1..23: note = primer
  source 1..23: mol_type = other DNA
gacaacagccggcgtgtggagca

SEQ ID NO: 203 - DNA, 24 bp, synthetic construct
  misc_feature 1..24: note = primer
  source 1..24: mol_type = other DNA
gacttcctgacagacctgatgatg

SEQ ID NO: 204 - DNA, 30 bp, synthetic construct
  misc_feature 1..30: note = primer
  source 1..30: mol_type = other DNA
gatcagtttgacaacttagaaaaacacaca

SEQ ID NO: 205 - DNA, 27 bp, synthetic construct
  misc_feature 1..27: note = primer
  source 1..27: mol_type = other DNA
gctacagacaagagaaaagctttagag

SEQ ID NO: 206 - DNA, 28 bp, synthetic construct
  misc_feature 1..28: note = primer
  source 1..28: mol_type = other DNA
actgtggatattcataaggagaaagtgg

SEQ ID NO: 207 - DNA, 30 bp, synthetic construct
  misc_feature 1..30: note = primer
  source 1..30: mol_type = other DNA
gtcaagtacaaaagagattttgaagaaagc

SEQ ID NO: 208 - DNA, 31 bp, synthetic construct
  misc_feature 1..31: note = primer
  source 1..31: mol_type = other DNA
gtaaaataccatgaagattttgaaaaaacaa

SEQ ID NO: 209 - DNA, 23 bp, synthetic construct
  misc_feature 1..23: note = primer
  source 1..23: mol_type = other DNA
aagcggtaccgcgcggtgtatga

SEQ ID NO: 210 - DNA, 23 bp, synthetic construct
  misc_feature 1..23: note = primer
  source 1..23: mol_type = other DNA
ctggtcagtgagaaggtcggagg

SEQ ID NO: 211 - DNA, 27 bp, synthetic construct
  misc_feature 1..27: note = primer
  source 1..27: mol_type = other DNA
ttctccaatatccccttcttcatcttc

SEQ ID NO: 212 - DNA, 23 bp, synthetic construct
  misc_feature 1..23: note = primer
  source 1..23: mol_type = other DNA
ggccctcctcaggacctgtctgt

SEQ ID NO: 213 - DNA, 23 bp, synthetic construct
  misc_feature 1..23: note = primer
  source 1..23: mol_type = other DNA
ggcagcaaggagcgcttccactg

SEQ ID NO: 214 - DNA, 24 bp, synthetic construct
  misc_feature 1..24: note = primer
  source 1..24: mol_type = other DNA
acctacatcggctctgtgctcatc

SEQ ID NO: 215 - DNA, 23 bp, synthetic construct
  misc_feature 1..23: note = primer
  source 1..23: mol_type = other DNA
accttcacggcatggtgcaactc

SEQ ID NO: 216 - DNA, 23 bp, synthetic construct
  misc_feature 1..23: note = primer
  source 1..23: mol_type = other DNA
cctatgggctatgggcctcgtat

SEQ ID NO: 217 - DNA, 25 bp, synthetic construct
  misc_feature 1..25: note = primer
  source 1..25: mol_type = other DNA
gcacaaatgtctagttcttcctgcc

SEQ ID NO: 218 - DNA, 24 bp, synthetic construct
  misc_feature 1..24: note = primer
  source 1..24: mol_type = other DNA
ctttcccagccagctgtaagcatt

SEQ ID NO: 219 - DNA, 28 bp, synthetic construct
  misc_feature 1..28: note = primer
  source 1..28: mol_type = other DNA
gaaacaatgaccgataaaacagagaagg

SEQ ID NO: 220 - DNA, 46 bp, synthetic construct
  misc_feature 1..46: note = primer
  source 1..46: mol_type = other DNA
gtgccagcaagatccaatctagagcagatggccagtcaggcaccag

SEQ ID NO: 221 - DNA, 52 bp, synthetic construct
  misc_feature 1..52: note = primer
  source 1..52: mol_type = other DNA
gtgccagcaagatccaatctagatcagttatcatctggtgacaaagcttcag

SEQ ID NO: 222 - DNA, 50 bp, synthetic construct
  misc_feature 1..50: note = primer
  source 1..50: mol_type = other DNA
gtgccagcaagatccaatctagacgtcttctaatttcactgctgcacaag

SEQ ID NO: 223 - DNA, 47 bp, synthetic construct
  misc_feature 1..47: note = primer
  source 1..47: mol_type = other DNA
gtgccagcaagatccaatctagaggttcagcttttgccaagcttcag

SEQ ID NO: 224 - DNA, 48 bp, synthetic construct
  misc_feature 1..48: note = primer
  source 1..48: mol_type = other DNA
gtgccagcaagatccaatctagaggctttggatccacagctacctcaa

SEQ ID NO: 225 - DNA, 49 bp, synthetic construct
  misc_feature 1..49: note = primer
  source 1..49: mol_type = other DNA
gtgccagcaagatccaatctagatgggttttcctctccaaacaaaacag

SEQ ID NO: 226 - DNA, 46 bp, synthetic construct
  misc_feature 1..46: note = primer
  source 1..46: mol_type = other DNA
gtgccagcaagatccaatctagatggttttggatcaggcacaggag

SEQ ID NO: 227 - DNA, 49 bp, synthetic construct
  misc_feature 1..49: note = primer
  source 1..49: mol_type = other DNA
attatgaactattaacagaaaatgacatgttccaacccttagggaaccc

SEQ ID NO: 228 - DNA, 47 bp, synthetic construct
  misc_feature 1..47: note = primer
  source 1..47: mol_type = other DNA
ttcttcaggagagaataccatgggtacctccaacccttagggaaccc

SEQ ID NO: 229 - DNA, 49 bp, synthetic construct
  misc_feature 1..49: note = primer
  source 1..49: mol_type = other DNA
gaaattgaacttagctcattaagggaagcttccaacccttagggaaccc

SEQ ID NO: 230 - DNA, 49 bp, synthetic construct
  misc_feature 1..49: note = primer
  source 1..49: mol_type = other DNA
cgagaaaatgtcattgaatataaacactgttccaacccttagggaaccc

SEQ ID NO: 231 - DNA, 53 bp, synthetic construct
  misc_feature 1..53: note = primer
  source 1..53: mol_type = other DNA
gtgccagcaagatccaatctagacctagtgagagccttgctactactgatgat

SEQ ID NO: 232 - DNA, 53 bp, synthetic construct
  misc_feature 1..53: note = primer
  source 1..53: mol_type = other DNA
gtgccagcaagatccaatctagaactgaatctccagtgttagtgaatgactat

SEQ ID NO: 233 - DNA, 53 bp, synthetic construct
  misc_feature 1..53: note = primer
  source 1..53: mol_type = other DNA
gtgccagcaagatccaatctagacagtgcatattagtggacagcacttagtag

SEQ ID NO: 234 - DNA, 51 bp, synthetic construct
  misc_feature 1..51: note = primer
  source 1..51: mol_type = other DNA
gtgccagcaagatccaatctagactgagaatgcacttactggctcattcag

SEQ ID NO: 235 - DNA, 49 bp, synthetic construct
  misc_feature 1..49: note = primer
  source 1..49: mol_type = other DNA
gtgccagcaagatccaatctagaggagacacacaggcagacccatactg

SEQ ID NO: 236 - DNA, 51 bp, synthetic construct
  misc_feature 1..51: note = primer
  source 1..51: mol_type = other DNA
gtgccagcaagatccaatctagaatcaccattgcttggaagtttgattctc

SEQ ID NO: 237 - DNA, 49 bp, synthetic construct
  misc_feature 1..49: note = primer
  source 1..49: mol_type = other DNA
gtgccagcaagatccaatctagaaccagttccctgcgagtctgctactg

SEQ ID NO: 238 - DNA, 50 bp, synthetic construct
  misc_feature 1..50: note = primer
  source 1..50: mol_type = other DNA
gtgccagcaagatccaatctagaattacctggtcatgatcattgtccgtg

SEQ ID NO: 239 - DNA, 45 bp, synthetic construct
  misc_feature 1..45: note = primer
  source 1..45: mol_type = other DNA
tgctacagttgaaactccagcagcgctccaacccttagggaaccc

SEQ ID NO: 240 - DNA, 45 bp, synthetic construct
  misc_feature 1..45: note = primer
  source 1..45: mol_type = other DNA
ctttgaaaagtccagccgcatttcattccaacccttagggaaccc

SEQ ID NO: 241 - DNA, 53 bp, synthetic construct
  misc_feature 1..53: note = primer
  source 1..53: mol_type = other DNA
gtgccagcaagatccaatctagagaagtcacaatgaaacagatttgcaaaaag

SEQ ID NO: 242 - DNA, 44 bp, synthetic construct
  misc_feature 1..44: note = primer
  source 1..44: mol_type = other DNA
gaaattcggcgccttcatcagtatgtccaacccttagggaaccc

SEQ ID NO: 243 - DNA, 53 bp, synthetic construct
  misc_feature 1..53: note = primer
  source 1..53: mol_type = other DNA
gtgccagcaagatccaatctagaaagaagatgaagagtcagatgatgctgatg

SEQ ID NO: 244 - DNA, 49 bp, synthetic construct
  misc_feature 1..49: note = primer
  source 1..49: mol_type = other DNA
attttggatcattgtttgacttggaaaatgtccaacccttagggaaccc

SEQ ID NO: 245 - DNA, 45 bp, synthetic construct
  misc_feature 1..45: note = primer
  source 1..45: mol_type = other DNA
atgggaataactgggaacacaagtcctccaacccttagggaaccc

SEQ ID NO: 246 - DNA, 46 bp, synthetic construct
  misc_feature 1..46: note = primer
  source 1..46: mol_type = other DNA
gtgccagcaagatccaatctagagcttggtgcaggatttggaacag

SEQ ID NO: 247 - DNA, 45 bp, synthetic construct
  misc_feature 1..45: note = primer
  source 1..45: mol_type = other DNA
gtgccagcaagatccaatctagaggagccccccaggccccagtag

SEQ ID NO: 248 - DNA, 51 bp, synthetic construct
  misc_feature 1..51: note = primer
  source 1..51: mol_type = other DNA
gtgccagcaagatccaatctagagatgtcagaccctaagaagaaggaagag

SEQ ID NO: 249 - DNA, 47 bp, synthetic construct
  misc_feature 1..47: note = primer
  source 1..47: mol_type = other DNA
gtgccagcaagatccaatctagaagccaatggagcattcatgcccaa

SEQ ID NO: 250 - DNA, 42 bp, synthetic construct
  misc_feature 1..42: note = primer
  source 1..42: mol_type = other DNA
ctgtgcggtcagagaagaaacgctccaacccttagggaaccc

SEQ ID NO: 251 - DNA, 46 bp, synthetic construct
  misc_feature 1..46: note = primer
  source 1..46: mol_type = other DNA
ttgatagagaaaaacaacccagcgaagtccaacccttagggaaccc

SEQ ID NO: 252 - DNA, 43 bp, synthetic construct
  misc_feature 1..43: note = primer
  source 1..43: mol_type = other DNA
ataacccagcagccaactggcttctccaacccttagggaaccc

SEQ ID NO: 253 - DNA, 46 bp, synthetic construct
  misc_feature 1..46: note = primer
  source 1..46: mol_type = other DNA
gatggtaaattgaaaaaacccaagaattccaacccttagggaaccc

SEQ ID NO: 254 - DNA, 46 bp, synthetic construct
  misc_feature 1..46: note = primer
  source 1..46: mol_type = other DNA
ttcttgatgaagcagatagaatcttggtccaacccttagggaaccc

SEQ ID NO: 255 - DNA, 42 bp, synthetic construct
  misc_feature 1..42: note = primer
  source 1..42: mol_type = other DNA
cacccctgccactttggaacagatccaacccttagggaaccc

SEQ ID NO: 256 - DNA, 48 bp, synthetic construct
  misc_feature 1..48: note = primer
  source 1..48: mol_type = other DNA
ataatctcagtgataccttgaagaagctgtccaacccttagggaaccc

SEQ ID NO: 257 - DNA, 47 bp, synthetic construct
  misc_feature 1..47: note = primer
  source 1..47: mol_type = other DNA
atacggaaacaagtgaaaaaatccaagctccaacccttagggaaccc

SEQ ID NO: 258 - DNA, 52 bp, synthetic construct
  misc_feature 1..52: note = primer
  source 1..52: mol_type = other DNA
gtgccagcaagatccaatctagaaccaagaggctattcaagatctctgtctg

SEQ ID NO: 259 - DNA, 52 bp, synthetic construct
  misc_feature 1..52: note = primer
  source 1..52: mol_type = other DNA
gtgccagcaagatccaatctagaaccaagaggctattcaagatctctgcatg

SEQ ID NO: 260 - DNA, 49 bp, synthetic construct
  misc_feature 1..49: note = primer
  source 1..49: mol_type = other DNA
gcagtggaggaagtctctttaagaaaatagtccaacccttagggaaccc

SEQ ID NO: 261 - DNA, 46 bp, synthetic construct
  misc_feature 1..46: note = primer
  source 1..46: mol_type = other DNA
gtgccagcaagatccaatctagacttctgccgctgcttctgcacag

SEQ ID NO: 262 - DNA, 42 bp, synthetic construct
  misc_feature 1..42: note = primer
  source 1..42: mol_type = other DNA
gcatggggcggctggttctgctgtccaacccttagggaaccc

SEQ ID NO: 263 - DNA, 47 bp, synthetic construct
  misc_feature 1..47: note = primer
  source 1..47: mol_type = other DNA
gtgccagcaagatccaatctagactgaggacatctggaggaaggctg

SEQ ID NO: 264 - DNA, 44 bp, synthetic construct
  misc_feature 1..44: note = primer
  source 1..44: mol_type = other DNA
gtgccagcaagatccaatctagacagcaggaggactccagcgag

SEQ ID NO: 265 - DNA, 43 bp, synthetic construct
  misc_feature 1..43: note = primer
  source 1..43: mol_type = other DNA
gcttcctgctgaactccaagttcctccaacccttagggaaccc

SEQ ID NO: 266 - DNA, 42 bp, synthetic construct
  misc_feature 1..42: note = primer
  source 1..42: mol_type = other DNA
gacttccagccactgcgctattttccaacccttagggaaccc

SEQ ID NO: 267 - DNA, 53 bp, synthetic construct
  misc_feature 1..53: note = primer
  source 1..53: mol_type = other DNA
gtgccagcaagatccaatctagaatccctgtaaaacaaaaaccaaaagaaaag

SEQ ID NO: 268 - DNA, 51 bp, synthetic construct
  misc_feature 1..51: note = primer
  source 1..51: mol_type = other DNA
gtgccagcaagatccaatctagaagtccacaggatcagagtggactttaag

SEQ ID NO: 269 - DNA, 50 bp, synthetic construct
  misc_feature 1..50: note = primer
  source 1..50: mol_type = other DNA
gtgccagcaagatccaatctagactctgtgccagtagtgggcatgtagag

SEQ ID NO: 270 - DNA, 49 bp, synthetic construct
  misc_feature 1..49: note = primer
  source 1..49: mol_type = other DNA
gtgccagcaagatccaatctagagtggaaggcaacatcaggctacaaag

SEQ ID NO: 271 - DNA, 45 bp, synthetic construct
  misc_feature 1..45: note = primer
  source 1..45: mol_type = other DNA
cagacctactccaatgaagtccattgtccaacccttagggaaccc

SEQ ID NO: 272 - DNA, 42 bp, synthetic construct
  misc_feature 1..42: note = primer
  source 1..42: mol_type = other DNA
gaaatgacccattcatggccgcctccaacccttagggaaccc

SEQ ID NO: 273 - DNA, 45 bp, synthetic construct
  misc_feature 1..45: note = primer
  source 1..45: mol_type = other DNA
gactctcagcatgtcagttctgtaactccaacccttagggaaccc

SEQ ID NO: 274 - DNA, 45 bp, synthetic construct
  misc_feature 1..45: note = primer
  source 1..45: mol_type = other DNA
cctgagcctccaacaacaaacaaatgtccaacccttagggaaccc

SEQ ID NO: 275 - DNA, 46 bp, synthetic construct
  misc_feature 1..46: note = primer
  source 1..46: mol_type = other DNA
gatgagcaattcttaggttttggctcatccaacccttagggaaccc

SEQ ID NO: 276 - DNA, 44 bp, synthetic construct
  misc_feature 1..44: note = primer
  source 1..44: mol_type = other DNA
gaagaacctaggaaagtccgctttgtccaacccttagggaaccc

SEQ ID NO: 277 - DNA, 46 bp, synthetic construct
  misc_feature 1..46: note = primer
  source 1..46: mol_type = other DNA
gaccctaataggagtattcataccagctccaacccttagggaaccc

SEQ ID NO: 278 - DNA, 42 bp, synthetic construct
  misc_feature 1..42: note = primer
  source 1..42: mol_type = other DNA
tctgaacaacccagtcctgccagtccaacccttagggaaccc

SEQ ID NO: 279 - DNA, 50 bp, synthetic construct
  misc_feature 1..50: note = primer
  source 1..50: mol_type = other DNA
attcttgaagtgaaaagtccaataaagcaaatccaacccttagggaaccc

SEQ ID NO: 280 - DNA, 44 bp, synthetic construct
  misc_feature 1..44: note = primer
  source 1..44: mol_type = other DNA
gcatacctagatgaactggtagagctccaacccttagggaaccc

SEQ ID NO: 281 - DNA, 42 bp, synthetic construct
  misc_feature 1..42: note = primer
  source 1..42: mol_type = other DNA
tgcaccgtccaggtgaggttagatccaacccttagggaaccc

SEQ ID NO: 282 - DNA, 42 bp, synthetic construct
  misc_feature 1..42: note = primer
  source 1..42: mol_type = other DNA
gaggagccgaggaaggtctgctttccaacccttagggaaccc

SEQ ID NO: 283 - DNA, 42 bp, synthetic construct
  misc_feature 1..42: note = primer
  source 1..42: mol_type = other DNA
gtgatggtaatgcccgaaggagctccaacccttagggaaccc

SEQ ID NO: 284 - DNA, 42 bp, synthetic construct
  misc_feature 1..42: note = primer
  source 1..42: mol_type = other DNA
gacgccaacaaggagagcagcaatccaacccttagggaaccc

SEQ ID NO: 285 - DNA, 42 bp, synthetic construct
  misc_feature 1..42: note = primer
  source 1..42: mol_type = other DNA
tctgcccagtcaagcccgtccaatccaacccttagggaaccc

SEQ ID NO: 286 - DNA, 46 bp, synthetic construct
  misc_feature 1..46: note = primer
  source 1..46: mol_type = other DNA
agatgtgaactttgtccccataaggattccaacccttagggaaccc

SEQ ID NO: 287 - DNA, 48 bp, synthetic construct
  misc_feature 1..48: note = primer
  source 1..48: mol_type = other DNA
acttgctacatttgtgatgaacaaggaagtccaacccttagggaaccc

SEQ ID NO: 288 - DNA, 50 bp, synthetic construct
  misc_feature 1..50: note = primer
  source 1..50: mol_type = other DNA
aaatataaagagaaggacaaacacaaacagatccaacccttagggaaccc

SEQ ID NO: 289 - DNA, 49 bp, synthetic construct
  misc_feature 1..49: note = primer
  source 1..49: mol_type = other DNA
acttatacaagcactagcaacaactctatatccaacccttagggaaccc

SEQ ID NO: 290 - DNA, 46 bp, synthetic construct
  misc_feature 1..46: note = primer
  source 1..46: mol_type = other DNA
gcaaatactctatctggatcttctctctccaacccttagggaaccc

SEQ ID NO: 291 - DNA, 45 bp, synthetic construct
  misc_feature 1..45: note = primer
  source 1..45: mol_type = other DNA
gatttggagttccatggagtgatgagtccaacccttagggaaccc

SEQ ID NO: 292 - DNA, 46 bp, synthetic construct
  misc_feature 1..46: note = primer
  source 1..46: mol_type = other DNA
gattctgtttcactgaggccatctatctccaacccttagggaaccc

SEQ ID NO: 293 - DNA, 42 bp, synthetic construct
  misc_feature 1..42: note = primer
  source 1..42: mol_type = other DNA
cacatctccatcccccagcctgatccaacccttagggaaccc

SEQ ID NO: 294 - DNA, 43 bp, synthetic construct
  misc_feature 1..43: note = primer
  source 1..43: mol_type = other DNA
gtggccaacatgagtgctaaggactccaacccttagggaaccc

SEQ ID NO: 295 - DNA, 50 bp, synthetic construct
  misc_feature 1..50: note = primer
  source 1..50: mol_type = other DNA
ttatcaagtgggaatcctgtatatgaaaaattccaacccttagggaaccc

SEQ ID NO: 296 - DNA, 46 bp, synthetic construct
  misc_feature 1..46: note = primer
  source 1..46: mol_type = other DNA
catgataccagtagtcctttgctaatctccaacccttagggaaccc

SEQ ID NO: 297 - DNA, 46 bp, synthetic construct
  misc_feature 1..46: note = primer
  source 1..46: mol_type = other DNA
agtcgaaaggacaaagaacgccttaagtccaacccttagggaaccc

SEQ ID NO: 298 - DNA, 42 bp, synthetic construct
  misc_feature 1..42: note = primer
  source 1..42: mol_type = other DNA
gtctcctcctcggcttcctcttctccaacccttagggaaccc

SEQ ID NO: 299 - DNA, 43 bp, synthetic construct
  misc_feature 1..43: note = primer
  source 1..43: mol_type = other DNA
gtgttttctctggctggctctacctccaacccttagggaaccc

SEQ ID NO: 300 - DNA, 42 bp, synthetic construct
  misc_feature 1..42: note = primer
  source 1..42: mol_type = other DNA
ggtgaaggttgccgaactgtccctccaacccttagggaaccc

SEQ ID NO: 301 - DNA, 42 bp, synthetic construct
  misc_feature 1..42: note = primer
  source 1..42: mol_type = other DNA
gaagctatgagggaccctgtgagtccaacccttagggaaccc

SEQ ID NO: 302 - DNA, 43 bp, synthetic construct
  misc_feature 1..43: note = primer
  source 1..43: mol_type = other DNA
gcacggacacttgctagtatgttgtccaacccttagggaaccc

SEQ ID NO: 303 - DNA, 43 bp, synthetic construct
  misc_feature 1..43: note = primer
  source 1..43: mol_type = other DNA
gcatcagcttctggtgatgtgagctccaacccttagggaaccc

SEQ ID NO: 304 - DNA, 42 bp, synthetic construct
  misc_feature 1..42: note = primer
  source 1..42: mol_type = other DNA
gagatgacgcattcatggcctcctccaacccttagggaaccc

SEQ ID NO: 305 - DNA, 43 bp, synthetic construct
  misc_feature 1..43: note = primer
  source 1..43: mol_type = other DNA
gagtctcagcagtccaattttggctccaacccttagggaaccc

SEQ ID NO: 306 - DNA, 45 bp, synthetic construct
  misc_feature 1..45: note = primer
  source 1..45: mol_type = other DNA
atatttaacaccgtgcccgatatgcctccaacccttagggaaccc

SEQ ID NO: 307 - DNA, 46 bp, synthetic construct
  misc_feature 1..46: note = primer
  source 1..46: mol_type = other DNA
aagatggcagtgaacgtatactcaacgtccaacccttagggaaccc

SEQ ID NO: 308 - DNA, 49 bp, synthetic construct
  misc_feature 1..49: note = primer
  source 1..49: mol_type = other DNA
ataattcctgtggacaaattagtaaaaggatccaacccttagggaaccc

SEQ ID NO: 309 - DNA, 49 bp, synthetic construct
  misc_feature 1..49: note = primer
  source 1..49: mol_type = other DNA
gtcaacgtattgaaacttactgttgaagactccaacccttagggaaccc

SEQ ID NO: 310 - DNA, 43 bp, synthetic construct
  misc_feature 1..43: note = primer
  source 1..43: mol_type = other DNA
gacattgacaagcagtacgtgggctccaacccttagggaaccc

SEQ ID NO: 311 - DNA, 42 bp, synthetic construct
  misc_feature 1..42: note = primer
  source 1..42: mol_type = other DNA
gaggcacgcggacctccagtggctccaacccttagggaaccc

SEQ ID NO: 312 - DNA, 46 bp, synthetic construct
  misc_feature 1..46: note = primer
  source 1..46: mol_type = other DNA
ccttgaaaagatcttttgaggtcgaggtccaacccttagggaaccc

SEQ ID NO: 313 - DNA, 50 bp, synthetic construct
  misc_feature 1..50: note = primer
  source 1..50: mol_type = other DNA
gaaaaaaaccttgaagataacttacagagtttccaacccttagggaaccc

SEQ ID NO: 314 - DNA, 45 bp, synthetic construct
  misc_feature 1..45: note = primer
  source 1..45: mol_type = other DNA
gagagtagatctggagaaaccaacagtccaacccttagggaaccc

SEQ ID NO: 315 - DNA, 42 bp, synthetic construct
  misc_feature 1..42: note = primer
  source 1..42: mol_type = other DNA
gagatgacctggcttccaccacttccaacccttagggaaccc

SEQ ID NO: 316 - DNA, 42 bp, synthetic construct
  misc_feature 1..42: note = primer
  source 1..42: mol_type = other DNA
caggcagctcagagaacggctcttccaacccttagggaaccc

SEQ ID NO: 317 - DNA, 50 bp, synthetic construct
  misc_feature 1..50: note = primer
  source 1..50: mol_type = other DNA
atttttgatcaccatactgaagaggatatagtccaacccttagggaaccc

SEQ ID NO: 318 - DNA, 42 bp, synthetic construct
  misc_feature 1..42: note = primer
  source 1..42: mol_type = other DNA
aactccatccggcacaacctgtctccaacccttagggaaccc

SEQ ID NO: 319 - DNA, 46 bp, synthetic construct
  misc_feature 1..46: note = primer
  source 1..46: mol_type = other DNA
ctccagggttccttgaaaagaaaacagtccaacccttagggaaccc

SEQ ID NO: 320 - DNA, 43 bp, synthetic construct
  misc_feature 1..43: note = primer
  source 1..43: mol_type = other DNA
gatcaacactctgtggtaggccagtccaacccttagggaaccc

SEQ ID NO: 321 - DNA, 47 bp, synthetic construct
  misc_feature 1..47: note = primer
  source 1..47: mol_type = other DNA
caagttggaattgacagaggtgatatactccaacccttagggaaccc

SEQ ID NO: 322 - DNA, 42 bp, synthetic construct
  misc_feature 1..42: note = primer
  source 1..42: mol_type = other DNA
gcccctagcagtcttcttgatgctccaacccttagggaaccc

SEQ ID NO: 323 - DNA, 45 bp, synthetic construct
  misc_feature 1..45: note = primer
  source 1..45: mol_type = other DNA
caacctcataatattctgcagaggcgtccaacccttagggaaccc

SEQ ID NO: 324 - DNA, 42 bp, synthetic construct
  misc_feature 1..42: note = primer
  source 1..42: mol_type = other DNA
gacatgcggaagcacgtggccattccaacccttagggaaccc

SEQ ID NO: 325 - DNA, 42 bp, synthetic construct
  misc_feature 1..42: note = primer
  source 1..42: mol_type = other DNA
ggctacatgcagccgctgaagcatccaacccttagggaaccc

SEQ ID NO: 326 - DNA, 46 bp, synthetic construct
  misc_feature 1..46: note = primer
  source 1..46: mol_type = other DNA
ttctccaaggatgtcctagtaaacatctccaacccttagggaaccc

SEQ ID NO: 327 - DNA, 43 bp, synthetic construct
  misc_feature 1..43: note = primer
  source 1..43: mol_type = other DNA
caaagcatgcgtgagaacaaggagtccaacccttagggaaccc

SEQ ID NO: 328 - DNA, 44 bp, synthetic construct
  misc_feature 1..44: note = primer
  source 1..44: mol_type = other DNA
ccagaagtcattggatctgtgtcactccaacccttagggaaccc

SEQ ID NO: 329 - DNA, 49 bp, synthetic construct
  misc_feature 1..49: note = primer
  source 1..49: mol_type = other DNA
gtaaccatggagcttattacagataacaaatccaacccttagggaaccc

SEQ ID NO: 330 - DNA, 43 bp, synthetic construct
  misc_feature 1..43: note = primer
  source 1..43: mol_type = other DNA
gtgattcctgtctctctgtcttcctccaacccttagggaaccc

SEQ ID NO: 331 - DNA, 42 bp, synthetic construct
  misc_feature 1..42: note = primer
  source 1..42: mol_type = other DNA
gcagaggaccgaggaaatggacttccaacccttagggaaccc

SEQ ID NO: 332 - DNA, 43 bp, synthetic construct
  misc_feature 1..43: note = primer
  source 1..43: mol_type = other DNA
gatggagctgtagttacaccctcctccaacccttagggaaccc

SEQ ID NO: 333 - DNA, 43 bp, synthetic construct
  misc_feature 1..43: note = primer
  source 1..43: mol_type = other DNA
agtcccaagagtggcccaaaagagtccaacccttagggaaccc

SEQ ID NO: 334 - DNA, 44 bp, synthetic construct
  misc_feature 1..44: note = primer
  source 1..44: mol_type = other DNA
gacactcaatcacttgtcggaagtctccaacccttagggaaccc

SEQ ID NO: 335 - DNA, 42 bp, synthetic construct
  misc_feature 1..42: note = primer
  source 1..42: mol_type = other DNA
gtggaacggccgccttctccatttccaacccttagggaaccc

SEQ ID NO: 336 - DNA, 42 bp, synthetic construct
  misc_feature 1..42: note = primer
  source 1..42: mol_type = other DNA
gtgaatgaggcctctggggatggtccaacccttagggaaccc

SEQ ID NO: 337 - DNA, 42 bp, synthetic construct
  misc_feature 1..42: note = primer
  source 1..42: mol_type = other DNA
aagcctggaatggtcccccctcctccaacccttagggaaccc

SEQ ID NO: 338 - DNA, 42 bp, synthetic construct
  misc_feature 1..42: note = primer
  source 1..42: mol_type = other DNA
aactcgatccgccacaacctgtctccaacccttagggaaccc

SEQ ID NO: 339 - DNA, 47 bp, synthetic construct
  misc_feature 1..47: note = primer
  source 1..47: mol_type = other DNA
caacagccaactcagtttataaatccagtccaacccttagggaaccc

SEQ ID NO: 340 - DNA, 44 bp, synthetic construct
  misc_feature 1..44: note = primer
  source 1..44: mol_type = other DNA
ctgcagaagaaagatcagcaactggtccaacccttagggaaccc

SEQ ID NO: 341 - DNA, 45 bp, synthetic construct; identical to SEQ ID NO: 245
  misc_feature 1..45: note = primer
  source 1..45: mol_type = other DNA

SEQ ID NO: 342 - DNA, 46 bp, synthetic construct
  misc_feature 1..46: note = primer
  source 1..46: mol_type = other DNA
tctcagatgcaaacatcagtgggaatttccaacccttagggaaccc

SEQ ID NO: 343 - DNA, 44 bp, synthetic construct
  misc_feature 1..44: note = primer
  source 1..44: mol_type = other DNA
atgatggaggaggatttgcaaggagtccaacccttagggaaccc

SEQ ID NO: 344 - DNA, 44 bp, synthetic construct
  misc_feature 1..44: note = primer
  source 1..44: mol_type = other DNA
gtgagtacccagaacatgaagatggtccaacccttagggaaccc

SEQ ID NO: 345 - DNA, 42 bp, synthetic construct
  misc_feature 1..42: note = primer
  source 1..42: mol_type = other DNA
ggcctggagcaggatgtcctccatccaacccttagggaaccc

SEQ ID NO: 346 - DNA, 46 bp, synthetic construct
  misc_feature 1..46: note = primer
  source 1..46: mol_type = other DNA
aatcagatgggtgactcaaatatctcctccaacccttagggaaccc

SEQ ID NO: 347 - DNA, 43 bp, synthetic construct
  misc_feature 1..43: note = primer
  source 1..43: mol_type = other DNA
tctgctccatctggacacaagcattccaacccttagggaaccc

SEQ ID NO: 348 - DNA, 42 bp, synthetic construct
  misc_feature 1..42: note = primer
  source 1..42: mol_type = other DNA
ctggagaatgctggaggagaccttccaacccttagggaaccc

SEQ ID NO: 349 - DNA, 45 bp, synthetic construct
  misc_feature 1..45: note = primer
  source 1..45: mol_type = other DNA
aatactctggagcagtgcaatgtgtgtccaacccttagggaaccc

SEQ ID NO: 350 - DNA, 45 bp, synthetic construct; identical to SEQ ID NO: 349
  misc_feature 1..45: note = primer
  source 1..45: mol_type = other DNA

SEQ ID NO: 351 - DNA, 46 bp, synthetic construct
  misc_feature 1..46: note = primer
  source 1..46: mol_type = other DNA
agagaaatagcccggaaacttgcaaattccaacccttagggaaccc

SEQ ID NO: 352 - DNA, 48 bp, synthetic construct
  misc_feature 1..48: note = primer
  source 1..48: mol_type = other DNA
aatgaagagcttcgaaacttgtctttgtctccaacccttagggaaccc

SEQ ID NO: 353 - DNA, 43 bp, synthetic construct
  misc_feature 1..43: note = primer
  source 1..43: mol_type = other DNA
ccacagcgtcctgtgtttactcattccaacccttagggaaccc

SEQ ID NO: 354 - DNA, 49 bp, synthetic construct
  misc_feature 1..49: note = primer
  source 1..49: mol_type = other DNA
ttccaaaagctgagacaagatcttgaaatgtccaacccttagggaaccc

SEQ ID NO: 355 - DNA, 43 bp, synthetic construct
  misc_feature 1..43: note = primer
  source 1..43: mol_type = other DNA
gtgacgacgtcatcaggaagcaagtccaacccttagggaaccc

SEQ ID NO: 356 - DNA, 42 bp, synthetic construct
  misc_feature 1..42: note = primer
  source 1..42: mol_type = other DNA
gacaacagccggcgtgtggagcatccaacccttagggaaccc

SEQ ID NO: 357 - DNA, 43 bp, synthetic construct
  misc_feature 1..43: note = primer
  source 1..43: mol_type = other DNA
gacttcctgacagacctgatgatgtccaacccttagggaaccc

SEQ ID NO: 358 - DNA, 49 bp, synthetic construct
  misc_feature 1..49: note = primer
  source 1..49: mol_type = other DNA
gatcagtttgacaacttagaaaaacacacatccaacccttagggaaccc

SEQ ID NO: 359 - DNA, 46 bp, synthetic construct
  misc_feature 1..46: note = primer
  source 1..46: mol_type = other DNA
gctacagacaagagaaaagctttagagtccaacccttagggaaccc

SEQ ID NO: 360 - DNA, 47 bp, synthetic construct
  misc_feature 1..47: note = primer
  source 1..47: mol_type = other DNA
actgtggatattcataaggagaaagtggtccaacccttagggaaccc

SEQ ID NO: 361 - DNA, 49 bp, synthetic construct
  misc_feature 1..49: note = primer
  source 1..49: mol_type = other DNA
gtcaagtacaaaagagattttgaagaaagctccaacccttagggaaccc

SEQ ID NO: 362 - DNA, 50 bp, synthetic construct
  misc_feature 1..50: note = primer
  source 1..50: mol_type = other DNA
gtaaaataccatgaagattttgaaaaaacaatccaacccttagggaaccc

SEQ ID NO: 363 - DNA, 42 bp, synthetic construct
  misc_feature 1..42: note = primer
  source 1..42: mol_type = other DNA
aagcggtaccgcgcggtgtatgatccaacccttagggaaccc

SEQ ID NO: 364 - DNA, 42 bp, synthetic construct
  misc_feature 1..42: note = primer
  source 1..42: mol_type = other DNA
ctggtcagtgagaaggtcggaggtccaacccttagggaaccc

SEQ ID NO: 365 - DNA, 46 bp, synthetic construct
  misc_feature 1..46: note = primer
  source 1..46: mol_type = other DNA
ttctccaatatccccttcttcatcttctccaacccttagggaaccc

SEQ ID NO: 366 - DNA, 42 bp, synthetic construct
  misc_feature 1..42: note = primer
  source 1..42: mol_type = other DNA
ggccctcctcaggacctgtctgttccaacccttagggaaccc

SEQ ID NO: 367 - DNA, 42 bp, synthetic construct
  misc_feature 1..42: note = primer
  source 1..42: mol_type = other DNA
ggcagcaaggagcgcttccactgtccaacccttagggaaccc

SEQ ID NO: 368 - DNA, 43 bp, synthetic construct
  misc_feature 1..43: note = primer
  source 1..43: mol_type = other DNA
acctacatcggctctgtgctcatctccaacccttagggaaccc

SEQ ID NO: 369 - DNA, 42 bp, synthetic construct
  misc_feature 1..42: note = primer
  source 1..42: mol_type = other DNA
accttcacggcatggtgcaactctccaacccttagggaaccc

SEQ ID NO: 370 - DNA, 42 bp, synthetic construct
  misc_feature 1..42: note = primer
  source 1..42: mol_type = other DNA
cctatgggctatgggcctcgtattccaacccttagggaaccc

SEQ ID NO: 371 - DNA, 44 bp, synthetic construct
  misc_feature 1..44: note = primer
  source 1..44: mol_type = other DNA
gcacaaatgtctagttcttcctgcctccaacccttagggaaccc

SEQ ID NO: 372 - DNA, 43 bp, synthetic construct
  misc_feature 1..43: note = primer
  source 1..43: mol_type = other DNA
ctttcccagccagctgtaagcatttccaacccttagggaaccc

SEQ ID NO: 373 - DNA, 47 bp, synthetic construct
  misc_feature 1..47: note = primer
  source 1..47: mol_type = other DNA
gaaacaatgaccgataaaacagagaaggtccaacccttagggaaccc

SEQ ID NO: 374 - DNA, 22 bp, synthetic construct
  misc_feature 1..22: note = primer
  source 1..22: mol_type = other DNA
agcagcagctacgggcagcaga

SEQ ID NO: 375 - DNA, 22 bp, synthetic construct
  misc_feature 1..22: note = primer
  source 1..22: mol_type = other DNA
gaggaggacgcggtggaatggg

SEQ ID NO: 376 - DNA, 24 bp, synthetic construct
  misc_feature 1..24: note = primer
  source 1..24: mol_type = other DNA
gaggtggcttcaataagcctggtg

SEQ ID NO: 377 - DNA, 27 bp, synthetic construct
  misc_feature 1..27: note = primer
  source 1..27: mol_type = other DNA
tggatgaaggaccagatcttgatctag

SEQ ID NO: 378 - DNA, 28 bp, synthetic construct
  misc_feature 1..28: note = primer
  source 1..28: mol_type = other DNA
gttcactgctggcctataatacaacctc

SEQ ID NO: 379 - DNA, 30 bp, synthetic construct
  misc_feature 1..30: note = primer
  source 1..30: mol_type = other DNA
acccttcttatgactcagtcagaagaggag

SEQ ID NO: 380 - DNA, 23 bp, synthetic construct
  misc_feature 1..23: note = primer
  source 1..23: mol_type = other DNA
gtcctccccttggaggggcacaa

SEQ ID NO: 381 - DNA, 26 bp, synthetic construct
  misc_feature 1..26: note = primer
  source 1..26: mol_type = other DNA
gttattccaggatctttggagacccg

SEQ ID NO: 382 - DNA, 28 bp, synthetic construct
  misc_feature 1..28: note = primer
  source 1..28: mol_type = other DNA
gaagccttatcagttgtgagtgaggacc

SEQ ID NO: 383 - DNA, 24 bp, synthetic construct
  misc_feature 1..24: note = primer
  source 1..24: mol_type = other DNA
atttaccatatgagccccccagga

SEQ ID NO: 384 - DNA, 25 bp, synthetic construct
  misc_feature 1..25: note = primer
  source 1..25: mol_type = other DNA
ctgctcaaccatctccttccacagt

SEQ ID NO: 385 - DNA, 30 bp, synthetic construct
  misc_feature 1..30: note = primer
  source 1..30: mol_type = other DNA
atccttatcagattcttggaccaacaagta

SEQ ID NO: 386 - DNA, 22 bp, synthetic construct
  misc_feature 1..22: note = primer
  source 1..22: mol_type = other DNA
gcagtggccagatccagctttg

SEQ ID NO: 387 - DNA, 23 bp, synthetic construct
  misc_feature 1..23: note = primer
  source 1..23: mol_type = other DNA
atcccgtcggagacggtctcttc

SEQ ID NO: 388 - DNA, 28 bp, synthetic construct
  misc_feature 1..28: note = primer
  source 1..28: mol_type = other DNA
ctcaggtacctgacaatgatgagcagtt

SEQ ID NO: 389 - DNA, 29 bp, synthetic construct
  misc_feature 1..29: note = primer
  source 1..29: mol_type = other DNA
gagacatcaaacaagagccaggaatgtat

SEQ ID NO: 390 - DNA, 22 bp, synthetic construct
  misc_feature 1..22: note = primer
  source 1..22: mol_type = other DNA
atgtcaccgggtgcgcatcaat

SEQ ID NO: 391 - DNA, 24 bp, synthetic construct
  misc_feature 1..24: note = primer
  source 1..24: mol_type = other DNA
attggcaatggcctctcacctcag

SEQ ID NO: 392 - DNA, 30 bp, synthetic construct
  misc_feature 1..30: note = primer
  source 1..30: mol_type = other DNA
aattcaattcgtcataatctgtccctacac

SEQ ID NO: 393 - DNA, 29 bp, synthetic construct
  misc_feature 1..29: note = primer
  source 1..29: mol_type = other DNA
gtgagaaaccataccagtgtgacttcaag

SEQ ID NO: 394 - DNA, 23 bp, synthetic construct
  misc_feature 1..23: note = primer
  source 1..23: mol_type = other DNA
cccaggacagcagcagggctacg

SEQ ID NO: 395 - DNA, 26 bp, synthetic construct
  misc_feature 1..26: note = primer
  source 1..26: mol_type = other DNA
agcagaggccttatggatatgaccag

SEQ ID NO: 396 - DNA, 23 bp, synthetic construct
  misc_feature 1..23: note = primer
  source 1..23: mol_type = other DNA
atcatgcccaagaagccagcaga

SEQ ID NO: 397 - DNA, 25 bp, synthetic construct
  misc_feature 1..25: note = primer
  source 1..25: mol_type = other DNA
gtttcaaagtcaccctcccaccttt

SEQ ID NO: 398 - DNA, 30 bp, synthetic construct
  misc_feature 1..30: note = primer
  source 1..30: mol_type = other DNA
tgttcaagaaggaagtgtatcttcatacat

SEQ ID NO: 399 - DNA, 27 bp, synthetic construct
  misc_feature 1..27: note = primer
  source 1..27: mol_type = other DNA
atgtgcttttccagactgatccaactg

SEQ ID NO: 400 - DNA, 23 bp, synthetic construct
  misc_feature 1..23: note = primer
  source 1..23: mol_type = other DNA
tggaggtggaggtggaggtggag

SEQ ID NO: 401 - DNA, 23 bp, synthetic construct
  misc_feature 1..23: note = primer
  source 1..23: mol_type = other DNA
cgtggaggcagaggtggcatggg

SEQ ID NO: 402 - DNA, 25 bp, synthetic construct
  misc_feature 1..25: note = primer
  source 1..25: mol_type = other DNA
cgtggtggcttcaataaatttggtg

SEQ ID NO: 403 - DNA, 24 bp, synthetic construct
  misc_feature 1..24: note = primer
  source 1..24: mol_type = other DNA
accaaggatcacgtcatgactccg

SEQ ID NO: 404 - DNA, 23 bp, synthetic construct
  misc_feature 1..23: note = primer
  source 1..23: mol_type = other DNA
ggctgccgtggaatggtttgatg

SEQ ID NO: 405 - DNA, 28 bp, synthetic construct
  misc_feature 1..28: note = primer
  source 1..28: mol_type = other DNA
cttctttaagcagtgtggggttgttaag

SEQ ID NO: 406 - DNA, 45 bp, synthetic construct
  misc_feature 1..45: note = primer
  source 1..45: mol_type = other DNA
gtgccagcaagatccaatctagaagcagcagctacgggcagcaga

SEQ ID NO: 407 - DNA, 45 bp, synthetic construct
  misc_feature 1..45: note = primer
  source 1..45: mol_type = other DNA
gtgccagcaagatccaatctagagaggaggacgcggtggaatggg

SEQ ID NO: 408 - DNA, 47 bp, synthetic construct
  misc_feature 1..47: note = primer
  source 1..47: mol_type = other DNA
gtgccagcaagatccaatctagagaggtggcttcaataagcctggtg

SEQ ID NO: 409 - DNA, 50 bp, synthetic construct
  misc_feature 1..50: note = primer
  source 1..50: mol_type = other DNA
gtgccagcaagatccaatctagatggatgaaggaccagatcttgatctag

SEQ ID NO: 410 - DNA, 47 bp, synthetic construct
  misc_feature 1..47: note = primer
  source 1..47: mol_type = other DNA
gttcactgctggcctataatacaacctctccaacccttagggaaccc

SEQ ID NO: 411 - DNA, 49 bp, synthetic construct
  misc_feature 1..49: note = primer
  source 1..49: mol_type = other DNA
acccttcttatgactcagtcagaagaggagtccaacccttagggaaccc

SEQ ID NO: 412 - DNA, 42 bp, synthetic construct
  misc_feature 1..42: note = primer
  source 1..42: mol_type = other DNA
gtcctccccttggaggggcacaatccaacccttagggaaccc

SEQ ID NO: 413 - DNA, 45 bp, synthetic construct
  misc_feature 1..45: note = primer
  source 1..45: mol_type = other DNA
gttattccaggatctttggagacccgtccaacccttagggaaccc

SEQ ID NO: 414 - DNA, 47 bp, synthetic construct
  misc_feature 1..47: note = primer
  source 1..47: mol_type = other DNA
gaagccttatcagttgtgagtgaggacctccaacccttagggaaccc

SEQ ID NO: 415 - DNA, 43 bp, synthetic construct
  misc_feature 1..43: note = primer
  source 1..43: mol_type = other DNA
atttaccatatgagccccccaggatccaacccttagggaaccc

SEQ ID NO: 416 - DNA, 44 bp, synthetic construct
  misc_feature 1..44: note = primer
  source 1..44: mol_type = other DNA
ctgctcaaccatctccttccacagttccaacccttagggaaccc

SEQ ID NO: 417 - DNA, 49 bp, synthetic construct
  misc_feature 1..49: note = primer
  source 1..49: mol_type = other DNA
atccttatcagattcttggaccaacaagtatccaacccttagggaaccc

SEQ ID NO: 418 - DNA, 41 bp, synthetic construct
  misc_feature 1..41: note = primer
  source 1..41: mol_type = other DNA
gcagtggccagatccagctttgtccaacccttagggaaccc

SEQ ID NO: 419 - DNA, 42 bp, synthetic construct
  misc_feature 1..42: note = primer
  source 1..42: mol_type = other DNA
atcccgtcggagacggtctcttctccaacccttagggaaccc

SEQ ID NO: 420 - DNA, 47 bp, synthetic construct
  misc_feature 1..47: note = primer
  source 1..47: mol_type = other DNA
ctcaggtacctgacaatgatgagcagtttccaacccttagggaaccc

SEQ ID NO: 421 - DNA, 48 bp, synthetic construct
  misc_feature 1..48: note = primer
  source 1..48: mol_type = other DNA
gagacatcaaacaagagccaggaatgtattccaacccttagggaaccc

SEQ ID NO: 422 - DNA, 41 bp, synthetic construct
  misc_feature 1..41: note = primer
  source 1..41: mol_type = other DNA
atgtcaccgggtgcgcatcaattccaacccttagggaaccc

SEQ ID NO: 423 - DNA, 47 bp, synthetic construct
  misc_feature 1..47: note = primer
  source 1..47: mol_type = other DNA
gtgccagcaagatccaatctagaattggcaatggcctctcacctcag

SEQ ID NO: 424 - DNA, 49 bp, synthetic construct
  misc_feature 1..49: note = primer
  source 1..49: mol_type = other DNA
aattcaattcgtcataatctgtccctacactccaacccttagggaaccc

SEQ ID NO: 425 - DNA, 48 bp, synthetic construct
  misc_feature 1..48: note = primer
  source 1..48: mol_type = other DNA
gtgagaaaccataccagtgtgacttcaagtccaacccttagggaaccc

SEQ ID NO: 426 - DNA, 46 bp, synthetic construct
  misc_feature 1..46: note = primer
  source 1..46: mol_type = other DNA
gtgccagcaagatccaatctagacccaggacagcagcagggctacg

SEQ ID NO: 427 - DNA, 49 bp, synthetic construct
  misc_feature 1..49: note = primer
  source 1..49: mol_type = other DNA
gtgccagcaagatccaatctagaagcagaggccttatggatatgaccag

SEQ ID NO: 428 - DNA, 42 bp, synthetic construct
  misc_feature 1..42: note = primer
  source 1..42: mol_type = other DNA
atcatgcccaagaagccagcagatccaacccttagggaaccc

SEQ ID NO: 429 - DNA, 44 bp, synthetic construct
  misc_feature 1..44: note = primer
  source 1..44: mol_type = other DNA
gtttcaaagtcaccctcccaccttttccaacccttagggaaccc

SEQ ID NO: 430 - DNA, 49 bp, synthetic construct
  misc_feature 1..49: note = primer
  source 1..49: mol_type = other DNA
tgttcaagaaggaagtgtatcttcatacattccaacccttagggaaccc

SEQ ID NO: 431 - DNA, 46 bp, synthetic construct
  misc_feature 1..46: note = primer
  source 1..46: mol_type = other DNA
atgtgcttttccagactgatccaactgtccaacccttagggaaccc

SEQ ID NO: 432 - DNA, 46 bp, synthetic construct
  misc_feature 1..46: note = primer
  source 1..46: mol_type = other DNA
gtgccagcaagatccaatctagatggaggtggaggtggaggtggag

SEQ ID NO: 433 - DNA, 46 bp, synthetic construct
  misc_feature 1..46: note = primer
  source 1..46: mol_type = other DNA
gtgccagcaagatccaatctagacgtggaggcagaggtggcatggg

SEQ ID NO: 434 - DNA, 48 bp, synthetic construct
  misc_feature 1..48: note = primer
  source 1..48: mol_type = other DNA
gtgccagcaagatccaatctagacgtggtggcttcaataaatttggtg

SEQ ID NO: 435 - DNA, 47 bp, synthetic construct
  misc_feature 1..47: note = primer
  source 1..47: mol_type = other DNA
gtgccagcaagatccaatctagaaccaaggatcacgtcatgactccg

SEQ ID NO: 436 - DNA, 46 bp, synthetic construct
  misc_feature 1..46: note = primer
  source 1..46: mol_type = other DNA
gtgccagcaagatccaatctagaggctgccgtggaatggtttgatg

SEQ ID NO: 437 - DNA, 51 bp, synthetic construct
  misc_feature 1..51: note = primer
  source 1..51: mol_type = other DNA
gtgccagcaagatccaatctagacttctttaagcagtgtggggttgttaag

SEQ ID NO: 438 - DNA, 25 bp, synthetic construct
  misc_feature 1..25: note = primer
  source 1..25: mol_type = other DNA
acatttcatggggctccactaacag

SEQ ID NO: 439 - DNA, 23 bp, synthetic construct
  misc_feature 1..23: note = primer
  source 1..23: mol_type = other DNA
gctgcctgcgtcccaaagaacag

SEQ ID NO: 440 - DNA, 27 bp, synthetic construct
  misc_feature 1..27: note = primer
  source 1..27: mol_type = other DNA
ctacagagacacaacccattgtttatg

SEQ ID NO: 441 - DNA, 27 bp, synthetic construct
  misc_feature 1..27: note = primer
  source 1..27: mol_type = other DNA
gtgggaacgtgaaacatctgatacaag

SEQ ID NO: 442 - DNA, 24 bp, synthetic construct
  misc_feature 1..24: note = primer
  source 1..24: mol_type = other DNA
ggaagattgcccgagagcaaaaag

SEQ ID NO: 443 - DNA, 27 bp, synthetic construct
  misc_feature 1..27: note = primer
  source 1..27: mol_type = other DNA
tgcatattagtggacagcacttagtag

SEQ ID NO: 444 - DNA, 23 bp, synthetic construct
  misc_feature 1..23: note = primer
  source 1..23: mol_type = other DNA
gcagcccaagcttcccatcacag

SEQ ID NO: 445 - DNA, 27 bp, synthetic construct
  misc_feature 1..27: note = primer
  source 1..27: mol_type = other DNA
gaccttccaccaatattcctgaaaatg

SEQ ID NO: 446 - DNA, 27 bp, synthetic construct
  misc_feature 1..27: note = primer
  source 1..27: mol_type = other DNA
ttggcttaacagatgatcaggtttcag

SEQ ID NO: 447 - DNA, 27 bp, synthetic construct
  misc_feature 1..27: note = primer
  source 1..27: mol_type = other DNA
ctcagactcaagcaggtcagattgaag

SEQ ID NO: 448 - DNA, 27 bp, synthetic construct
  misc_feature 1..27: note = primer
  source 1..27: mol_type = other DNA
ctcaacagtatggtattcagtattcag

SEQ ID NO: 449 - DNA, 23 bp, synthetic construct
  misc_feature 1..23: note = primer
  source 1..23: mol_type = other DNA
atatgccctgcgtccaagcccaa

SEQ ID NO: 450 - DNA, 23 bp, synthetic construct
  misc_feature 1..23: note = primer
  source 1..23: mol_type = other DNA
agcccactgcggaagagggcagc

SEQ ID NO: 451 - DNA, 30 bp, synthetic construct
  misc_feature 1..30: note = primer
  source 1..30: mol_type = other DNA
ctgcatcaggagatatgcaaacatatcaga

SEQ ID NO: 452 - DNA, 22 bp, synthetic construct
  misc_feature 1..22: note = primer
  source 1..22: mol_type = other DNA
ttgccattgccccaaatggagc

SEQ ID NO: 453 - DNA, 30 bp, synthetic construct
  misc_feature 1..30: note = primer
  source 1..30: mol_type = other DNA
aaaaattttgaaagacttatcttctgaaga

SEQ ID NO: 454 - DNA, 30 bp, synthetic construct
  misc_feature 1..30: note = primer
  source 1..30: mol_type = other DNA
gtatcatctttatcagaaagtgaggagtcc

SEQ ID NO: 455 - DNA, 23 bp, synthetic construct
  misc_feature 1..23: note = primer
  source 1..23: mol_type = other DNA
ttgccattacccagggaggagca

SEQ ID NO: 456 - DNA, 27 bp, synthetic construct
  misc_feature 1..27: note = primer
  source 1..27: mol_type = other DNA
ctgcctctggagacgtacaaacatacc

SEQ ID NO: 457 - DNA, 28 bp, synthetic construct
  misc_feature 1..28: note = primer
  source 1..28: mol_type = other DNA
atgtgcagcacattaagaggagagacat

SEQ ID NO: 458 - DNA, 23 bp, synthetic construct
  misc_feature 1..23: note = primer
  source 1..23: mol_type = other DNA
gtgcagacccatctggagaaccc

SEQ ID NO: 459 - DNA, 29 bp, synthetic construct
  misc_feature 1..29: note = primer
  source 1..29: mol_type = other DNA
attgatgatgtcattgatgagatcatcag

SEQ ID NO: 460 - DNA, 24 bp, synthetic construct
  misc_feature 1..24: note = primer
  source 1..24: mol_type = other DNA
ctgcctgtgtcagggaatctgctt

SEQ ID NO: 461 - DNA, 26 bp, synthetic construct
  misc_feature 1..26: note = primer
  source 1..26: mol_type = other DNA
ggaaaactacagccaccacacacaag

SEQ ID NO: 462 - DNA, 28 bp, synthetic construct
  misc_feature 1..28: note = primer
  source 1..28: mol_type = other DNA
acacctttcatgaactcaaatctgatgg

SEQ ID NO: 463 - DNA, 29 bp, synthetic construct
  misc_feature 1..29: note = primer
  source 1..29: mol_type = other DNA
ggtcatactgcatcagaaccatgaagaag

SEQ ID NO: 464 - DNA, 24 bp, synthetic construct
  misc_feature 1..24: note = primer
  source 1..24: mol_type = other DNA
ccatgcccattgggagaatagcag

SEQ ID NO: 465 - DNA, 23 bp, synthetic construct
  misc_feature 1..23: note = primer
  source 1..23: mol_type = other DNA
agcaggagcaggagcgggagcgg

SEQ ID NO: 466 - DNA, 23 bp, synthetic construct
  misc_feature 1..23: note = primer
  source 1..23: mol_type = other DNA
cgccggagttgcataagggagat

SEQ ID NO: 467 - DNA, 24 bp, synthetic construct
  misc_feature 1..24: note = primer
  source 1..24: mol_type = other DNA
ggttccatgatgggaagtgacatg

SEQ ID NO: 468 - DNA, 28 bp, synthetic construct
  misc_feature 1..28: note = primer
  source 1..28: mol_type = other DNA
tggccaatgtgatctggaacttattaat

SEQ ID NO: 469 - DNA, 23 bp, synthetic construct
  misc_feature 1..23: note = primer
  source 1..23: mol_type = other DNA
tcaagggaaccttccctgatgcg

SEQ ID NO: 470 - DNA, 27 bp, synthetic construct
  misc_feature 1..27: note = primer
  source 1..27: mol_type = other DNA
cagtgatctggcctcagacaactactg

SEQ ID NO: 471 - DNA, 29 bp, synthetic construct
  misc_feature 1..29: note = primer
  source 1..29: mol_type = other DNA
gcataagctggaagtcacaccagtagtag

SEQ ID NO: 472 - DNA, 27 bp, synthetic construct
  misc_feature 1..27: note = primer
  source 1..27: mol_type = other DNA
aggttgaaattgggtcttcaaaaccag

SEQ ID NO: 473 - DNA, 23 bp, synthetic construct
  misc_feature 1..23: note = primer
  source 1..23: mol_type = other DNA
tcaggatgggaaaattgcaccag

SEQ ID NO: 474 - DNA, 35 bp, synthetic construct
  misc_feature 1..35: note = primer
  source 1..35: mol_type = other DNA
gagaatatataaaaaactggaggccaagatacttc

SEQ ID NO: 475 - DNA, 30 bp, synthetic construct; identical to SEQ ID NO: 77
  misc_feature 1..30: note = primer
  source 1..30: mol_type = other DNA

SEQ ID NO: 476 - DNA, 30 bp, synthetic construct; identical to SEQ ID NO: 78
  misc_feature 1..30: note = primer
  source 1..30: mol_type = other DNA

SEQ ID NO: 477 - DNA, 30 bp, synthetic construct; identical to SEQ ID NO: 451
  misc_feature 1..30: note = primer
  source 1..30: mol_type = other DNA

SEQ ID NO: 478 - DNA, 22 bp, synthetic construct
  misc_feature 1..22: note = primer
  source 1..22: mol_type = other DNA
ttgccattgccccaaatggagc

SEQ ID NO: 479 - DNA, 30 bp, synthetic construct; identical to SEQ ID NO: 453
  misc_feature 1..30: note = primer
  source 1..30: mol_type = other DNA

SEQ ID NO: 480 - DNA, 30 bp, synthetic construct; identical to SEQ ID NO: 454
  misc_feature 1..30: note = primer
  source 1..30: mol_type = other DNA

SEQ ID NO: 481 - DNA, 48 bp, synthetic construct
  misc_feature 1..48: note = primer
  source 1..48: mol_type = other DNA
gtgccagcaagatccaatctagaacatttcatggggctccactaacag

SEQ ID NO: 482 - DNA, 46 bp, synthetic construct
  misc_feature 1..46: note = primer
  source 1..46: mol_type = other DNA
gtgccagcaagatccaatctagagctgcctgcgtcccaaagaacag

SEQ ID NO: 483 - DNA, 50 bp, synthetic construct
  misc_feature 1..50: note = primer
  source 1..50: mol_type = other DNA
gtgccagcaagatccaatctagactacagagacacaacccattgtttatg

SEQ ID NO: 484 - DNA, 50 bp, synthetic construct
  misc_feature 1..50: note = primer
  source 1..50: mol_type = other DNA
gtgccagcaagatccaatctagagtgggaacgtgaaacatctgatacaag

SEQ ID NO: 485 - DNA, 47 bp, synthetic construct
  misc_feature 1..47: note = primer
  source 1..47: mol_type = other DNA
gtgccagcaagatccaatctagaggaagattgcccgagagcaaaaag

SEQ ID NO: 486 - DNA, 50 bp, synthetic construct
  misc_feature 1..50: note = primer
  source 1..50: mol_type = other DNA
gtgccagcaagatccaatctagatgcatattagtggacagcacttagtag

SEQ ID NO: 487 - DNA, 46 bp, synthetic construct
  misc_feature 1..46: note = primer
  source 1..46: mol_type = other DNA
gtgccagcaagatccaatctagagcagcccaagcttcccatcacag

SEQ ID NO: 488 - DNA, 50 bp, synthetic construct
  misc_feature 1..50: note = primer
  source 1..50: mol_type = other DNA
gtgccagcaagatccaatctagagaccttccaccaatattcctgaaaatg

SEQ ID NO: 489 - DNA, 50 bp, synthetic construct
  misc_feature 1..50: note = primer
  source 1..50: mol_type = other DNA
gtgccagcaagatccaatctagattggcttaacagatgatcaggtttcag

SEQ ID NO: 490 - DNA, 50 bp, synthetic construct
  misc_feature 1..50: note = primer
  source 1..50: mol_type = other DNA
gtgccagcaagatccaatctagactcagactcaagcaggtcagattgaag

SEQ ID NO: 491 - DNA, 50 bp, synthetic construct
  misc_feature 1..50: note = primer
  source 1..50: mol_type = other DNA
gtgccagcaagatccaatctagactcaacagtatggtattcagtattcag

SEQ ID NO: 492 - DNA, 42 bp, synthetic construct
  misc_feature 1..42: note = primer
  source 1..42: mol_type = other DNA
atatgccctgcgtccaagcccaatccaacccttagggaaccc

SEQ ID NO: 493 - DNA, 42 bp, synthetic construct
  misc_feature 1..42: note = primer
  source 1..42: mol_type = other DNA
agcccactgcggaagagggcagctccaacccttagggaaccc

SEQ ID NO: 494 - DNA, 49 bp, synthetic construct
  misc_feature 1..49: note = primer
  source 1..49: mol_type = other DNA
ctgcatcaggagatatgcaaacatatcagatccaacccttagggaaccc

SEQ ID NO: 495 - DNA, 41 bp, synthetic construct
  misc_feature 1..41: note = primer
  source 1..41: mol_type = other DNA
ttgccattgccccaaatggagctccaacccttagggaaccc

SEQ ID NO: 496 - DNA, 49 bp, synthetic construct
  misc_feature 1..49: note = primer
  source 1..49: mol_type = other DNA
aaaaattttgaaagacttatcttctgaagatccaacccttagggaaccc

SEQ ID NO: 497 - DNA, 49 bp, synthetic construct
  misc_feature 1..49: note = primer
  source 1..49: mol_type = other DNA
gtatcatctttatcagaaagtgaggagtcctccaacccttagggaaccc

SEQ ID NO: 498 - DNA, 42 bp, synthetic construct
  misc_feature 1..42: note = primer
  source 1..42: mol_type = other DNA
ttgccattacccagggaggagcatccaacccttagggaaccc

SEQ ID NO: 499 - DNA, 46 bp, synthetic construct
  misc_feature 1..46: note = primer
  source 1..46: mol_type = other DNA
ctgcctctggagacgtacaaacatacctccaacccttagggaaccc

SEQ ID NO: 500 - DNA, 47 bp, synthetic construct
  misc_feature 1..47: note = primer
  source 1..47: mol_type = other DNA
atgtgcagcacattaagaggagagacattccaacccttagggaaccc

SEQ ID NO: 501 - DNA, 42 bp, synthetic construct
  misc_feature 1..42: note = primer
  source 1..42: mol_type = other DNA
gtgcagacccatctggagaaccctccaacccttagggaaccc

SEQ ID NO: 502 - DNA, 48 bp, synthetic construct
  misc_feature 1..48: note = primer
  source 1..48: mol_type = other DNA
attgatgatgtcattgatgagatcatcagtccaacccttagggaaccc

SEQ ID NO: 503 - DNA, 43 bp, synthetic construct
  misc_feature 1..43: note = primer
  source 1..43: mol_type = other DNA
ctgcctgtgtcagggaatctgctttccaacccttagggaaccc

SEQ ID NO: 504 - DNA, 49 bp, synthetic construct
  misc_feature 1..49: note = primer
  source 1..49: mol_type = other DNA
gtgccagcaagatccaatctagaggaaaactacagccaccacacacaag

SEQ ID NO: 505 - DNA, 51 bp, synthetic construct
  misc_feature 1..51: note = primer
  source 1..51: mol_type = other DNA
gtgccagcaagatccaatctagaacacctttcatgaactcaaatctgatgg

SEQ ID NO: 506 - DNA, 52 bp, synthetic construct; identical to SEQ ID NO: 56
  misc_feature 1..52: note = primer
  source 1..52: mol_type = other DNA

SEQ ID NO: 507 - DNA, 47 bp, synthetic construct; identical to SEQ ID NO: 57
  misc_feature 1..47: note = primer
  source 1..47: mol_type = other DNA

SEQ ID NO: 508 - DNA, 46 bp, synthetic construct
  misc_feature 1..46: note = primer
  source 1..46: mol_type = other DNA
gtgccagcaagatccaatctagaagcaggagcaggagcgggagcgg

SEQ ID NO: 509 - DNA, 46 bp, synthetic construct
  misc_feature 1..46: note = primer
  source 1..46: mol_type = other DNA
gtgccagcaagatccaatctagacgccggagttgcataagggagat

SEQ ID NO: 510 - DNA, 47 bp, synthetic construct
  misc_feature 1..47: note = primer
  source 1..47: mol_type = other DNA
gtgccagcaagatccaatctagaggttccatgatgggaagtgacatg

SEQ ID NO: 511 - DNA, 51 bp, synthetic construct
  misc_feature 1..51: note = primer
  source 1..51: mol_type = other DNA
gtgccagcaagatccaatctagatggccaatgtgatctggaacttattaat

SEQ ID NO: 512 - DNA, 46 bp, synthetic construct
  misc_feature 1..46: note = primer
  source 1..46: mol_type = other DNA
gtgccagcaagatccaatctagatcaagggaaccttccctgatgcg

SEQ ID NO: 513 - DNA, 50 bp, synthetic construct
  misc_feature 1..50: note = primer
  source 1..50: mol_type = other DNA
gtgccagcaagatccaatctagacagtgatctggcctcagacaactactg

SEQ ID NO: 514 - DNA, 48 bp, synthetic construct
  misc_feature 1..48: note = primer
  source 1..48: mol_type = other DNA
gcataagctggaagtcacaccagtagtagtccaacccttagggaaccc

SEQ ID NO: 515 - DNA, 50 bp, synthetic construct
  misc_feature 1..50: note = primer
  source 1..50: mol_type = other DNA
gtgccagcaagatccaatctagaaggttgaaattgggtcttcaaaaccag

SEQ ID NO: 516 - DNA, 46 bp, synthetic construct
  misc_feature 1..46: note = primer
  source 1..46: mol_type = other DNA
gtgccagcaagatccaatctagatcaggatgggaaaattgcaccag

SEQ ID NO: 517 - DNA, 54 bp, synthetic construct
  misc_feature 1..54: note = primer
  source 1..54: mol_type = other DNA
gagaatatataaaaaactggaggccaagatacttctccaacccttagggaaccc

SEQ ID NO: 518 - DNA, 53 bp, synthetic construct; identical to SEQ ID NO: 231
  misc_feature 1..53: note = primer
  source 1..53: mol_type = other DNA

SEQ ID NO: 519 - DNA, 53 bp, synthetic construct; identical to SEQ ID NO: 232
  misc_feature 1..53: note = primer
  source 1..53: mol_type = other DNA

SEQ ID NO: 520 - DNA, 49 bp, synthetic construct; identical to SEQ ID NO: 494
  misc_feature 1..49: note = primer
  source 1..49: mol_type = other DNA

SEQ ID NO: 521 - DNA, 41 bp, synthetic construct; identical to SEQ ID NO: 495
  misc_feature 1..41: note = primer
  source 1..41: mol_type = other DNA

SEQ ID NO: 522 - DNA, 49 bp, synthetic construct; identical to SEQ ID NO: 496
  misc_feature 1..49: note = primer
  source 1..49: mol_type = other DNA

SEQ ID NO: 523 - DNA, 49 bp, synthetic construct; identical to SEQ ID NO: 497
  misc_feature 1..49: note = primer
  source 1..49: mol_type = other DNA

SEQ ID NO: 524 - DNA, 26 bp, synthetic construct
  misc_feature 1..26: note = primer
  source 1..26: mol_type = other DNA
gttattccaggatctttggagacccg

SEQ ID NO: 525 - DNA, 28 bp, synthetic construct
  misc_feature 1..28: note = primer
  source 1..28: mol_type = other DNA
gaagccttatcagttgtgagtgaggacc

SEQ ID NO: 526 - DNA, 24 bp, synthetic construct
  misc_feature 1..24: note = primer
  source 1..24: mol_type = other DNA
atttaccatatgagccccccagga

SEQ ID NO: 527 - DNA, 25 bp, synthetic construct
  misc_feature 1..25: note = primer
  source 1..25: mol_type = other DNA
ctgctcaaccatctccttccacagt

SEQ ID NO: 528 - DNA, 30 bp, synthetic construct; identical to SEQ ID NO: 385
  misc_feature 1..30: note = primer
  source 1..30: mol_type = other DNA

SEQ ID NO: 529 - DNA, 22 bp, synthetic construct
  misc_feature 1..22: note = primer
  source 1..22: mol_type = other DNA
gcagtggccagatccagctttg

SEQ ID NO: 530 - DNA, 22 bp, synthetic construct
  misc_feature 1..22: note = primer
  source 1..22: mol_type = other DNA
agcgccgcctggagcgcggcag

SEQ ID NO: 531 - DNA, 27 bp, synthetic construct
  misc_feature 1..27: note = primer
  source 1..27: mol_type = other DNA
gataacagcaagatggctttgaactca

SEQ ID NO: 532 - DNA, 23 bp, synthetic construct
  misc_feature 1..23: note = primer
  source 1..23: mol_type = other DNA
tgtaccgccggaagcaccaggag

SEQ ID NO: 533 - DNA, 23 bp, synthetic construct
  misc_feature 1..23: note = primer
  source 1..23: mol_type = other DNA
ggctggaaacatttccgaccctg

SEQ ID NO: 534 - DNA, 27 bp, synthetic construct
  misc_feature 1..27: note = primer
  source 1..27: mol_type = other DNA
tggaaaagacaattgatgacctggaag

SEQ ID NO: 535 - DNA, 27 bp, synthetic construct
  misc_feature 1..27: note = primer
  source 1..27: mol_type = other DNA
cagtgaaaaaatcagtctcaagtaaag

SEQ ID NO: 536 - DNA, 27 bp, synthetic construct
  misc_feature 1..27: note = primer
  source 1..27: mol_type = other DNA
agcataaagatgtcatcatcaaccaag

SEQ ID NO: 537 - DNA, 25 bp, synthetic construct
  misc_feature 1..25: note = primer
  source 1..25: mol_type = other DNA
cccacacctgggaaaggacctaaag

SEQ ID NO: 538 - DNA, 27 bp, synthetic construct
  misc_feature 1..27: note = primer
  source 1..27: mol_type = other DNA
gatctgaatcctgaaagagaaatagag

SEQ ID NO: 539 - DNA, 23 bp, synthetic construct
  misc_feature 1..23: note = primer
  source 1..23: mol_type = other DNA
gccataggaacgcactcaggcag

SEQ ID NO: 540 - DNA, 27 bp, synthetic construct
  misc_feature 1..27: note = primer
  source 1..27: mol_type = other DNA
agctctctgtgatgcgctactcaatag

SEQ ID NO: 541 - DNA, 27 bp, synthetic construct
  misc_feature 1..27: note = primer
  source 1..27: mol_type = other DNA
actcgggagactatgaaatattgtact

SEQ ID NO: 542 - DNA, 25 bp, synthetic construct
  misc_feature 1..25: note = primer
  source 1..25: mol_type = other DNA
ctggagtcccaaataaaccaggcat

SEQ ID NO: 543 - DNA, 30 bp, synthetic construct
  misc_feature 1..30: note = primer
  source 1..30: mol_type = other DNA
atgatttttggataccagaaacaagtttca

SEQ ID NO: 544 - DNA, 30 bp, synthetic construct
  misc_feature 1..30: note = primer
  source 1..30: mol_type = other DNA
tctggcatagaagattaaagaatcaaaaaa

SEQ ID NO: 545 - DNA, 24 bp, synthetic construct
  misc_feature 1..24: note = primer
  source 1..24: mol_type = other DNA
agctgtctggctctggagatctgg

SEQ ID NO: 546 - DNA, 23 bp, synthetic construct
  misc_feature 1..23: note = primer
  source 1..23: mol_type = other DNA
tgagagaacggaggtcctggcag

SEQ ID NO: 547 - DNA, 27 bp, synthetic construct
  misc_feature 1..27: note = primer
  source 1..27: mol_type = other DNA
acataaccattagcagagaggctcagg

SEQ ID NO: 548 - DNA, 23 bp, synthetic construct
  misc_feature 1..23: note = primer
  source 1..23: mol_type = other DNA
gcccactgacgctccaccgaaag

SEQ ID NO: 549 - DNA, 26 bp, synthetic construct
  misc_feature 1..26: note = primer
  source 1..26: mol_type = other DNA
ggagaagacaaagaaggcagagagag

SEQ ID NO: 550 - DNA, 27 bp, synthetic construct
  misc_feature 1..27: note = primer
  source 1..27: mol_type = other DNA
ttttcttaccacaacatgacagtagtg

SEQ ID NO: 551 - DNA, 23 bp, synthetic construct
  misc_feature 1..23: note = primer
  source 1..23: mol_type = other DNA
atccactgtgcgacgagctgtgc

SEQ ID NO: 552 - DNA, 24 bp, synthetic construct
  misc_feature 1..24: note = primer
  source 1..24: mol_type = other DNA
gaggatccaaagtgggaattccct

SEQ ID NO: 553 - DNA, 27 bp, synthetic construct
  misc_feature 1..27: note = primer
  source 1..27: mol_type = other DNA
attgctgtgggaaataatgatgtaaag

SEQ ID NO: 554 - DNA, 27 bp, synthetic construct
  misc_feature 1..27: note = primer
  source 1..27: mol_type = other DNA
gcagcatgtcagcttcgtatctctcaa

SEQ ID NO: 555 - DNA, 27 bp, synthetic construct
  misc_feature 1..27: note = primer
  source 1..27: mol_type = other DNA
aagaactagtccagcttcgagcacaag

SEQ ID NO: 556 - DNA, 27 bp, synthetic construct
  misc_feature 1..27: note = primer
  source 1..27: mol_type = other DNA
caggacctggctacaagagttaaaaag

SEQ ID NO: 557 - DNA, 27 bp, synthetic construct
  misc_feature 1..27: note = primer
  source 1..27: mol_type = other DNA
gaacagctcactaaagtgcacaaacag

SEQ ID NO: 558 - DNA, 25 bp, synthetic construct
  misc_feature 1..25: note = primer
  source 1..25: mol_type = other DNA
agaagagggcattctgcacagattg

SEQ ID NO: 559 - DNA, 23 bp, synthetic construct
  misc_feature 1..23: note = primer
  source 1..23: mol_type = other DNA
tgcgcaaagccagcgtgaccatc

SEQ ID NO: 560 - DNA, 45 bp, synthetic construct; identical to SEQ ID NO: 413
  misc_feature 1..45: note = primer
  source 1..45: mol_type = other DNA

SEQ ID NO: 561 - DNA, 47 bp, synthetic construct; identical to SEQ ID NO: 414
  misc_feature 1..47: note = primer
  source 1..47: mol_type = other DNA

SEQ ID NO: 562 - DNA, 43 bp, synthetic construct; identical to SEQ ID NO: 415
  misc_feature 1..43: note = primer
  source 1..43: mol_type = other DNA

SEQ ID NO: 563 - DNA, 44 bp, synthetic construct; identical to SEQ ID NO: 416
  misc_feature 1..44: note = primer
  source 1..44: mol_type = other DNA

SEQ ID NO: 564 - DNA, 49 bp, synthetic construct; identical to SEQ ID NO: 417
  misc_feature 1..49: note = primer
  source 1..49: mol_type = other DNA

SEQ ID NO: 565 - DNA, 41 bp, synthetic construct; identical to SEQ ID NO: 418
  misc_feature 1..41: note = primer
  source 1..41: mol_type = other DNA

SEQ ID NO: 566 - DNA, 45 bp, synthetic construct
  misc_feature 1..45: note = primer
  source 1..45: mol_type = other DNA
gtgccagcaagatccaatctagaagcgccgcctggagcgcggcag

SEQ ID NO: 567 - DNA, 50 bp, synthetic construct
  misc_feature 1..50: note = primer
  source 1..50: mol_type = other DNA
gtgccagcaagatccaatctagagataacagcaagatggctttgaactca

SEQ ID NO: 568 - DNA, 42 bp, synthetic construct
  misc_feature 1..42: note = primer
  source 1..42: mol_type = other DNA
tgtaccgccggaagcaccaggagtccaacccttagggaaccc

SEQ ID NO: 569 - DNA, 46 bp, synthetic construct
  misc_feature 1..46: note = primer
  source 1..46: mol_type = other DNA
gtgccagcaagatccaatctagaggctggaaacatttccgaccctg

SEQ ID NO: 570 - DNA, 50 bp, synthetic construct
  misc_feature 1..50: note = primer
  source 1..50: mol_type = other DNA
gtgccagcaagatccaatctagatggaaaagacaattgatgacctggaag

SEQ ID NO: 571 - DNA, 50 bp, synthetic construct
  misc_feature 1..50: note = primer
  source 1..50: mol_type = other DNA
gtgccagcaagatccaatctagacagtgaaaaaatcagtctcaagtaaag

SEQ ID NO: 572 - DNA, 50 bp, synthetic construct
  misc_feature 1..50: note = primer
  source 1..50: mol_type = other DNA
gtgccagcaagatccaatctagaagcataaagatgtcatcatcaaccaag

SEQ ID NO: 573 - DNA, 48 bp, synthetic construct
  misc_feature 1..48: note = primer
  source 1..48: mol_type = other DNA
gtgccagcaagatccaatctagacccacacctgggaaaggacctaaag

SEQ ID NO: 574 - DNA, 50 bp, synthetic construct
  misc_feature 1..50: note = primer
  source 1..50: mol_type = other DNA
gtgccagcaagatccaatctagagatctgaatcctgaaagagaaatagag

SEQ ID NO: 575 - DNA, 46 bp, synthetic construct
  misc_feature 1..46: note = primer
  source 1..46: mol_type = other DNA
gtgccagcaagatccaatctagagccataggaacgcactcaggcag

SEQ ID NO: 576 - DNA, 50 bp, synthetic construct
  misc_feature 1..50: note = primer
  source 1..50: mol_type = other DNA
gtgccagcaagatccaatctagaagctctctgtgatgcgctactcaatag

SEQ ID NO: 577 - DNA, 50 bp, synthetic construct
  misc_feature 1..50: note = primer
  source 1..50: mol_type = other DNA
gtgccagcaagatccaatctagaactcgggagactatgaaatattgtact

SEQ ID NO: 578 - DNA, 44 bp, synthetic construct
  misc_feature 1..44: note = primer
  source 1..44: mol_type = other DNA
ctggagtcccaaataaaccaggcattccaacccttagggaaccc

SEQ ID NO: 579 - DNA, 49 bp, synthetic construct
  misc_feature 1..49: note = primer
  source 1..49: mol_type = other DNA
atgatttttggataccagaaacaagtttcatccaacccttagggaaccc

SEQ ID NO: 580 - DNA, 49 bp, synthetic construct
  misc_feature 1..49: note = primer
  source 1..49: mol_type = other DNA
tctggcatagaagattaaagaatcaaaaaatccaacccttagggaaccc

SEQ ID NO: 581 - DNA, 46 bp, synthetic construct
  misc_feature 1..46: note = primer
  source 1..46: mol_type = other DNA
gtgccagcaagatccaatctagaagctgtctggctctggagatctg

SEQ ID NO: 582 - DNA, 46 bp, synthetic construct
  misc_feature 1..46: note = primer
  source 1..46: mol_type = other DNA
gtgccagcaagatccaatctagatgagagaacggaggtcctggcag

SEQ ID NO: 583 - DNA, 50 bp, synthetic construct
  misc_feature 1..50: note = primer
  source 1..50: mol_type = other DNA
gtgccagcaagatccaatctagaacataaccattagcagagaggctcagg

SEQ ID NO: 584 - DNA, 46 bp, synthetic construct
  misc_feature 1..46: note = primer
  source 1..46: mol_type = other DNA
gtgccagcaagatccaatctagagcccactgacgctccaccgaaag

SEQ ID NO: 585 - DNA, 49 bp, synthetic construct
  misc_feature 1..49: note = primer
  source 1..49: mol_type = other DNA
gtgccagcaagatccaatctagaggagaagacaaagaaggcagagagag

SEQ ID NO: 586 - DNA, 50 bp, synthetic construct
  misc_feature 1..50: note = primer
  source 1..50: mol_type = other DNA
gtgccagcaagatccaatctagattttcttaccacaacatgacagtagtg

SEQ ID NO: 587 - DNA, 42 bp, synthetic construct
  misc_feature 1..42: note = primer
  source 1..42: mol_type = other DNA
atccactgtgcgacgagctgtgctccaacccttagggaaccc

SEQ ID NO: 588 - DNA, 43 bp, synthetic construct
  misc_feature 1..43: note = primer
  source 1..43: mol_type = other DNA
gaggatccaaagtgggaattcccttccaacccttagggaaccc

SEQ ID NO: 589 - DNA, 50 bp, synthetic construct
  misc_feature 1..50: note = primer
  source 1..50: mol_type = other DNA
gtgccagcaagatccaatctagaattgctgtgggaaataatgatgtaaag

SEQ ID NO: 590 - DNA, 50 bp, synthetic construct
  misc_feature 1..50: note = primer
  source 1..50: mol_type = other DNA
gtgccagcaagatccaatctagagcagcatgtcagcttcgtatctctcaa

SEQ ID NO: 591 - DNA, 50 bp, synthetic construct
  misc_feature 1..50: note = primer
  source 1..50: mol_type = other DNA
gtgccagcaagatccaatctagaaagaactagtccagcttcgagcacaag

SEQ ID NO: 592 - DNA, 50 bp, synthetic construct
  misc_feature 1..50: note = primer
  source 1..50: mol_type = other DNA
gtgccagcaagatccaatctagacaggacctggctacaagagttaaaaag

SEQ ID NO: 593 - DNA, 50 bp, synthetic construct
  misc_feature 1..50: note = primer
  source 1..50: mol_type = other DNA
gtgccagcaagatccaatctagagaacagctcactaaagtgcacaaacag

SEQ ID NO: 594 - DNA, 48 bp, synthetic construct
  misc_feature 1..48: note = primer
  source 1..48: mol_type = other DNA
gtgccagcaagatccaatctagaagaagagggcattctgcacagattg

SEQ ID NO: 595 - DNA, 46 bp, synthetic construct
  misc_feature 1..46: note = primer
  source 1..46: mol_type = other DNA
gtgccagcaagatccaatctagatgcgcaaagccagcgtgaccatc

SEQ ID NO: 596 - DNA, 21 bp, synthetic construct
  misc_feature 1..21: note = primer forward
  source 1..21: mol_type = other DNA
cgcaaatgggcggtaggcgtg

SEQ ID NO: 597 - DNA, 19 bp, synthetic construct
  misc_feature 1..19: note = primer reverse
  source 1..19: mol_type = other DNA
cgccatccacgctgttttg

SEQ ID NO: 598 - DNA, 21 bp, synthetic construct
  misc_feature 1..21: note = primer forward
  source 1..21: mol_type = other DNA
ggctaactagagaacccactg

SEQ ID NO: 599 - DNA, 20 bp, synthetic construct
  misc_feature 1..20: note = primer reverse
  source 1..20: mol_type = other DNA
ggcaactagaaggcacagtc

SEQ ID NO: 600 - DNA, 20 bp, synthetic construct
  misc_feature 1..20: note = primer forward
  source 1..20: mol_type = other DNA
agagctcgtttagtgaaccg

SEQ ID NO: 601 - DNA, 20 bp, synthetic construct
  misc_feature 1..20: note = primer reverse
  source 1..20: mol_type = other DNA
gtggtttgtccaaactcatc

SEQ ID NO: 602 - DNA, 19 bp, synthetic construct
  misc_feature 1..19: note = primer forward
  source 1..19: mol_type = other DNA
gatcactctcggcatggac

SEQ ID NO: 603 - DNA, 23 bp, synthetic construct
  misc_feature 1..23: note = primer reverse
  source 1..23: mol_type = other DNA
cattttatgtttcaggttcaggg

SEQ ID NO: 604 - DNA, 18 bp, synthetic construct
  misc_feature 1..18: note = primer forward
  source 1..18: mol_type = other DNA
gtcgtaacaactccgccc

SEQ ID NO: 605 - DNA, 19 bp, synthetic construct
  misc_feature 1..19: note = primer reverse
  source 1..19: mol_type = other DNA
gtccagctcgaccaggatg

SEQ ID NO: 606 - DNA, 19 bp, synthetic construct
  misc_feature 1..19: note = primer forward
  source 1..19: mol_type = other DNA
atagcatggcctttgcagg

SEQ ID NO: 607 - DNA, 19 bp, synthetic construct
  misc_feature 1..19: note = primer reverse
  source 1..19: mol_type = other DNA
gagctgcatgtgtcagagg

SEQ ID NO: 608 - DNA, 22 bp, synthetic construct
  misc_feature 1..22: note = primer forward
  source 1..22: mol_type = other DNA
gtatcttatggtactgtaactg

SEQ ID NO: 609 - DNA, 22 bp, synthetic construct
  misc_feature 1..22: note = primer reverse
  source 1..22: mol_type = other DNA
ctttatgtttttggcgtcttcc

SEQ ID NO: 610 - DNA, 20 bp, synthetic construct
  misc_feature 1..20: note = primer forward
  source 1..20: mol_type = other DNA
ggtctatataagcagagctg

SEQ ID NO: 611 - DNA, 23 bp, synthetic construct
  misc_feature 1..23: note = primer reverse
  source 1..23: mol_type = other DNA
gtggtatggctgattatgatcag

SEQ ID NO: 612 - DNA, 26 bp, synthetic construct
  misc_feature 1..26: note = AF6 exon 2
  source 1..26: mol_type = other DNA
ctcatcactccatggaactccaaatc

SEQ ID NO: 613 - DNA, 24 bp, synthetic construct
  misc_feature 1..24: note = MLL exon 9
  source 1..24: mol_type = other DNA
cttttcttttggtttttgttttac

SEQ ID NO: 614 - DNA, 23 bp, synthetic construct
  misc_feature 1..23: note = AF1Q exon 2
  source 1..23: mol_type = other DNA
ctcacagggtccctcatagcttc

SEQ ID NO: 615 - DNA, 13 bp, synthetic construct
  misc_feature 1..13: note = MLL exon 10
  source 1..13: mol_type = other DNA
cttaaagtccact

SEQ ID NO: 616 - DNA, 23 bp, synthetic construct
  misc_feature 1..23: note = primer
  source 1..23: mol_type = other DNA
ctctacgtctcctccgagagccg

SEQ ID NO: 617 - DNA, 23 bp, synthetic construct
  misc_feature 1..23: note = primer
  source 1..23: mol_type = other DNA
gtctcctcctcggcttcctcttc

SEQ ID NO: 618 - DNA, 29 bp, synthetic construct
  misc_feature 1..29: note = primer
  source 1..29: mol_type = other DNA
ataatctcagtgataccttgaagaagctg

SEQ ID NO: 619 - DNA, 28 bp, synthetic construct
  misc_feature 1..28: note = primer
  source 1..28: mol_type = other DNA
aaatgacatcagatgtaccatcactggg

SEQ ID NO: 620 - DNA, 29 bp, synthetic construct
  misc_feature 1..29: note = primer
  source 1..29: mol_type = other DNA
tcgcttcatggagatataagtagcctgaa

SEQ ID NO: 621 - DNA, 27 bp, synthetic construct
  misc_feature 1..27: note = primer
  source 1..27: mol_type = other DNA
gaaattcggcgccttcatcagtatgtg

SEQ ID NO: 622 - DNA, 30 bp, synthetic construct
  misc_feature 1..30: note = primer
  source 1..30: mol_type = other DNA
atcgtactgagaagcactccacaatgccag

SEQ ID NO: 623 - DNA, 33 bp, synthetic construct
  misc_feature 1..33: note = primer
  source 1..33: mol_type = other DNA
caggaacgaatttcatatacacctccagagagc

SEQ ID NO: 624 - DNA, 26 bp, synthetic construct
  misc_feature 1..26: note = primer
  source 1..26: mol_type = other DNA
gcttgcagccaatttactggagcagg

SEQ ID NO: 625 - DNA, 33 bp, synthetic construct
  misc_feature 1..33: note = primer
  source 1..33: mol_type = other DNA
atcttagacgaattttacaatgtgaagttctgc

SEQ ID NO: 626 - DNA, 24 bp, synthetic construct
  misc_feature 1..24: note = primer
  source 1..24: mol_type = other DNA
gtgtctgctgactccagtgcatcc

SEQ ID NO: 627 - DNA, 28 bp, synthetic construct
  misc_feature 1..28: note = primer
  source 1..28: mol_type = other DNA
attgtcagtaaacgggcaggtactcagt

SEQ ID NO: 628 - DNA, 27 bp, synthetic construct
  misc_feature 1..27: note = primer
  source 1..27: mol_type = other DNA
gatgacagcatggaagagaaaccacta

SEQ ID NO: 629 - DNA, 23 bp, synthetic construct
  misc_feature 1..23: note = primer
  source 1..23: mol_type = other DNA
tgagtccattcttgcacaccgag

SEQ ID NO: 630 - DNA, 27 bp, synthetic construct
  misc_feature 1..27: note = primer
  source 1..27: mol_type = other DNA
attttggctctctatttgacttggagc

SEQ ID NO: 631 - DNA, 24 bp, synthetic construct
  misc_feature 1..24: note = primer
  source 1..24: mol_type = other DNA
atgggaatgatgaacaaccccaat

SEQ ID NO: 632 - DNA, 23 bp, synthetic construct
  misc_feature 1..23: note = primer
  source 1..23: mol_type = other DNA
ccttgccctttaaggtggtggtg

SEQ ID NO: 633 - DNA, 28 bp, synthetic construct
  misc_feature 1..28: note = primer
  source 1..28: mol_type = other DNA
ctgccttatgactcaagatgggagtttc

SEQ ID NO: 634 - DNA, 22 bp, synthetic construct
  misc_feature 1..22: note = primer
  source 1..22: mol_type = other DNA
gggccccgagaacctcgaaatc

SEQ ID NO: 635 - DNA, 22 bp, synthetic construct
  misc_feature 1..22: note = primer
  source 1..22: mol_type = other DNA
gggcgacctcttccagaagctg

SEQ ID NO: 636 - DNA, 27 bp, synthetic construct
  misc_feature 1..27: note = primer
  source 1..27: mol_type = other DNA
atccaacgaccaagaactctctggaaa

SEQ ID NO: 637 - DNA, 28 bp, synthetic construct
  misc_feature 1..28: note = primer
  source 1..28: mol_type = other DNA
gccagttatttgcctcagtaaaagacag

SEQ ID NO: 638 - DNA, 30 bp, synthetic construct
  misc_feature 1..30: note = primer
  source 1..30: mol_type = other DNA
cagtatgagtacacggagctcaagaaacag

SEQ ID NO: 639 - DNA, 28 bp, synthetic construct
  misc_feature 1..28: note = primer
  source 1..28: mol_type = other DNA
cacaatgaaacagatttgcaaaaaggaa

SEQ ID NO: 640 - DNA, 20 bp, synthetic construct
  misc_feature 1..20: note = primer
  source 1..20: mol_type = other DNA
ccgcctgcctgcgcacctgc

SEQ ID NO: 641 - DNA, 22 bp, synthetic construct
  misc_feature 1..22: note = primer
  source 1..22: mol_type = other DNA
cgggcagaaggctggtgacaag

SEQ ID NO: 642 - DNA, 20 bp, synthetic construct
  misc_feature 1..20: note = primer
  source 1..20: mol_type = other DNA
aagggccaaccactggggaa

SEQ ID NO: 643 - DNA, 30 bp, synthetic construct
  misc_feature 1..30: note = primer
  source 1..30: mol_type = other DNA
aaaacaagtgcacagacaacaccaagtaag

SEQ ID NO: 644 - DNA, 26 bp, synthetic construct
  misc_feature 1..26: note = primer
  source 1..26: mol_type = other DNA
gggcaaatgagaacagcaacatacag

SEQ ID NO: 645 - DNA, 26 bp, synthetic construct
  misc_feature 1..26: note = primer
  source 1..26: mol_type = other DNA
gcacacctctcaatgcagctttacag

SEQ ID NO: 646 - DNA, 29 bp, synthetic construct
  misc_feature 1..29: note = primer
  source 1..29: mol_type = other DNA
tgaggacaagttctacagccacaagaaaa

SEQ ID NO: 647 - DNA, 24 bp, synthetic construct
  misc_feature 1..24: note = primer
  source 1..24: mol_type = other DNA
agaaccccaacagcaaagaaggct

SEQ ID NO: 648 - DNA, 25 bp, synthetic construct
  misc_feature 1..25: note = primer
  source 1..25: mol_type = other DNA
cctaagatgcccgacttcaactgct

SEQ ID NO: 649 - DNA, 24 bp, synthetic construct
  misc_feature 1..24: note = primer
  source 1..24: mol_type = other DNA
aatgctggacttccgtagtgacca

SEQ ID NO: 650 - DNA, 30 bp, synthetic construct
  misc_feature 1..30: note = primer
  source 1..30: mol_type = other DNA
cagtgccaggaaagagaattagagatcagt

SEQ ID NO: 651 - DNA, 23 bp, synthetic construct
  misc_feature 1..23: note = primer
  source 1..23: mol_type = other DNA
caaagcccacgctgaagcgaaag

SEQ ID NO: 652 - DNA, 26 bp, synthetic construct
  misc_feature 1..26: note = primer
  source 1..26: mol_type = other DNA
agctaaaaggacagcaggtgctacca

SEQ ID NO: 653 - DNA, 28 bp, synthetic construct
  misc_feature 1..28: note = primer
  source 1..28: mol_type = other DNA
gttcagagactgaaggatgaagccagag

SEQ ID NO: 654 - DNA, 29 bp, synthetic construct
  misc_feature 1..29: note = primer
  source 1..29: mol_type = other DNA
gatgatgatgaagaggatgatgatgaaga

SEQ ID NO: 655 - DNA, 24 bp, synthetic construct
  misc_feature 1..24: note = primer
  source 1..24: mol_type = other DNA
cagcttccagtcacagcataggct

SEQ ID NO: 656 - DNA, 28 bp, synthetic construct
  misc_feature 1..28: note = primer
  source 1..28: mol_type = other DNA
gatgtcagcatacccagatccacattag

SEQ ID NO: 657 - DNA, 24 bp, synthetic construct
  misc_feature 1..24: note = primer
  source 1..24: mol_type = other DNA
accgcttggaatgctgcaacaatg

SEQ ID NO: 658 - DNA, 27 bp, synthetic construct
  misc_feature 1..27: note = primer
  source 1..27: mol_type = other DNA
ccaacaggcatgataggatatggaatt

SEQ ID NO: 659 - DNA, 25 bp, synthetic construct
  misc_feature 1..25: note = primer
  source 1..25: mol_type = other DNA
ctttggccctgtatcaggagcacag

SEQ ID NO: 660 - DNA, 21 bp, synthetic construct
  misc_feature 1..21: note = primer
  source 1..21: mol_type = other DNA
ctgggccagaatggtgaggag

SEQ ID NO: 661 - DNA, 23 bp, synthetic construct
  misc_feature 1..23: note = primer
  source 1..23: mol_type = other DNA
ggctggcgcacctgggcgtgcag

SEQ ID NO: 662 - DNA, 25 bp, synthetic construct
  misc_feature 1..25: note = primer
  source 1..25: mol_type = other DNA
tgatgaaggggaggaaggagaggaa

SEQ ID NO: 663 - DNA, 28 bp, synthetic construct
  misc_feature 1..28: note = primer
  source 1..28: mol_type = other DNA
cgtgatctttgatagatccagggaagag

SEQ ID NO: 664 - DNA, 27 bp, synthetic construct
  misc_feature 1..27: note = primer
  source 1..27: mol_type = other DNA
ttcttggctcaacaagccataaaacag

SEQ ID NO: 665 - DNA, 29 bp, synthetic construct
  misc_feature 1..29: note = primer
  source 1..29: mol_type = other DNA
ggtcttataatcttccctctcttccggat

SEQ ID NO: 666 - DNA, 25 bp, synthetic construct
  misc_feature 1..25: note = primer
  source 1..25: mol_type = other DNA
ccagcaaccttccatctctcatcag

SEQ ID NO: 667 - DNA, 24 bp, synthetic construct
  misc_feature 1..24: note = primer
  source 1..24: mol_type = other DNA
ccgcagagcactgtatcacgaaaa

SEQ ID NO: 668 - DNA, 30 bp, synthetic construct
  misc_feature 1..30: note = primer
  source 1..30: mol_type = other DNA
attcctgttcctactacagttcctgttcct

SEQ ID NO: 669 - DNA, 29 bp, synthetic construct
  misc_feature 1..29: note = primer
  source 1..29: mol_type = other DNA
accaagaggctattcaagatctctgcctg

SEQ ID NO: 670 - DNA, 29 bp, synthetic construct
  misc_feature 1..29: note = primer
  source 1..29: mol_type = other DNA
accaagaggctattcaagatctctgtatg

SEQ ID NO: 671 - DNA, 30 bp, synthetic construct
  misc_feature 1..30: note = primer
  source 1..30: mol_type = other DNA
agcatttggttttaaattatggagtatgtt

SEQ ID NO: 672 - DNA, 26 bp, synthetic construct
  misc_feature 1..26: note = primer
  source 1..26: mol_type = other DNA
tctgtggagacgagaatattctggtt

SEQ ID NO: 673 - DNA, 27 bp, synthetic construct
  misc_feature 1..27: note = primer
  source 1..27: mol_type = other DNA
gatttgtgattttggtctagccagagt

SEQ ID NO: 674 - DNA, 31 bp, synthetic construct
  misc_feature 1..31: note = primer
  source 1..31: mol_type = other DNA
catcaagaatgattctaattatgtggttaaa

SEQ ID NO: 675 - DNA, 42 bp, synthetic construct
  misc_feature 1..42: note = primer
  source 1..42: mol_type = other DNA
ctctacgtctcctccgagagccgtccaacccttagggaaccc

SEQ ID NO: 676 - DNA, 42 bp, synthetic construct; identical to SEQ ID NO: 298
  misc_feature 1..42: note = primer
  source 1..42: mol_type = other DNA

SEQ ID NO: 677 - DNA, 48 bp, synthetic construct; identical to SEQ ID NO: 256
  misc_feature 1..48: note = primer
  source 1..48: mol_type = other DNA

SEQ ID NO: 678 - DNA, 47 bp, synthetic construct
  misc_feature 1..47: note = primer
  source 1..47: mol_type = other DNA
aaatgacatcagatgtaccatcactgggtccaacccttagggaaccc

SEQ ID NO: 679 - DNA, 48 bp, synthetic construct
  misc_feature 1..48: note = primer
  source 1..48: mol_type = other DNA
tcgcttcatggagatataagtagcctgaatccaacccttagggaaccc

SEQ ID NO: 680 - DNA, 46 bp, synthetic construct
  misc_feature 1..46: note = primer
  source 1..46: mol_type = other DNA
gaaattcggcgccttcatcagtatgtgtccaacccttagggaaccc

SEQ ID NO: 681 - DNA, 49 bp, synthetic construct
  misc_feature 1..49: note = primer
  source 1..49: mol_type = other DNA
atcgtactgagaagcactccacaatgccagtccaacccttagggaaccc

SEQ ID NO: 682 - DNA, 52 bp, synthetic construct
  misc_feature 1..52: note = primer
  source 1..52: mol_type = other DNA
caggaacgaatttcatatacacctccagagagctccaacccttagggaaccc

SEQ ID NO: 683 - DNA, 45 bp, synthetic construct
  misc_feature 1..45: note = primer
  source 1..45: mol_type = other DNA
gcttgcagccaatttactggagcaggtccaacccttagggaaccc

SEQ ID NO: 684 - DNA, 52 bp, synthetic construct
  misc_feature 1..52: note = primer
  source 1..52: mol_type = other DNA
atcttagacgaattttacaatgtgaagttctgctccaacccttagggaaccc

SEQ ID NO: 685 - DNA, 43 bp, synthetic construct
  misc_feature 1..43: note = primer
  source 1..43: mol_type = other DNA
gtgtctgctgactccagtgcatcctccaacccttagggaaccc

SEQ ID NO: 686 - DNA, 47 bp, synthetic construct
  misc_feature 1..47: note = primer
  source 1..47: mol_type = other DNA
attgtcagtaaacgggcaggtactcagttccaacccttagggaaccc

SEQ ID NO: 687 - DNA, 46 bp, synthetic construct
  misc_feature 1..46: note = primer
  source 1..46: mol_type = other DNA
gatgacagcatggaagagaaaccactatccaacccttagggaaccc

SEQ ID NO: 688 - DNA, 42 bp, synthetic construct
  misc_feature 1..42: note = primer
  source 1..42: mol_type = other DNA
tgagtccattcttgcacaccgagtccaacccttagggaaccc

SEQ ID NO: 689 - DNA, 46 bp, synthetic construct
  misc_feature 1..46: note = primer
  source 1..46: mol_type = other DNA
attttggctctctatttgacttggagctccaacccttagggaaccc

SEQ ID NO: 690 - DNA, 43 bp, synthetic construct
  misc_feature 1..43: note = primer
  source 1..43: mol_type = other DNA
atgggaatgatgaacaaccccaattccaacccttagggaaccc

SEQ ID NO: 691 - DNA, 42 bp, synthetic construct
  misc_feature 1..42: note = primer
  source 1..42: mol_type = other DNA
ccttgccctttaaggtggtggtgtccaacccttagggaaccc

SEQ ID NO: 692 - DNA, 47 bp, synthetic construct
  misc_feature 1..47: note = primer
  source 1..47: mol_type = other DNA
ctgccttatgactcaagatgggagtttctccaacccttagggaaccc

SEQ ID NO: 693 - DNA, 42 bp, synthetic construct
  misc_feature 1..42: note = primer
  source 1..42: mol_type = other DNA
gtgccagcaagatccaatctagagggccccgagaacctcgaa

SEQ ID NO: 694 - DNA, 45 bp, synthetic construct
  misc_feature 1..45: note = primer
  source 1..45: mol_type = other DNA
gtgccagcaagatccaatctagagggcgacctcttccagaagctg

SEQ ID NO: 695 - DNA, 50 bp, synthetic construct
  misc_feature 1..50: note = primer
  source 1..50: mol_type = other DNA
gtgccagcaagatccaatctagaatccaacgaccaagaactctctggaaa

SEQ ID NO: 696 - DNA, 48 bp, synthetic construct
  misc_feature 1..48: note = primer
  source 1..48: mol_type = other DNA
gtgccagcaagatccaatctagagccagttatttgcctcagtaaaaga

SEQ ID NO: 697 - DNA, 53 bp, synthetic construct
  misc_feature 1..53: note = primer
  source 1..53: mol_type = other DNA
gtgccagcaagatccaatctagacagtatgagtacacggagctcaagaaacag

SEQ ID NO: 698 - DNA, 48 bp, synthetic construct
  misc_feature 1..48: note = primer
  source 1..48: mol_type = other DNA
gtgccagcaagatccaatctagacacaatgaaacagatttgcaaaaag

SEQ ID NO: 699 - DNA, 43 bp, synthetic construct
  misc_feature 1..43: note = primer
  source 1..43: mol_type = other DNA
gtgccagcaagatccaatctagaccgcctgcctgcgcacctgc

SEQ ID NO: 700 - DNA, 45 bp, synthetic construct
  misc_feature 1..45: note = primer
  source 1..45: mol_type = other DNA
gtgccagcaagatccaatctagacgggcagaaggctggtgacaag

SEQ ID NO: 701 - DNA, 43 bp, synthetic construct
  misc_feature 1..43: note = primer
  source 1..43: mol_type = other DNA
gtgccagcaagatccaatctagaaagggccaaccactggggaa

SEQ ID NO: 702 - DNA, 53 bp, synthetic construct
  misc_feature 1..53: note = primer
  source 1..53: mol_type = other DNA
gtgccagcaagatccaatctagaaaaacaagtgcacagacaacaccaagtaag

SEQ ID NO: 703 - DNA, 49 bp, synthetic construct
  misc_feature 1..49: note = primer
  source 1..49: mol_type = other DNA
gtgccagcaagatccaatctagagggcaaatgagaacagcaacatacag

SEQ ID NO: 704 - DNA, 49 bp, synthetic construct
  misc_feature 1..49: note = primer
  source 1..49: mol_type = other DNA
gtgccagcaagatccaatctagagcacacctctcaatgcagctttacag

SEQ ID NO: 705 - DNA, 52 bp, synthetic construct
  misc_feature 1..52: note = primer
  source 1..52: mol_type = other DNA
gtgccagcaagatccaatctagatgaggacaagttctacagccacaagaaaa

SEQ ID NO: 706 - DNA, 44 bp, synthetic construct
  misc_feature 1..44: note = primer
  source 1..44: mol_type = other DNA
gtgccagcaagatccaatctagaagaaccccaacagcaaagaag

SEQ ID NO: 707 - DNA, 45 bp, synthetic construct
  misc_feature 1..45: note = primer
  source 1..45: mol_type = other DNA
gtgccagcaagatccaatctagacctaagatgcccgacttcaact

SEQ ID NO: 708 - DNA, 47 bp, synthetic construct
  misc_feature 1..47: note = primer
  source 1..47: mol_type = other DNA
gtgccagcaagatccaatctagaaatgctggacttccgtagtgacca

SEQ ID NO: 709 - DNA, 53 bp, synthetic construct
  misc_feature 1..53: note = primer
  source 1..53: mol_type = other DNA
gtgccagcaagatccaatctagacagtgccaggaaagagaattagagatcagt

SEQ ID NO: 710 - DNA, 46 bp, synthetic construct
  misc_feature 1..46: note = primer
  source 1..46: mol_type = other DNA
gtgccagcaagatccaatctagacaaagcccacgctgaagcgaaag

SEQ ID NO: 711 - DNA, 49 bp, synthetic construct
  misc_feature 1..49: note = primer
  source 1..49: mol_type = other DNA
gtgccagcaagatccaatctagaagctaaaaggacagcaggtgctacca

SEQ ID NO: 712 - DNA, 51 bp, synthetic construct
  misc_feature 1..51: note = primer
  source 1..51: mol_type = other DNA
gtgccagcaagatccaatctagagttcagagactgaaggatgaagccagag

SEQ ID NO: 713 - DNA, 52 bp, synthetic construct
  misc_feature 1..52: note = primer
  source 1..52: mol_type = other DNA
gtgccagcaagatccaatctagagatgatgatgaagaggatgatgatgaaga

SEQ ID NO: 714 - DNA, 44 bp, synthetic construct
  misc_feature 1..44: note = primer
  source 1..44: mol_type = other DNA
gtgccagcaagatccaatctagacagcttccagtcacagcatag

SEQ ID NO: 715 - DNA, 51 bp, synthetic construct
  misc_feature 1..51: note = primer
  source 1..51: mol_type = other DNA
gtgccagcaagatccaatctagagatgtcagcatacccagatccacattag

SEQ ID NO: 716 - DNA, 47 bp, synthetic construct
  misc_feature 1..47: note = primer
  source 1..47: mol_type = other DNA
gtgccagcaagatccaatctagaaccgcttggaatgctgcaacaatg

SEQ ID NO: 717 - DNA, 50 bp, synthetic construct
  misc_feature 1..50: note = primer
  source 1..50: mol_type = other DNA
gtgccagcaagatccaatctagaccaacaggcatgataggatatggaatt

SEQ ID NO: 718 - DNA, 48 bp, synthetic construct
  misc_feature 1..48: note = primer
  source 1..48: mol_type = other DNA
gtgccagcaagatccaatctagactttggccctgtatcaggagcacag

SEQ ID NO: 719 - DNA, 44 bp, synthetic construct
  misc_feature 1..44: note = primer
  source 1..44: mol_type = other DNA
gtgccagcaagatccaatctagactgggccagaatggtgaggag

SEQ ID NO: 720 - DNA, 46 bp, synthetic construct
  misc_feature 1..46: note = primer
  source 1..46: mol_type = other DNA
gtgccagcaagatccaatctagaggctggcgcacctgggcgtgcag

SEQ ID NO: 721 - DNA, 45 bp, synthetic construct
  misc_feature 1..45: note = primer
  source 1..45: mol_type = other DNA
gtgccagcaagatccaatctagatgatgaaggggaggaaggagag

SEQ ID NO: 722 - DNA, 51 bp, synthetic construct
  misc_feature 1..51: note = primer
  source 1..51: mol_type = other DNA
gtgccagcaagatccaatctagacgtgatctttgatagatccagggaagag

SEQ ID NO: 723 - DNA, 50 bp, synthetic construct
  misc_feature 1..50: note = primer
  source 1..50: mol_type = other DNA
gtgccagcaagatccaatctagattcttggctcaacaagccataaaacag

SEQ ID NO: 724 - DNA, 52 bp, synthetic construct
  misc_feature 1..52: note = primer
  source 1..52: mol_type = other DNA
gtgccagcaagatccaatctagaggtcttataatcttccctctcttccggat

SEQ ID NO: 725 - DNA, 48 bp, synthetic construct
  misc_feature 1..48: note = primer
  source 1..48: mol_type = other DNA
gtgccagcaagatccaatctagaccagcaaccttccatctctcatcag

SEQ ID NO: 726 - DNA, 47 bp, synthetic construct
  misc_feature 1..47: note = primer
  source 1..47: mol_type = other DNA
gtgccagcaagatccaatctagaccgcagagcactgtatcacgaaaa

SEQ ID NO: 727 - DNA, 53 bp, synthetic construct
  misc_feature 1..53: note = primer
  source 1..53: mol_type = other DNA
gtgccagcaagatccaatctagaattcctgttcctactacagttcctgttcct

SEQ ID NO: 728 - DNA, 52 bp, synthetic construct
  misc_feature 1..52: note = primer
  source 1..52: mol_type = other DNA
gtgccagcaagatccaatctagaaccaagaggctattcaagatctctgcctg

SEQ ID NO: 729 - DNA, 52 bp, synthetic construct
  misc_feature 1..52: note = primer
  source 1..52: mol_type = other DNA
gtgccagcaagatccaatctagaaccaagaggctattcaagatctctgtatg

SEQ ID NO: 730 - DNA, 53 bp, synthetic construct
  misc_feature 1..53: note = primer
  source 1..53: mol_type = other DNA
gtgccagcaagatccaatctagaagcatttggttttaaattatggagtatgtt

SEQ ID NO: 731 - DNA, 45 bp, synthetic construct
  misc_feature 1..45: note = primer
  source 1..45: mol_type = other DNA
tctgtggagacgagaatattctggtttccaacccttagggaaccc

SEQ ID NO: 732 - DNA, 50 bp, synthetic construct
  misc_feature 1..50: note = primer
  source 1..50: mol_type = other DNA
gtgccagcaagatccaatctagagatttgtgattttggtctagccagagt

SEQ ID NO: 733 - DNA, 50 bp, synthetic construct
  misc_feature 1..50: note = primer
  source 1..50: mol_type = other DNA
catcaagaatgattctaattatgtggttaaatccaacccttagggaaccc

SEQ ID NO: 734 - DNA, 30 bp, synthetic construct
  misc_feature 1..30: note = primer
  source 1..30: mol_type = other DNA
ggagcggacatggactacgactcgtaccag

SEQ ID NO: 735 - DNA, 30 bp, synthetic construct
  misc_feature 1..30: note = primer
  source 1..30: mol_type = other DNA
agaatgaagaaattgatgttgtgacagtag

SEQ ID NO: 736 - DNA, 26 bp, synthetic construct
  misc_feature 1..26: note = primer
  source 1..26: mol_type = other DNA
aggaaggcatcaaccacgagtgtaag

SEQ ID NO: 737 - DNA, 28 bp, synthetic construct
  misc_feature 1..28: note = primer
  source 1..28: mol_type = other DNA
cagctctctgtaatgcgatactcaccag

SEQ ID NO: 738 - DNA, 30 bp, synthetic construct
  misc_feature 1..30: note = primer
  source 1..30: mol_type = other DNA
aagagaggctgtatctccatgccagagcag

SEQ ID NO: 739 - DNA, 30 bp, synthetic construct
  misc_feature 1..30: note = primer
  source 1..30: mol_type = other DNA
acccaaaagcagaccttggagaacagtcag

SEQ ID NO: 740 - DNA, 30 bp, synthetic construct
  misc_feature 1..30: note = primer
  source 1..30: mol_type = other DNA
tctttgaactactgccggagcttctgccag

SEQ ID NO: 741 - DNA, 24 bp, synthetic construct
  misc_feature 1..24: note = primer
  source 1..24: mol_type = other DNA
caccgaggaccagctcaatgacag

SEQ ID NO: 742 - DNA, 49 bp, synthetic construct
  misc_feature 1..49: note = primer
  source 1..49: mol_type = other DNA
ggagcggacatggactacgactcgtaccagtccaacccttagggaaccc

SEQ ID NO: 743 - DNA, 49 bp, synthetic construct
  misc_feature 1..49: note = primer
  source 1..49: mol_type = other DNA
agaatgaagaaattgatgttgtgacagtagtccaacccttagggaaccc

SEQ ID NO: 744 - DNA, 45 bp, synthetic construct
  misc_feature 1..45: note = primer
  source 1..45: mol_type = other DNA
aggaaggcatcaaccacgagtgtaagtccaacccttagggaaccc

SEQ ID NO: 745 - DNA, 51 bp, synthetic construct
  misc_feature 1..51: note = primer
  source 1..51: mol_type = other DNA
gtgccagcaagatccaatctagacagctctctgtaatgcgatactcaccag

SEQ ID NO: 746 - DNA, 53 bp, synthetic construct
  misc_feature 1..53: note = primer
  source 1..53: mol_type = other DNA
gtgccagcaagatccaatctagaaagagaggctgtatctccatgccagagcag

SEQ ID NO: 747 - DNA, 53 bp, synthetic construct
  misc_feature 1..53: note = primer
  source 1..53: mol_type = other DNA
gtgccagcaagatccaatctagaacccaaaagcagaccttggagaacagtcag

SEQ ID NO: 748 - DNA, 53 bp, synthetic construct
  misc_feature 1..53: note = primer
  source 1..53: mol_type = other DNA
gtgccagcaagatccaatctagatctttgaactactgccggagcttctgccag

SEQ ID NO: 749 - DNA, 47 bp, synthetic construct
  misc_feature 1..47: note = primer
  source 1..47: mol_type = other DNA
gtgccagcaagatccaatctagacaccgaggaccagctcaatgacag

SEQ ID NO: 750 - DNA, 22 bp, synthetic construct
  misc_feature 1..22: note = primer
  source 1..22: mol_type = other DNA
catcccagtgactgcatccctc

SEQ ID NO: 751 - DNA, 20 bp, synthetic construct
  misc_feature 1..20: note = primer
  source 1..20: mol_type = other DNA
ggggaccccattcccgagga

SEQ ID NO: 752 - DNA, 27 bp, synthetic construct
  misc_feature 1..27: note = primer
  source 1..27: mol_type = other DNA
gctctccacagatagagaacatccagc

SEQ ID NO: 753 - DNA, 25 bp, synthetic construct
  misc_feature 1..25: note = primer
  source 1..25: mol_type = other DNA
ctgaacagatgggtaaggatggcag

SEQ ID NO: 754 - DNA, 23 bp, synthetic construct
  misc_feature 1..23: note = primer
  source 1..23: mol_type = other DNA
ggaccaaccacttcctaccccag

SEQ ID NO: 755 - DNA, 20 bp, synthetic construct
  misc_feature 1..20: note = primer
  source 1..20: mol_type = other DNA
gccccaggtgtacccaccac

SEQ ID NO: 756 - DNA, 20 bp, synthetic construct
  misc_feature 1..20: note = primer
  source 1..20: mol_type = other DNA
gcctcacctgcagatgcccc

SEQ ID NO: 757 - DNA, 25 bp, synthetic construct
  misc_feature 1..25: note = primer
  source 1..25: mol_type = other DNA
gcaacctccaagtcccagatcatgt

SEQ ID NO: 758 - DNA, 20 bp, synthetic construct
  misc_feature 1..20: note = primer
  source 1..20: mol_type = other DNA
ggagttcctggtcggctccg

SEQ ID NO: 759 - DNA, 27 bp, synthetic construct
  misc_feature 1..27: note = primer
  source 1..27: mol_type = other DNA
cgagttcaagcaggcctatatcacctg

SEQ ID NO: 760 - DNA, 20 bp, synthetic construct
  misc_feature 1..20: note = primer
  source 1..20: mol_type = other DNA
gtgggcctcctgggcctcag

SEQ ID NO: 761 - DNA, 23 bp, synthetic construct
  misc_feature 1..23: note = primer
  source 1..23: mol_type = other DNA
tccctggaatgaagggacacaga

SEQ ID NO: 762 - DNA, 20 bp, synthetic construct
  misc_feature 1..20: note = primer
  source 1..20: mol_type = other DNA
atggcaaaactggcccccct

SEQ ID NO: 763 - DNA, 23 bp, synthetic construct
  misc_feature 1..23: note = primer
  source 1..23: mol_type = other DNA
tccctggacctaaaggtgctgct

SEQ ID NO: 764 - DNA, 23 bp, synthetic construct
  misc_feature 1..23: note = primer
  source 1..23: mol_type = other DNA
aagcaggcaaacctggtgaacag

SEQ ID NO: 765 - DNA, 22 bp, synthetic construct
  misc_feature 1..22: note = primer
  source 1..22: mol_type = other DNA
tccagggcctaagggtgacaga

SEQ ID NO: 766 - DNA, 20 bp, synthetic construct
  misc_feature 1..20: note = primer
  source 1..20: mol_type = other DNA
ctggtgcccctggtgacaag

SEQ ID NO: 767 - DNA, 20 bp, synthetic construct
  misc_feature 1..20: note = primer
  source 1..20: mol_type = other DNA
ctggaccccctggccccatt

SEQ ID NO: 768 - DNA, 20 bp, synthetic construct
  misc_feature 1..20: note = primer
  source 1..20: mol_type = other DNA
agggtccccctggccctcct

SEQ ID NO: 769 - DNA, 20 bp, synthetic construct
  misc_feature 1..20: note = primer
  source 1..20: mol_type = other DNA
ctggtcctgctggtccccga

SEQ ID NO: 770 - DNA, 20 bp, synthetic construct
  misc_feature 1..20: note = primer
  source 1..20: mol_type = other DNA
ctggcgagcctggagcttca

SEQ ID NO: 771 - DNA, 22 bp, synthetic construct
  misc_feature 1..22: note = primer
  source 1..22: mol_type = other DNA
tgtcctccttgaagggctccag

SEQ ID NO: 772 - DNA, 27 bp, synthetic construct
  misc_feature 1..27: note = primer
  source 1..27: mol_type = other DNA
cctccactgaagaagctgaaacaagag

SEQ ID NO: 773 - DNA, 25 bp, synthetic construct
  misc_feature 1..25: note = primer
  source 1..25: mol_type = other DNA
gagagtctggatggacatttgcagg

SEQ ID NO: 774 - DNA, 20 bp, synthetic construct
  misc_feature 1..20: note = primer
  source 1..20: mol_type = other DNA
tgcgaagccacctctcgcag

SEQ ID NO: 775 - DNA, 41 bp, synthetic construct
  misc_feature 1..41: note = primer
  source 1..41: mol_type = other DNA
catcccagtgactgcatccctctccaacccttagggaaccc

SEQ ID NO: 776 - DNA, 39 bp, synthetic construct
  misc_feature 1..39: note = primer
  source 1..39: mol_type = other DNA
ggggaccccattcccgaggatccaacccttagggaaccc

SEQ ID NO: 777 - DNA, 46 bp, synthetic construct
  misc_feature 1..46: note = primer
  source 1..46: mol_type = other DNA
gctctccacagatagagaacatccagctccaacccttagggaaccc

SEQ ID NO: 778 - DNA, 44 bp, synthetic construct
  misc_feature 1..44: note = primer
  source 1..44: mol_type = other DNA
ctgaacagatgggtaaggatggcagtccaacccttagggaaccc

SEQ ID NO: 779 - DNA, 42 bp, synthetic construct
  misc_feature 1..42: note = primer
  source 1..42: mol_type = other DNA
ggaccaaccacttcctaccccagtccaacccttagggaaccc

SEQ ID NO: 780 - DNA, 39 bp, synthetic construct
  misc_feature 1..39: note = primer
  source 1..39: mol_type = other DNA
gccccaggtgtacccaccactccaacccttagggaaccc

SEQ ID NO: 781 - DNA, 39 bp, synthetic construct
  misc_feature 1..39: note = primer
  source 1..39: mol_type = other DNA
gcctcacctgcagatgcccctccaacccttagggaaccc

SEQ ID NO: 782 - DNA, 44 bp, synthetic construct
  misc_feature 1..44: note = primer
  source 1..44: mol_type = other DNA
gcaacctccaagtcccagatcatgttccaacccttagggaaccc

SEQ ID NO: 783 - DNA, 39 bp, synthetic construct
  misc_feature 1..39: note = primer
  source 1..39: mol_type = other DNA
ggagttcctggtcggctccgtccaacccttagggaaccc

SEQ ID NO: 784 - DNA, 50 bp, synthetic construct
  misc_feature 1..50: note = primer
  source 1..50: mol_type = other DNA
gtgccagcaagatccaatctagacgagttcaagcaggcctatatcacctg

SEQ ID NO: 785 - DNA, 43 bp, synthetic construct
  misc_feature 1..43: note = primer
  source 1..43: mol_type = other DNA
gtgccagcaagatccaatctagagtgggcctcctgggcctcag

SEQ ID NO: 786 - DNA, 46 bp, synthetic construct
  misc_feature 1..46: note = primer
  source 1..46: mol_type = other DNA
gtgccagcaagatccaatctagatccctggaatgaagggacacaga

SEQ ID NO: 787 - DNA, 43 bp, synthetic construct
  misc_feature 1..43: note = primer
  source 1..43: mol_type = other DNA
gtgccagcaagatccaatctagaatggcaaaactggcccccct

SEQ ID NO: 788 - DNA, 46 bp, synthetic construct
  misc_feature 1..46: note = primer
  source 1..46: mol_type = other DNA
gtgccagcaagatccaatctagatccctggacctaaaggtgctgct

SEQ ID NO: 789 - DNA, 46 bp, synthetic construct
  misc_feature 1..46: note = primer
  source 1..46: mol_type = other DNA
gtgccagcaagatccaatctagaaagcaggcaaacctggtgaacag

SEQ ID NO: 790 - DNA, 45 bp, synthetic construct
  misc_feature 1..45: note = primer
  source 1..45: mol_type = other DNA
gtgccagcaagatccaatctagatccagggcctaagggtgacaga

SEQ ID NO: 791 - DNA, 43 bp, synthetic construct
  misc_feature 1..43: note = primer
  source 1..43: mol_type = other DNA
gtgccagcaagatccaatctagactggtgcccctggtgacaag

SEQ ID NO: 792 - DNA, 43 bp, synthetic construct
  misc_feature 1..43: note = primer
  source 1..43: mol_type = other DNA
gtgccagcaagatccaatctagactggaccccctggccccatt

SEQ ID NO: 793 - DNA, 43 bp, synthetic construct
  misc_feature 1..43: note = primer
  source 1..43: mol_type = other DNA
gtgccagcaagatccaatctagaagggtccccctggccctcct

SEQ ID NO: 794 - DNA, 43 bp, synthetic construct
  misc_feature 1..43: note = primer
  source 1..43: mol_type = other DNA
gtgccagcaagatccaatctagactggtcctgctggtccccga

SEQ ID NO: 795 - DNA, 43 bp, synthetic construct
  misc_feature 1..43: note = primer
  source 1..43: mol_type = other DNA
gtgccagcaagatccaatctagactggcgagcctggagcttca

SEQ ID NO: 796 - DNA, 45 bp, synthetic construct
  misc_feature 1..45: note = primer
  source 1..45: mol_type = other DNA
gtgccagcaagatccaatctagatgtcctccttgaagggctccag

SEQ ID NO: 797 - DNA, 50 bp, synthetic construct
  misc_feature 1..50: note = primer
  source 1..50: mol_type = other DNA
gtgccagcaagatccaatctagacctccactgaagaagctgaaacaagag

SEQ ID NO: 798 - DNA, 48 bp, synthetic construct
  misc_feature 1..48: note = primer
  source 1..48: mol_type = other DNA
gtgccagcaagatccaatctagagagagtctggatggacatttgcagg

SEQ ID NO: 799 - DNA, 43 bp, synthetic construct
  misc_feature 1..43: note = primer
  source 1..43: mol_type = other DNA
gtgccagcaagatccaatctagatgcgaagccacctctcgcag